(19) United States
(12) Patent Application Publication
(10) Pub. No.: US 2025/0092261 A1
(43) Pub. Date: Mar. 20, 2025

(54) Dibenzoxanthene Quenchers, Uses, and Methods of Preparation

(71) Applicant: Life Technologies Corporation, Carlsbad, CA (US)

(72) Inventors: Andrew Levitz, San Francisco, CA (US); Scott Benson, Alameda, CA (US); Brian Evans, Mountain View, CA (US); Khairuzzaman Bashar Mullah, Union City, CA (US)

(21) Appl. No.: 18/580,853

(22) PCT Filed: Jul. 21, 2022
(86) PCT No.: PCT/US2022/074028

§ 371 Date: Jan. 19, 2024

Related U.S. Application Data
(60) Provisional application No. 63/224,300, filed on Jul. 21, 2021.

(51) Int. Cl. C09B 57/14; C12Q 1/686; G01N 21/64

(52) U.S. Cl. CPC C09B 57/14; C12Q 1/686; G01N 21/6428; G01N 2021/6432

(57) ABSTRACT

The present disclosure relates to dibenzoxanthene compounds that are efficient quenchers of fluorescence, for example in the far red and near infrared spectrum. Applications using the dibenzoxanthene quenching compounds and methods of making same are also described. Also disclosed is a method of detecting or quantifying a target nucleic acid molecule in a sample by polymerase chain reaction (PCR). The compounds have the following formula (I).

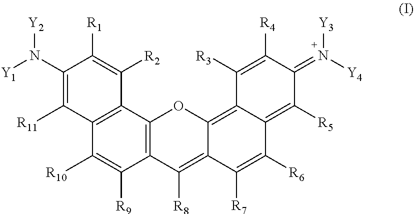

### FIELD

Disclosed herein are dibenzoxanthene compounds that are efficient quenchers of fluorescence, for example in the far red and near infrared spectrum. Applications using the dibenzoxanthene quenching compounds and methods of making same are also described.

### BACKGROUND

The use of a variety of dyes to quench fluorescence is known in the art. The application of this phenomenon to analyze biological systems is also well-detailed. Chemical moieties that quench fluorescent light operate through a variety of mechanisms, including fluorescent energy transfer (FRET) processes and ground state quenching. The energy transfer process frequently requires overlap between the emission spectrum of the fluorescent donor and the absorbance spectrum of the quencher. The need for spectral overlap can complicate the design of dye-quencher pairs, because not all potential combinations of quenchers and donors can be used.

Quenching compounds have been used in a variety of energy-transfer assays and applications, especially for use in dye-quencher pairs for use in genetic and protein analysis assays. For example, quenchers have been used with reporter dyes that generate fluorescence in the visible region of electromagnetic spectrum, for example, in WO 2000/064988 and WO 2002/012195. For a quencher to be effective, such compounds must be able to efficiently absorb energy from the dye, such that energy transfer from the donor to the quencher results in little or no residual fluorescence emission by the quencher. For this reason, it may be preferable to use a quencher compound that exhibits minimal or no detectable fluorescence if excited at the wavelengths being used to excite the donor dye. Further, for a quencher to be useful in certain biological applications, such as qPCR assays, the quencher must remain stable under the demanding and harsh conditions of the assay. In addition, the use of varied quenchers complicates assay development because the purification of a given probe can vary greatly depending on the nature of the quencher attached. Thus, a desirable quencher must be stable enough to absorb energy from the dye and withstand harsh chemical conditions and the rigors of automated DNA synthesis.

Unfortunately, quenchers that effectively quench fluorescence of dyes that emit in the far red and near-infrared region are far less common. Efficient quenching of fluorescent dyes that operate in the far-red and near-IR spectral regions is problematic for a variety of reasons. For example, many known quenchers do not absorb energy from fluorophores that emit in the far-red or near-IR spectral region, while other types of materials that can function as quenchers in this spectral region, such as gold nanoparticle quenchers, are too large. Furthermore, it has been antidotally found that the farther the excitation/emission wavelength of compounds shift to the red region of the spectrum, the higher incidence of stability issues. Thus, there is a need in the art for quenchers that are both thermally and photolytically stable, and are able to quench fluorescence of compounds that emit over a range of wavelengths.

The compounds of the present disclosure are new and highly useful quenchers, in particular, in quenching fluorescence from compounds that absorb and/or emit light in the far red and near infrared regions of the electromagnetic spectrum.

### BRIEF SUMMARY

Accordingly, the present disclosure relates to quenchers chosen from compounds of Formula (I):

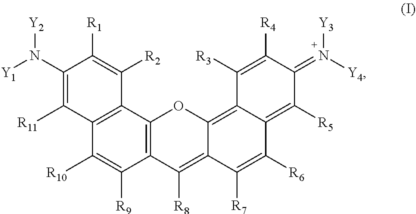

wherein:
Y₁ is selected from Y₁′ and —C(O)R″,
Y₂ is selected from Y₂′ and —C(O)R″ on the condition that Y₁ and Y₂ are not both —C(O)R″;
or, alternatively, Y₁ and Y₂ form N═NR′ with the nitrogen to which they are bound;
or, alternatively, Y₁′ forms a saturated or unsaturated, substituted or unsubstituted ring with R₁/R₁₁ together with the atoms to which they are bonded, and/or Y₂′ forms a saturated or unsaturated, substituted or unsubstituted ring with R₁/R₁₁ together with the atoms to which they are bonded;
Y₃ is selected from Y₃′ and —C(O)R″,
Y₄ is selected from Y₄′ and —C(O)R″ on the condition that Y₃ and Y₄ are not both —C(O)R″;
or, alternatively, Y₃′ forms a saturated or unsaturated, substituted or unsubstituted ring with R₄/R₅ together with the atoms to which they are bonded, and/or Y₄′ forms a saturated or unsaturated, substituted or unsubstituted ring with R₄/R₅ together with the atoms to which they are bonded;
R″ is selected from —(CQ¹Q²)ₓ-R^{a};
wherein Q¹ and Q² are independently selected from hydrogen and methyl,
x is an integer ranging from 1 to 10,
R^{a} is a trimethyl quinone;
R₅, R₆, R₇, R₉, R₁₀, R₁₁ are independently selected from —H, halogens, alkyl, and alkyl group independently substituted with one or more Z₂;
R₁, R₂, R₃, R₄, Y₁′, Y₂′, Y₃′, Y₄′, and R′ are independently selected from —H, alkyl, alkyl independently substituted with one or more Z₂, heteroalkyl, heteroalkyl independently substituted with one or more Z₂, aryl, aryl independently substituted with one or more Z₂, heteroaryl, heteroaryl independently substituted with one or more Z₂, arylalkyl, arylalkyl independently substituted with one or more Z₂, heteroarylalkyl, heteroarylalkyl independently substituted with one or more Z₂, halogen, —OS(O)₂OR, —S(O)₂OR, —S(O)₂R, —S(O)₂NR, —S(O)R, —OP(O)O₂RR, —P(O)O₂RR, —C(O)OR, —NO₂, ═NRR, —NRR, —N⁺RRR, —NC(O)R, —C(O)R, —C(O)NRR, —CN, and —OR;
wherein R is independently selected from —H, alkyl, heteroalkyl, aryl, heteroaryl, arylalkyl, and heteroarylalkyl;
wherein Z₂ is selected from —R, halogen, —OS(O)₂OR, —S(O)₂OR, —S(O)₂R, —S(O)₂NR, —S(O)R, —OP(O)O₂RR—P(O)O₂RR, —C(O)OR, —NO₂, —NRR, —N⁺RRR, —NC(O)R, —C(O)R, —C(O)NRR, —CN, —O, —OR, —(CH₂)ₓ—R^{b}, —N(CH₂)ₓ—R^{b};
wherein R^{b} is selected from -halogen, —OH, —OR, —SH, —NH₂, —C(O)O, —C(O)OH, —C(O)NH₂;
R₈ is selected from —H, alkyl, alkyl independently substituted with one or more Z₁, heteroalkyl, heteroalkyl independently substituted with one or more Z₁, aryl, aryl independently substituted with one or more Z₁, heteroaryl, heteroaryl independently substituted with one or more Z₁, arylalkyl, arylalkyl independently substituted with one or more Z₁, heteroarylalkyl, and heteroarylalkyl independently substituted with one or more Z₁; and
Z₁ is selected from the group consisting of, —R*, halogen, —CR*R*R*, —OS(O)₂OR*, —S(O)₂OR*, —SO₃, —S(O)₂R*, —S(O)₂NR*, —S(O)R*, —OP(O)O₂R*R*—P(O)O₂R*R*, —C(O)OR*, —N═N—R*—R*, —NO₂—NR*R*, —N*R*R*R*, —NC(O)R*, —C(O)R*, —C(O)NR*R*, —CN, —O and —OR*, wherein R* is independently selected from —H, halogen, alkyl, heteroalkyl, —NO₂, aryl, heteroaryl, arylalkyl, heteroarylalkyl, and linking group (LG).

The present disclosure further relates to a compound as disclosed herein attached to a solid support.

The present disclosure also relates to an oligonucleotide probe, comprising a fluorophore, a quenching compound as disclosed herein, and an oligonucleotide, wherein the fluorophore and the quenching compounds are covalently attached to the oligonucleotide. The present disclosure still further relates to a composition comprising a quenching compound as disclosed herein and a nucleic acid molecule.

Also disclosed herein is a method of detecting or quantifying a target nucleic acid molecule in a sample by polymerase chain reaction (PCR), the method comprising: (i) contacting the sample comprising one or more target nucleic acid molecules with a) at least one oligonucleotide probe having a sequence that is at least partially complementary to the target nucleic acid molecule, where the at least one probe undergoes a detectable change in fluorescence upon amplification of the one or more target nucleic acid molecules; and with b) at least one oligonucleotide primer pair; (ii) incubating the mixture of step (i) with a DNA polymerase under conditions sufficient to amplify one or more target nucleic acid molecules; and (iii) detecting the presence or absence or quantifying the amount of the amplified target nucleic acid molecules by measuring fluorescence of the oligonucleotide probe, wherein the oligonucleotide probe comprises: a) a fluorophore;

b) a quenching compound of the present disclosure; and c) an oligonucleotide linker joining the dye and the quenching compound.

Still further disclosed herein is a conjugate, comprising: a) a fluorescent donor compound, wherein the fluorescent donor compound emits light at a wavelength in the visible or near-infrared region of the electromagnetic spectrum upon excitation at an appropriate wavelength and having an initial fluorescence intensity; b) a quenching acceptor compound, wherein the quenching acceptor compound is a substituted 3-imino-3H-dibenzo[c,h]xanthen-11-amine, and c) a linking compound, wherein the fluorescent donor compound and the quenching acceptor compound are attached to the linking compound, wherein the distance between the donor compound and acceptor compound is such that upon excitation at the appropriate wavelength the initial fluorescence intensity of the fluorescent donor compound is reduced by a detectable amount.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows the quenching efficiency of Compound 3 of the present disclosure with Reporter Dye 1 having excitation maxima at 650 nm and emission maxima at 671 nm (95.5% quenching of Dye 1).

FIG. 2 shows the quenching efficiency of Compound 3 of the present disclosure with Reporter Dye 2 having excitation maxima at 682 nm and emission maxima at 697 nm (91.9% quenching of Dye 2).

FIG. 3 shows the quenching efficiency of Compound 3 of the present disclosure with Reporter Dye 3 having excitation maxima at 699 nm and emission maxima at 722 nm (92.9% quenching).

FIG. 4 shows the stability of Compound 3 of the present disclosure with Reporter Dye 1 and Reporter Dye 2 in a thermocycling process in comparison to the stability of QSY™ 21 Quencher with Reporter Dye 1 and Reporter Dye 2 in a thermocycling process.

FIG. 5 shows the quenching efficiency of Compound 35 of the present disclosure with Reporter Dye 1 (85% quenching of the dye).

FIG. 6 shows the quenching efficiency of Compound 26 of the present disclosure with Reporter Dye 1 (89% quenching of the dye).

### DETAILED DESCRIPTION

Reference will now be made in detail to certain embodiments, examples of which are illustrated in the accompanying drawings. While the disclosure provides illustrated embodiments, it will be understood that they are not intended to limit the present disclosure to those embodiments. On the contrary, the present disclosure is intended to cover all alternatives, modifications, and equivalents, which may be included within the disclosure as defined by the appended claims.

Any section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

Unless otherwise stated, the following terms used in the specification and claims are defined for the purposes of this disclosure and have the following meaning:

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, and the like. As used herein, the term “alkylene” refers to a straight or branched, saturated, aliphatic diradical having the number of carbon atoms indicated. For example, C₁-C₆ alkyl includes, but is not limited to, methylene, ethylene, propylene, butylene, pentylene, hexylene, and the like. It will be appreciated that alkyl and alkylene groups can be optionally substituted with one or more substituents by replacement of one or more hydrogen atoms on the alkyl and alkylene group.

As used herein, the term “alkenyl” refers to either a straight chain or branched hydrocarbon radical having the number of carbon atoms indicated, and having at least one double bond. For example, C₂-C₆ alkenyl, includes, but is not limited to, vinyl, propenyl, isopropenyl, butenyl, isobutenyl, butadienyl, pentenyl, hexadienyl, and the like. As used herein, the term “alkenylene” refers to either a straight chain or branched hydrocarbon diradical having the number of carbon atoms indicated, having at least one double bond. For example, C₂-C₆ alkenyl, includes, but is not limited to, vinyl, propenyl, isopropenyl, butenyl, isobutenyl, butadienyl, pentenyl, hexadienyl, and the like. It will be appreciated that alkenyl and alkenylene groups can be optionally substituted with one or more substituents by replacement of one or more hydrogen atoms on the alkenyl and alkenylene group.

As used herein, the term “alkoxy” refers to alkyl radical with the inclusion of at least one oxygen atom within the alkyl chain or at the terminus of the alkyl chain, for example, methoxy, ethoxy, and the like. “Halo-substituted-alkoxy” refers to an alkoxy where at least one hydrogen atom is substituted with a halogen atom. For example, halo-substituted-alkoxy includes trifluoromethoxy, and the like. As used herein, the term “oxy-alkylene” refers to alkyl diradical with the inclusion of an oxygen atom, for example, —OCH₂, —OCH₂CH₂—, —OC₁-C₁₀ alkylene-, —C₁-C₆ alkylene-O—C₁-C₆ alkylene-, poly(alkylene glycol), poly(ethylene glycol) (or PEG), and the like. “Halo-substituted-oxy-alkylene” refers to an oxy-alkylene where at least one hydrogen atom is substituted with a halogen atom. It will be appreciated that alkoxy and oxy-alkylene groups can be optionally substituted with one or more substituents by replacement of one or more hydrogen atoms on the alkoxy and oxy-alkylene group.

As used herein, the term “alkynyl” refers to either a straight chain or branched hydrocarbon radical having the number of carbon atoms indicated, and having at least one triple bond. For example, C₂-C₆ alkynyl, includes, but is not limited to, acetylenyl, propynyl, butynyl, and the like. As used herein, the term “alkynylene” refers to either a straight chain or branched hydrocarbon diradical having the number of carbon atoms indicated, and having at least one triple bond. Examples of alkynylene groups include, but are not limited to, —C≡C—, —C≡CCH₂—, —C≡CCH₂CH₂—, —CH₂C≡CCH₂—, and the like. It will be appreciated that alkynyl and alkynylene groups can be optionally substituted with one or more substituents by replacement of one or more hydrogen atoms on the alkynyl and alkynylene group.

As used herein, the term “aryl” refers to a cyclic hydrocarbon radical having the number of carbon atoms indicated, and having a fully conjugated π-electron system. For example, C₆-C₁₀ aryl, includes, but is not limited to, phenyl, naphthyl, and the like. As used herein, the term “arylene” refers to a cyclic hydrocarbon diradical having the number of carbon atoms indicated, and having a fully conjugated π-electron system. For example, C₆-C₁₀ arylene, includes, but is not limited to, phenylene, naphthylene, and the like. It will be appreciated that aryl and arylene groups can be optionally substituted with one or more substituents by replacement of one or more hydrogen atoms on the aryl and arylene group.

“Heteroalkyl,” Heteroalkanyl,” Heteroalkenyl,” Heteroalkynyl,” Heteroalkyldiyl” and “Heteroalkylene,” by themselves or as part of another substituent, refer to alkyl, alkanyl, alkenyl, alkynyl, alkyldiyl and alkylene groups, respectively, in which one or more of the carbon atoms are each independently replaced with the same or different heteroatoms or heteroatomic groups. Typical heteroatoms and/or heteroatomic groups which can replace the carbon atoms include, but are not limited to, —O—, —S—, —S—O—, —NR′—, —PH—, —S(O)—, —SO₂—, —S(O)NR′—, —SO₂NR′—, and the like, including combinations thereof, where R′ is hydrogen or a substitutents, such as, for example, (C1-C8) alkyl, (C6-C14) aryl or (C7-C20) arylalkyl.

“Cycloalkyl” and “Heterocycloalkyl,” by themselves or as part of another substituent, refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl e.g., morpholin-3-yl, morpholin-4-yl, etc.), piperazinyl (e.g., piperazin-1-yl, piperazin-2-yl, etc.), and the like.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, tetrahydronaphthalene, triphenylene, trinaphthalene, and the like.

“Arylalkyl,” by itself or as part of another substituent, refers to an acyclic alkyl group in which one of the hydrogen atoms bonded to a carbon atom, in some embodiments a terminal or sp carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where alkyl moieties having a specified degree of saturation are intended, the nomenclature arylalkanyl, arylalkenyl and/or arylalkynyl is used. When a defined number of carbon atoms are stated, for example, (C7-C20) arylalkyl, the number refers to the total number of carbon atoms comprising the arylalkyl group.

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), SO₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl,” by itself or as part of another substituent, refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodioxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof.

“Heteroarylalkyl,” by itself or as part of another substituent, refers to an acyclic alkyl group in which one of the hydrogen atoms bonded to a carbon atom, in some embodiments a terminal or sp³ carbon atom, is replaced with a heteroaryl group. Where alkyl moieties having a specified degree of saturation are intended, the nomenclature heteroarylalkanyl, heteroarylalkenyl and/or heteroarylalkynyl is used. When a defined number of atoms are stated, for example, 6-20-membered heteroarylalkyl, the number refers to the total number of atoms comprising the arylalkyl group.

“Haloalkyl,” by itself or as part of another substituent, refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

As used here, the term “sulfo” refers to a sulfonic acid, or salt of sulfonic acid (sulfonate).

As used here, the term “carboxy” refers to a carboxylic acid or salt of carboxylic acid.

As used here, the term “phosphate,” refers to an ester of phosphoric acid, and includes salts of phosphate.

As used here, the term “phosphonate,” refers to a phosphonic acid and includes salts of phosphonate.

As used herein, unless otherwise specified, the alkyl portions of substituents such as alkyl, alkoxy, arylalkyl, alkylamino, dialkylamino, trialkylammonium, or perfluoroalkyl are optionally saturated, unsaturated, linear or branched, and all alkyl, alkoxy, alkylamino, and dialkylamino substituents may be optionally substituted by carboxy, sulfo, amino, or hydroxy.

As used herein, “substituted” refers to a molecule wherein one or more hydrogen atoms are replaced with one or more non-hydrogen atoms, functional groups or moieties. Exemplary substituents include but are not limited to halogen, e.g., fluorine and chlorine, C₁-C₈ alkyl, C₆-C₁₄ aryl, heterocycle, sulfate, sulfonate, sulfone, amino, ammonium, amido, nitrile, nitro, lower alkoxy, phenoxy, aromatic, phenyl, polycyclic aromatic, heterocycle, water-solubilizing group, linkage, and linking moiety. In some embodiments, substituents include, but are not limited to, —X, —R, —OH, —OR, —SR, —SH, —NH₂, —NHR, —NR₂, —NR₃⁺, —N═NR₂, —CX₃, —CN, —OCN, —SCN, —NCO, —NCS, —NO, —NO₂, —N₂⁺, —N₃, —NHC(O)R, —C(O)R, —C(O)NR₂, —S(O)₂O—, —S(O)₂R, —OS(O)₂OR, —S(O)₂NR, —S(O)R, —OP(O)(OR)₂, —P(O)(OR)₂, —P(O)(O⁻)₂, —P(O)(OH)₂, —C(O)R, —C(O)X, —C(S)R, —C(O)OR, —CO₂—, —C(S)OR, —C(O)SR, —C(S)SR, —C(O)NR₂, —C(S)NR₂, —C(NR)NR₂, where each X is independently a halogen and each R is independently —H, C₁-C₆ alkyl, C₆-C₁₄ aryl, heterocycle, or linking group.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

The compounds disclosed herein may exist in unsolvated forms as well as solvated forms, including hydrated forms. In some embodiments, the compounds disclosed herein are soluble in an aqueous medium (e.g., water or a buffer). For example, the compounds can include substituents (e.g., water-solubilizing groups) that render the compound soluble in the aqueous medium. Compounds that are soluble in an aqueous medium are referred to herein as “water-soluble” compounds. Such water-soluble compounds are particularly useful in biological assays. These compounds may exist in multiple crystalline or amorphous forms. In general, all physical forms are equivalent for the uses described herein and are intended to be within the scope of the present disclosure. The compounds disclosed herein may possess asymmetric carbon atoms (i.e., chiral centers) or double bonds; the racemates, diastereomers, geometric isomers and individual isomers of the compounds described herein are within the scope of the present disclosure. The compounds described herein may be prepared as a single isomer or as a mixture of isomers.

Where substituent groups are specified by their conventional chemical formulae and are written from left to right, they equally encompass the chemically identical substituents, which would result from writing the structure from right to left, e.g., —CH₂O— will be understood to also recite —OCH₂—.

It will be understood that the chemical structures that are used to define the compounds disclosed herein are each representations of one of the possible resonance structures by which each given structure can be represented. Further, it will be understood that by definition, resonance structures are merely a graphical representation used by those of skill in the art to represent electron delocalization, and that the present disclosure is not limited in any way by showing one particular resonance structure for any given structure.

Where a disclosed compound includes a conjugated ring system, resonance stabilization may permit a formal electronic charge to be distributed over the entire molecule. While a particular charge may be depicted as localized on a particular ring system, or a particular heteroatom, it is commonly understood that a comparable resonance structure can be drawn in which the charge may be formally localized on an alternative portion of the compound.

The above-defined groups may include prefixes and/or suffixes that are commonly used in the art to create additional well-recognized substituent groups. As non-limiting specific examples, “alkyloxy” and/or “alkoxy” refer to a group of the formula —OR″, “alkylamine” refers to a group of the formula —NHR″ and “dialkylamine” refers to a group of the formula —NR″R″, where each R″ is an alkyl.

As used herein, “energy transfer (ET)” refers to FRET or Dexter energy transfer. As used herein, “FRET” (also referred to as fluorescence resonance energy transfer or Forster resonance energy transfer) refers to a form of molecular energy transfer (MET) by which energy is passed non-radiatively between a donor molecule and an acceptor molecule. Without being bound by theory, it is believed that when two fluorophores whose excitation and emission spectra overlap are in close proximity, excitation of one fluorophore can cause the first fluorophore to transfer its absorbed energy to the second fluorophore, causing the second fluorophore, in turn, to fluoresce. Stated differently, the excited-state energy of the first (donor) fluorophore is transferred by a process sometimes referred to as resonance induced dipole-dipole interaction to the neighboring second (acceptor) fluorophore. As a result, the lifetime of the donor molecule is decreased and its fluorescence is quenched, while the fluorescence intensity of the acceptor molecule is enhanced and depolarized. When the excited-state energy of the donor is transferred to a non-fluorophore acceptor, such as a quencher, the fluorescence of the donor is quenched without subsequent emission of fluorescence by the acceptor. Pairs of molecules that can engage in ET are termed ET pairs. In order for energy transfer to occur, the donor and acceptor molecules must typically be in close proximity (e.g., up to 70 to 100 Angstroms). As used herein, “Dexter energy transfer” refers to a fluorescence quenching mechanism whereby an excitation electron can be transferred from a donor molecule to an acceptor molecule via a non-radiative path. Dexter energy transfer can occur when there is interaction between the donor and acceptor. In some embodiments, the Dexter energy transfer can occur at a distance between the donor and acceptor of about 10 Angstroms or less. In some embodiments, in the Dexter energy transfer, the excited state may be exchanges in a single step. In some embodiments, in the Dexter energy transfer, the excited state mat be exchanges in a two separate steps.

Commonly used methods for detecting nucleic acid amplification products require that the amplified product (i.e., amplicon) be separated from unreacted primers. This is often achieved either through the use of gel electrophoresis, which separates the amplification product from the primers on the basis of a size differential, or through the immobilization of the product, allowing washing away of free primer. Other methods for monitoring the amplification process without separation of the primers from the amplicon, such as for real-time detection, have been described. Some examples include TaqMan® probes, molecular beacons, SYBR GREEN® indicator dye, LUX primers, and others. The principal drawback to intercalator-based detection of PCR product accumulation, such as using SYBR GREEN® indicator dye, is that both specific and nonspecific products generate a signal. Typically, intercalators are used for single-plex detection assays and are not suitable for use for multiplex detection.

Real-time systems for quantitative PCR (qPCR) were improved by the use of probe-based, rather than intercalator-based, PCR product detection. One probe-based method for detection of amplification product without separation from the primers is the 5′ nuclease PCR assay (also referred to as the TaqMan® assay or hydrolysis probe assay). This alternative method provides a real-time method for detecting only specific amplification products. During amplification, annealing of the detector probe, sometimes referred to as a “TaqMan probe” (e.g., 5′nuclease probe) or hydrolysis probe, to its target sequence generates a substrate that is cleaved by the 5′ nuclease activity of a DNA polymerase, such as a Thermus aquaticus (Taq) DNA polymerase, when the enzyme extends from an upstream primer into the region of the probe. This dependence on polymerization ensures that cleavage of the probe occurs only if the target sequence is being amplified.

The term “reporter,” “reporter group” or “reporter moiety” is used in a broad sense herein and refers to any identifiable tag, label, or moiety. In some embodiments, the reporter is a fluorescent reporter moiety or dye.

In general, a TaqMan detector probe can include an oligonucleotide covalently attached to a fluorescent reporter moiety or dye and a quencher moiety or dye. The reporter and quencher dyes are in close proximity, such that the quencher greatly reduces the fluorescence emitted by the reporter dye by FRET. Probe design and synthesis has been simplified by the finding that adequate quenching is typically observed for probes with the reporter at the 5′ end and the quencher at the 3′ end.

During the extension phase of PCR, if the target sequence is present, the detector probe anneals downstream from one of the primer sites and is cleaved by the 5′ nuclease activity of a DNA polymerase possessing such activity, as this primer is extended. The cleavage of the probe separates the reporter dye from quencher dye by releasing them into solution, and thereby increasing the reporter dye signal. Cleavage further removes the probe from the target strand, allowing primer extension to continue to the end of the template strand. Thus, inclusion of the probe does not inhibit the overall PCR process. Additional reporter dye molecules are cleaved from their respective probes with each cycle, affecting an increase in fluorescence intensity proportional to the amount of amplicon produced.

The advantage of fluorogenic detector probes over DNA binding dyes, such as SYBR GREEN®, is that specific hybridization between probe and target is required to generate fluorescent signal. Thus, with fluorogenic detector probes, non-specific amplification due to mis-priming or primer-dimer artifact does not generate a signal. Another advantage of fluorogenic probes is that they can be labeled with different, distinguishable reporter dyes. By using detector probes labeled with different reporters, amplification of multiple distinct sequences can be detected in a single PCR reaction, often referred to as a multiplex assay.

As used herein, the term “probe” or “detector probe” generally refers to any of a variety of signaling molecules indicative of amplification, such as an “oligonucleotide probe.” As used herein, “oligonucleotide probe” refers to an oligomer of synthetic or biologically produced nucleic acids (e.g., DNA or RNA or DNA/RNA hybrid) which, by design or selection, contain specific nucleotide sequences that allow it to hybridize under defined stringencies, specifically (i.e., preferentially) to a target nucleic acid sequence. Thus, some probes or detector probes can be sequence-based (also referred to as “sequence-specific detector probe”), for example 5′ nuclease probes. Various detector probes are known in the art, for example (TaqMan® probes described herein (See also U.S. Pat. No. 5,538,848) various stem-loop molecular beacons (See, e.g., U.S. Pat. Nos. 6,103,476 and 5,925,517 and Tyagi and Kramer, 1996, Nature Biotechnology 14:303-308), stemless or linear beacons (See, e.g., WO 99/21881), PNA Molecular Beacons™ (See, e.g., U.S. Pat. Nos. 6,355,421 and 6,593,091), linear PNA beacons (See, e.g., Kubista et al., 2001, SPIE 4264:53-58), non-FRET probes (See, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can include reporter dyes such as, for example, 6-carboxyfluorescein (6-FAM) or tetrachlorofluorescein (TET) and other dyes known to those of skill in the art. Detector probes can also include quencher moieties such as those described herein, as well as tetramethylrhodamine (TAMRA), Black Hole Quenchers (Biosearch), Iowa Black (IDT), QSY quencher (Molecular Probes), and Dabsyl and Dabcyl sulfonate/carboxylate Quenchers (Epoch). In some embodiments, detector probes can also include a combination of two probes, wherein for example a fluor is on one probe, and a quencher on the other, wherein hybridization of the two probes together on a target quenches the signal, or wherein hybridization on a target alters the signal signature via a change in fluorescence.

As used herein, a “sample” refers to any substance containing, or presumed to contain, one or more biomolecules (e.g., one or more nucleic acid and/or protein target molecules) and can include one or more of cells, a tissue or a fluid extracted and/or isolated from an individual or individuals. Samples may be derived from a mammalian or non-mammalian organism (e.g., including but not limited to a plant, virus, bacteriophage, bacteria, and/or fungus). As used herein, the sample may refer to the substance contained in an individual solution, container, vial, and/or reaction site or may refer to the substance that is partitioned between an array of solutions, containers, vials, and/or reaction sites (e.g., substance partitioned over an array of microtiter plate vials or over an array of array of through-holes or reaction regions of a sample plate; for example, for use in a dPCR assay). In some embodiments, a sample may be a crude sample. For example, the sample may be a crude biological sample that has not undergone any additional sample preparation or isolation. In some embodiments, the sample may be a processed sample that had undergone additional processing steps to further isolate the analyte(s) of interest and/or clean up other debris or contaminants from the sample.

As used herein, the term “amplification” or “amplify” refers to an assay in which the amount or number of one or more target biomolecules is increased, for example, by an amount to allow detection and/or quantification of the one or more target biomolecules. For example, in some embodiments, a PCR assay may be used to amplify a target biomolecule. As used herein, a “polymerase chain reaction” or a “PCR”, unless specifically defined otherwise, refers to either singleplex or multiplex PCR assays, and can be real time or quantitative PCR (wherein detection occurs during amplification) or end-point PCR (when detection occurs at the end of a PCR or after amplification; e.g., a dPCR assay). Other types of assays and methods of amplification or amplifying are also anticipated such as, for example, isothermal nucleic acid amplification and are readily understood by those of skill in the art.

As used herein, the terms “nucleic acid,” “polynucleotide,” and “oligonucleotide” can refer to primers, probes, oligomer fragments to be detected, oligomer controls—either labeled or unlabeled, and unlabeled blocking oligomers and shall be generic to polydeoxyribonucleotides (containing 2-deoxy-D-ribose), polyribonucleotides (containing D-ribose), and any other type of polynucleotide which is an N-glycoside of a purine or pyrimidine base, or modified purine or pyrimidine bases. There is no intended distinction in length between the term “nucleic acid,” “polynucleotide,” and “oligonucleotide,” and these terms will be used interchangeably. “Nucleic acid”, “DNA”, “RNA”, and similar terms can also include nucleic acid analogs. The oligonucleotides, as described herein, are not necessarily physically derived from any existing or natural sequence but may be generated in any manner, including chemical synthesis, DNA replication, reverse transcription or a combination thereof.

The term “analog” or “analogue” includes synthetic analogs having modified base moieties, modified sugar moieties, and/or modified phosphate ester moieties. As used herein, the term “modified base” refers generally to any modification of a base or the chemical linkage of a base in a nucleic acid that differs in structure from that found in a naturally occurring nucleic acid. Such modifications can include changes in the chemical structures of bases or in the chemical linkage of a base in a nucleic acid, or in the backbone structure of the nucleic acid. (See, e.g., Latorra, D. et al., Hum Mut 2003, 2:79-85. Nakiandwe, J. et al., plant Method 2007, 3:2.)

Oligonucleotides described herein, especially those functioning as a probe and/or primer, can include one or more modified bases in addition to the naturally occurring bases adenine, cytosine, guanine, thymine and uracil (represented as A, C, G, T, and U, respectively). In some embodiments, the modified base(s) may increase the difference in the Tₘ between matched and mismatched target sequences and/or decrease mismatch priming efficiency, thereby improving not only assay specificity, but also selectivity. Modified bases can be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. Such modified base(s) may include, for example, 8-Aza-7-deaza-dA (ppA), 8-Aza-7-deaza-dG (ppG), locked nucleic acid (LNA) or 2′-0,4′-C-ethylene nucleic acid (ENA) bases. Other examples of modified bases include, but are not limited to, the general class of base analogues 7-deazapurines and their derivatives and pyrazolopyrimidines and their derivatives (e.g., as described in PCT WO 90/14353, herein incorporated by reference). These base analogues, when present in an oligonucleotide, can strengthen hybridization and improve mismatch discrimination. All tautomeric forms of naturally occurring bases, modified bases and base analogues can be included. Modified internucleotide linkages can also be present in the oligonucleotides described herein. Such modified linkages include, but are not limited to, peptide, phosphate, phosphodiester, phosphotriester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art.

In some embodiments, a modified base is located at (a) the 3-end, (b) the 5′-end, (c) at an internal position, or at any combination of (a), (b) and/or (c) in the oligonucleotide probe and/or primer.

In some embodiments the primer and/or probes as disclosed herein are designed as oligomers that are single-stranded. In some embodiments, the primers and/or probes are linear. In other embodiments, the primers and/or probes are double-stranded or include a double-stranded segment. For example, in some embodiments, the primers and/or probes may form a stem-loop structure, including a loop portion and a stem portion. In some embodiments, the primers and/or probes are short oligonucleotides, having a length of 100 nucleotides or less, more preferably 50 nucleotides or less, still more preferably 30 nucleotides or less and most preferably 20 nucleotides or less with a lower limit being approximately 3-5 nucleotides.

In some embodiments, the Tₘ of the primers and/or probes disclosed herein range from about 50° C. to about 75° C. In some embodiments, the primers and/or probes are between about 55° C. to about 65° C. In some embodiments, the primers and/or probes are between about 60° C. to 70° C. For example, the Tₘ of the primers and/or probes disclosed herein may be 56° C., 57° C., 58° C., 60° C., 61° C., 62° C., 63° C., 64° C., 65° C., 66° C., etc. In some other embodiments, the Tₘ of the primers and/or probes disclosed herein may be 56° C. to 63° C., 58° C. to 68° C. 61° C. to 69° C., 62° C. to 68° C., 63° C. to 67° C., 64° C. to 66° C., or any range in between. In some embodiments, the Tₘ of the primers is lower than the Tₘ of the probes as used herein. In some embodiments the Tₘ of the primers as used herein is from about 55° C. to about 65° C. and the Tₘ of the probes as used herein is from about 60° C. to about 70° C. In some embodiments, the Tₘ range of the primers used in a PCR is about 5° C. to 15° C. lower than the Tₘ range of the probes used in the same PCR. In yet other embodiments, the Tₘ of the primers and/or probes is about 3° C. to 6° C. higher than the anneal/extend temperature in the PCR cycling conditions employed during amplification.

In some embodiments, the probes include a non-extendable blocker moiety at their 3′-ends. In some embodiments, the probes can further include other moieties (including, but not limited to additional non-extendable blocker moieties that are the same or different, quencher moieties, fluorescent moieties, etc) at their 3′-end, 5′-end, and/or any internal position in between. In some embodiments, the non-extendable blocker moiety can be, but is not limited to, an amine (NH₂), biotin, PEG, DPI₃, or PO₄. In some preferred embodiments, the blocker moiety is a minor groove binder (MGB) moiety.

As used herein, the terms “MGB,” “MGB group,” “MGB compound,” or “MBG moiety” refers to a molecule that binds within the minor groove of double stranded DNA. When conjugated to the 3′ end of an oligonucleotide, an MGB group can function as a non-extendable blocker moiety. MGB moieties can also increase the specificity of an oligonucleotide probe and/or primer. In some embodiments, the Tₘ of an oligonucleotide, such the probes as disclosed herein, may be reduced by the inclusion of an MGB moiety. For example, the Tₘ of a probe as disclosed herein which comprises an MGB moiety may range from about 45° C. to 55° C. In some, embodiments, the Tₘ of a probe is reduced by about 10° C. to 20° C. with the inclusion of an MGB moiety in the same probe.

Although a general chemical formula for all known MGB compounds cannot be provided because such compounds have widely varying chemical structures, compounds which are capable of binding in the minor groove of DNA, generally speaking, have a crescent shape three dimensional structure. Most MGB moieties have a strong preference for A-T (adenine and thymine) rich regions of the B form of double stranded DNA. Nevertheless, MGB compounds which would show preference to C-G (cytosine and guanine) rich regions are also theoretically possible. Therefore, oligonucleotides including a radical or moiety derived from minor groove binder molecules having preference for C-G regions are also within the scope of the present disclosure.

Some MGBs are capable of binding within the minor groove of double stranded DNA with an association constant of 10³M⁻¹ or greater. This type of binding can be detected by well-established spectrophotometric methods such as ultraviolet (UV) and nuclear magnetic resonance (NMR) spectroscopy and also by gel electrophoresis. Shifts in UV spectra upon binding of a minor groove binder molecule and NMR spectroscopy utilizing the “Nuclear Overhauser” (NOESY) effect are particularly well known and useful techniques for this purpose. Gel electrophoresis detects binding of an MGB to double stranded DNA or fragment thereof, because upon such binding the mobility of the double stranded DNA changes.

A variety of suitable minor groove binders have been described in the literature. See, for example, Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C.& Wahnert, U. Prog. Biophys. Molec. Bio. 47:31-112 (1986) and Reddy, B. S. P., Dondhi, S. M., and Lown, J. W., Pharmacol. Therap., 84:1-111 (1999) (the disclosures of which are herein incorporated by reference in their entireties). A preferred MGB in accordance with the present disclosure is DPI₃. Synthesis methods and/or sources for such MGBs, some of which may be commercially available, are also well-known in the art. (See, e.g., U.S. Pat. Nos. 5,801,155; 6,492,346; 6,084,102; and 6,727,356, the disclosures of which are incorporated herein by reference in their entireties).

As used herein, the term “MGB blocker probe,” “MBG blocker,” or “MGB probe” is an oligonucleotide sequence and/or probe further attached to a minor groove binder moiety at its 3′ and/or 5′ end. Oligonucleotides conjugated to MGB moieties form extremely stable duplexes with single-stranded and double-stranded DNA targets, thus allowing shorter probes to be used for hybridization based assays. In comparison to unmodified DNA, MGB probes have higher melting temperatures (Tm) and increased specificity, especially when a mismatch is near the MGB region of the hybridized duplex. (See, e.g., Kutyavin, I. V., et al., Nucleic Acids Research, 2000, Vol. 28, No. 2: 655-661).

In some embodiments, the nucleotide units which are incorporated into the oligonucleotides acting as a probe, can include a minor groove binder (MGB) moiety. In some embodiments, such MGB moieties can have a cross-linking function (an alkylating agent) covalently bound to one or more of the bases, through a linking arm. Similarly, modified sugars or sugar analogues can be present in one or more of the nucleotide subunits of an oligonucleotide disclosed herein. Sugar modifications include, but are not limited to, attachment of substituents to the 2′, 3′ and/or 4′ carbon atom of the sugar, different epimeric forms of the sugar, differences in the alpha- or beta-configuration of the glycosidic bond, and other anomeric changes. Sugar moieties include, but are not limited to, pentose, deoxypentose, hexose, deoxyhexose, ribose, deoxyribose, glucose, arabinose, pentofuranose, xylose, lyxose, and cyclopentyl. In some embodiments, the sugar or glycoside portion of some embodiments of oligonucleotides acting as a probe, e.g., one including an MGB moiety, can include deoxyribose, ribose, 2-fluororibose, 2-0 alkyl or alkenylribose where the alkyl group may have 1 to 6 carbons and the alkenyl group 2 to 6 carbons. In some embodiments, the naturally occurring nucleotides and in the herein described modifications and analogs the deoxyribose or ribose moiety can form a furanose ring, and the purine bases can be attached to the sugar moiety via the 9-position, the pyrimidines via the I-position, and the pyrazolopyrimidines via the I-position. And in some embodiments, especially in the oligonucleotides acting as a probe (e.g., the third and/or sixth oligonucleotide, target site-specific probe), the nucleotide units of the oligonucleotides can be interconnected by a “phosphate” backbone, as is well known in the art and/or can include, in addition to the “natural” phosphodiester linkages, phosphorothioates and methylphosphonates. Other types of modified oligonucleotides or modified bases are also contemplated herein as would be understood by those of ordinary skill in the art.

When two different, non-overlapping (or partially overlapping) oligonucleotides anneal to different regions of the same linear complementary nucleic acid sequence, and the 3′ end of one oligonucleotide points toward the 5′ end of the other, the former may be called the “upstream” oligonucleotide and the latter the “downstream” oligonucleotide.

As used herein, the terms “target sequence,” “target nucleic acid,” “target nucleic acid sequence,” and “nucleic acid of interest” are used interchangeably and refer to a desired region of a nucleic acid molecule which is to be either amplified, detected or both.

“Primer” as used herein can refer to more than one primer and refers to an oligonucleotide, whether occurring naturally or produced synthetically, which is capable of acting as a point of initiation of synthesis when placed under conditions in which synthesis of a primer extension product which is complementary to a nucleic acid strand is induced i.e., in the presence of nucleotides and an agent for polymerization such as DNA polymerase, at a suitable temperature for a sufficient amount of time and in the presence of a buffering agent. Such conditions can include, for example, the presence of at least four different deoxyribonucleoside triphosphates (such as G, C, A, and T) and a polymerization-inducing agent such as DNA polymerase or reverse transcriptase, in a suitable buffer (“buffer” includes substituents which are cofactors, or which affect pH, ionic strength, etc.), and at a suitable temperature. In some embodiments, the primer may be single-stranded for maximum efficiency in amplification. The primers herein are selected to be substantially complementary to the different strands of each specific sequence to be amplified. This means that the primers must be sufficiently complementary to hybridize with their respective strands. A non-complementary nucleotide fragment may be attached to the 5′-end of the primer (such as having a “tail”), with the remainder of the primer sequence being complementary, or partially complementary, to the target region of the target nucleic acid. Commonly, the primers are complementary, except when non-complementary nucleotides may be present at a predetermined sequence or sequence range location, such as a primer terminus as described. In some embodiments, such non-complementary “tails” can comprise a universal sequence, for example, a sequence that is common to one or more oligonucleotides. In certain embodiments, the non-complementary fragment or tail may comprise a polynucleotide sequence such as a poly (T) sequence to hybridize, for example, to a polyadenylated oligonucleotide or sequence.

The complement of a nucleic acid sequence as used herein refers to an oligonucleotide which, when aligned with the nucleic acid sequence such that the 5′ end of one sequence is paired with the 3′ end of the other, is in “antiparallel association.” Complementarity need not be perfect; stable duplexes may contain mismatched base pairs or unmatched bases.

Stability of a nucleic acid duplex is measured by the melting temperature, or “Tₘ.” The Tₘ of a particular nucleic acid duplex under specified conditions is the temperature at which half of the base pairs have disassociated.

As used herein, the term “Tₘ” or “melting temperature” of an oligonucleotide refers to the temperature (in degrees Celsius) at which 50% of the molecules in a population of a single-stranded oligonucleotide are hybridized to their complementary sequence and 50% of the molecules in the population are not-hybridized to said complementary sequence. The Tₘ of a primer or probe can be determined empirically by means of a melting curve. In some cases it can also be calculated using formulas well known in the art (See, e.g., Maniatis, T., et al., Molecular cloning: a laboratory manual/Cold Spring Harbor Laboratory, Cold Spring Harbor, N.Y.: 1982).

As used herein, the term “sensitivity” refers to the minimum amount (number of copies or mass) of a template that can be detected by a given assay. As used herein, the term “specificity” refers to the ability of an assay to distinguish between amplification from a matched template versus a mismatched template. Frequently, specificity is expressed as ΔC_{y}=Ct_{mismatch}−Ct_{match}. In some embodiments, improvement in specificity or “specificity improvement” or “fold difference” is expressed as 2^{(ΔCt_condition1-(ΔCT_condition2)}.

As used herein, the term “Ct” or “Ct value” refers to threshold cycle and signifies the cycle of a PCR amplification assay in which signal from a reporter that is indicative of amplicon generation (e.g., fluorescence) first becomes detectable above a background level. In some embodiments, the threshold cycle or “Ct” is the cycle number at which PCR amplification becomes exponential.

The term “complementary to” is used herein in relation to a nucleotide that can base pair with another specific nucleotide. Thus, for example, adenosine is complementary to uridine or thymidine and guanosine is complementary to cytidine.

The term “identical” means that two nucleic acid sequences have the same sequence or a complementary sequence.

“Amplification” as used herein denotes the use of any amplification procedures to increase the concentration of a particular nucleic acid sequence within a mixture of nucleic acid sequences.

“Polymerization”, which may also be referred to as “nucleic acid synthesis”, refers to the process of extending the nucleic acid sequence of a primer through the use of a polymerase and a template nucleic acid.

The term “label” as used herein refers to any atom or molecule which can be used to provide or aid to provide a detectable and/or quantifiable signal, and can be attached to a biomolecule, such as a nucleic acid or protein. Labels may provide signals detectable by fluorescence, radioactivity, colorimetry, gravimetry, magnetism, enzymatic activity or the like.

Labels that provide signals detectable by fluorescence are also referred to herein as “fluorophores” or “fluors” or “fluorescent dyes.” As used herein, the term “dye” refers to a compound that absorbs light or radiation and may or may not emit light. A “fluorescent dye” refers to a molecule that emits the absorbed light to produce an observable detectable signal (e.g., “acceptor dyes”, “donor dyes”, “reporter dyes”, “big dyes”, “energy transfer dyes”, “on-axis dyes”, “off-axis dyes”, and the like

In some embodiments, the term “fluorophore,” “fluor,” or “fluorescent dye” can be applied to a fluorescent dye molecule that is used in a fluorescent energy transfer pairing (e.g., with a donor dye or acceptor dye). A “fluorescent energy transfer conjugate,” as used herein typically includes two or more fluorophores (e.g., a donor dye and acceptor dye) that are covalently attached through a linker and are capable of undergoing a fluorescence energy transfer process under the appropriate conditions.

The term “quencher,” “quencher compound,” “quencher group,” “quencher moiety” or “quencher dye” is used in a broad sense herein and refers to a molecule or moiety capable of suppressing the signal from a reporter molecule, such as a fluorescent dye.

The term “overlapping” as used herein (when used in reference to oligonucleotides) refers to the positioning of two oligonucleotides on its complementary strand of the template nucleic acid. The two oligonucleotides may be overlapping any number of nucleotides of at least 1, for example by 1 to about 40 nucleotides, e.g., about 1 to 10 nucleotides or 1, 2, 3, 4, 5, 6, 7, 8, 9, or 10 nucleotides. In other words, the two template regions hybridized by oligonucleotides may have a common region which is complementary to both the oligonucleotides.

The terms “thermally cycling,” “thermal cycling,” “thermal cycles,” or “thermal cycle” refer to repeated cycles of temperature changes from a total denaturing temperature, to an annealing (or hybridizing) temperature, to an extension temperature, and back to the total denaturing temperature. The terms also refer to repeated cycles of a denaturing temperature and an extension temperature, where the annealing and extension temperatures are combined into one temperature. A total denaturing temperature unwinds all double stranded fragments into single strands. An annealing temperature allows a primer to hybridize or anneal to the complementary sequence of a separated strand of a nucleic acid template. The extension temperature allows the synthesis of a nascent DNA strand of the amplicon. The term “single round of thermal cycling” means one round of denaturing temperature, annealing temperature and extension temperature. In a single round of thermal cycling, for example, there may be internal repeating cycles of an annealing temperature and an extension temperature. For example, a single round of thermal cycling may include a denaturing temperature, an annealing temperature (i.e., first annealing temperature), an extension temperature (i.e., first extension temperature), another annealing temperature (i.e., second annealing temperature), and another extension temperature (i.e., second extension temperature).

The terms “reaction mixture,” “amplification mixture,” or “PCR mixture” as used herein refer to a mixture of components necessary to amplify at least one amplicon from nucleic acid templates. The mixture may comprise nucleotides (dNTPs), a thermostable polymerase, primers, and a plurality of nucleic acid templates. The mixture may further comprise a Tris buffer, a monovalent salt, and/or Mg²⁺. The working concentration range of each component is well known in the art and can be further optimized or formulated to include other reagents and/or components as needed by an ordinary skilled artisan.

The terms “amplified product” or “amplicon” refer to a fragment of a nucleic acid amplified by a polymerase using a pair of primers in an amplification method such as PCR or reverse transcriptase (RT)-PCR.

As defined herein, “5′→3′ nuclease activity” or “5′ to 3′ nuclease activity” or “5′ nuclease activity” refers to that activity of a cleavage reaction including either a 5′ to 3′ nuclease activity traditionally associated with some DNA polymerases, whereby nucleotides are removed from the 5′ end of an oligonucleotide in a sequential manner, (i.e., E. coli DNA polymerase I has this activity whereas the Klenow fragment does not), or a 5′ to 3′ endonuclease activity wherein cleavage occurs to more than one phosphodiester bond (nucleotide) from the -5′ end, or both, or a group of homologous 5′-3′ exonucleases (also known as 5′ nucleases) which trim the bifurcated molecules, the branched DNA structures produced during DNA replication, recombination and repair. In some embodiments, such 5′ nuclease can be used for cleavage of the labeled oligonucleotide probe annealed to target nucleic acid sequence.

As used herein, the term “phosphodiester portion” refers to a linkage comprising at least one —O—P(O)(OH)—O— functional group. It will be appreciated that a phosphodiester portion can include other groups, such as alkyl, alkylene, alkenylene, oxy-alkylene, such as PEG, in addition to one or more —O—P(O)(OH)—O— functional groups. It will be appreciated that the other groups, such as alkyl, alkylene, alkenylene, oxy-alkylene, such as PEG, can be optionally substituted with one or more substituents by replacement of one or more hydrogen atoms on the group.

As used herein, the term “protecting group” or “PG” refers to any group as commonly known to one of ordinary skill in the art that can be introduced into a molecule by chemical modification of a reactive functional group, such as an amine or hydroxyl, to obtain chemoselectivity in a subsequent chemical reaction. It will be appreciated that such protecting groups can be subsequently removed from the functional group at a later point in a synthesis to provide further opportunity for reaction at such functional groups or, in the case of a final product, to unmask such functional group. Protecting groups have been described in, for example, Wuts, P. G. M., Greene, T. W., Greene, T. W., & John Wiley & Sons. (2006). Greene's protective groups in organic synthesis. Hoboken, N.J: Wiley-Interscience. One of skill in the art will readily appreciate the chemical process conditions under which such protecting groups can be installed on a functional group. In the various embodiments described herein, it will be appreciated by a person having ordinary skill in the art that the choice of protecting groups used in the preparation of the energy transfer dye conjugates described herein can be chosen from various alternatives known in the art. It will further be appreciated that a suitable protecting group scheme can be chosen such that the protecting groups used provide an orthogonal protection strategy. As used herein, “orthogonal protection” refers to a protecting group strategy that allows for the protection and deprotection of one or more reactive functional group with a dedicated set of reaction conditions without affecting other protected reactive functional groups or reactive functional groups.

As used herein, “water-solubilizing group” refers to a moiety that increases the solubility of the compounds in aqueous solution. Exemplary water-solubilizing groups include but are not limited to hydrophilic group, as described herein, polyether, polyhydroxyl, boronate, polyethylene glycol, repeating units of ethylene oxide (—(CH₂CH₂O)—), and the like.

As used herein, “hydrophilic group” refers to a substituent that increases the solubility of the compounds in aqueous solution. Exemplary hydrophilic groups include but are not limited to —OH, —O⁻Z⁺, —SH, —S⁻Z⁺, —NH₂, —NR₃⁺Z⁻, —N═NR₂⁺Z⁻, —CN, —OCN, —SCN, —NCO, —NCS, —NO, —NO₂, —N₂⁺, —N₃, —NHC(O)R, —C(O)R, —C(O)NR₂, —S(O)₂O⁻Z⁺, —S(O)₂R, —OS(O)₂OR, —S(O)₂NR, —S(O)R, —OP(O)(OR)₂, —P(O)(OR)₂, —P(O)(O⁻)₂Z⁺, —P(O)(OH)₂, —C(O)R, —C(S)R, —C(O)OH, —C(O)OR, —CO₂⁻Z⁺, —C(S)OR, —C(S)O⁻Z⁺, —C(O)SR, —C(O)S⁻Z⁺, —C(S)SR, —C(S)S⁻Z⁺, —C(O)NR₂, —C(S)NR₂, —C(NR)NR₂, and the like, where R is H, C₁-C₆ alkyl, C₁-C₆ alkylC₆-C₁₀ aryl, or C₆-C₁₀ aryl, and optionally substituted.

As used herein, “reactive functional group” or “reactive group” means a moiety on the compound that is capable of chemically reacting with a functional group on a different compound to form a covalent linkage, i.e., is covalently reactive under suitable reaction conditions, and generally represents a point of attachment for another substance. Typically the reactive group is an electrophile or nucleophile that can form a covalent linkage through exposure to the corresponding functional group that is a nucleophile or electrophile, respectively. In some embodiments, the “reactive functional group” or “reactive group” can be a hydrophilic group or a hydrophilic group that has been activated to be a “reactive functional group” or “reactive group.” In some embodiments, a “reactive functional group” or “reactive group” can be a hydrophilic group such as a C(O)OR group. In some embodiments, a hydrophilic group, such as a —C(O)OH, can be activated by a variety of methods known in the art to become a reactive functional group, such as by reacting the —C(O)OH group with N,N,N′,N′-tetramethyl-O—(N-succinimidyl)uronium tetrafluoroborate (TSTU) to provide the NHS ester moiety —C(O)O—NHS (a.k.a. the active ester).

Alternatively, the reactive group is a photoactivatable group that becomes chemically reactive only after illumination with light of an appropriate wavelength.

Exemplary reactive groups include, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, alkynes (including strained alkynes, such as DIBO and DBCO), azo compounds, azoxy compounds, and nitroso compounds. Reactive functional groups also include those used to prepare bioconjugates, e.g., N-hydroxysuccinimide esters (or succinimidyl esters (SE)), maleimides, sulfodichlorophenyl (SDP) esters, sulfotetrafluorophenyl (STP) esters, tetrafluorophenyl (TFP) esters, pentafluorophenyl (PFP) esters, nitrilotriacetic acids (NTA), aminodextrans, cyclooctyne-amines and the like. Methods to prepare each of these functional groups are well known in the art and their application to or modification for a particular purpose is within the ability of one of skill in the art (see, for example, Sandler and Karo, eds., Organic Functional Group Preparations, Academic Press, San Diego, 1989). Exemplary reactive groups or reactive ligands include NHS esters, phosphoramidites, and other moieties listed in Table 1 below. Nucleotides, nucleosides, and saccharides (e.g., ribosyls and deoxyribosyls) are also considered reactive ligands due to at least their ability to form phosphodiester bonds through enzymatic catalysis. For the avoidance of doubt, saturated alkyl groups are not considered reactive ligands.

As used herein, the term “solid support,” as used herein, refers to a matrix or medium that is substantially insoluble in liquid phases and capable of binding a molecule or particle of interest. Solid supports suitable for use herein include semi-solid supports and are not limited to a specific type of support. Useful solid supports include solid and semi-solid matrixes, such as aerogels and hydrogels, resins, beads, biochips (including thin film coated biochips), microfluidic chip, a silicon chip, multi-well plate (also referred to as a microtitre plate or microplate), array (such as a microarray), membranes, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as SEPHAROSE (GE Healthcare), poly(acrylate), polystyrene, poly(acrylamide), polyol, agarose, agar, cellulose, dextran, starch, FICOLL (GE Healthcare), heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

A hydrolysis probe assay can exploit the 5′ nuclease activity of certain DNA polymerases, such as a Taq DNA polymerase, to cleave a labeled probe during PCR. One specific example of a hydrolysis probe is a TaqMan probe. In some embodiments, the hydrolysis probe contains a reporter dye at the 5′ end of the probe and a quencher dye at the 3′ end of the probe. During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the close proximity of the reporter dye to the quencher dye results in suppression of the reporter fluorescence primarily by Forster-type energy transfer (Forster, 1948; Lakowicz, 1983). During PCR, if the target of interest is present, the probe specifically anneals between the forward and reverse primer sites. The 5′ to 3′ nucleolytic activity of the Taq DNA polymerase cleaves the probe between the reporter and the quencher only if the probe hybridizes to the target. The probe fragments are then displaced from the target, and polymerization of the strand continues. In some embodiments, the 3′ end of the probe is blocked to prevent extension of the probe during PCR. In general, hybridization and cleavage process occurs in sequential cycles and does not interfere with the exponential accumulation of the product.

Without being bound to these parameters, the general guideline for designing TaqMan probes and primers is as follows: design the primers as close as possible to, but without overlapping the probe; the Tₘ of the probe should be about 10° C. higher than the Tₘ of the primers; select the strand that gives the probe more C than G bases; the five nucleotides at the 3′ end of the primer should have no more than two G and/or C bases, and the reaction should be run on the two-step thermal profile with the annealing and extension under the same temperature of 60° C.

The following description of quencher compounds provides general information regarding construction of the compounds and probes described herein. As described herein, the quencher compounds can be covalently bound, optionally through a linker, to form an energy transfer dye pair with a reporter moiety. In some embodiments, a reporter moiety and the quencher compound can be covalently bound to one another through an analyte. In some embodiments the analyte is a probe, such as an oligonucleotide probe.

Disclosed herein are the compounds of Formula (I):

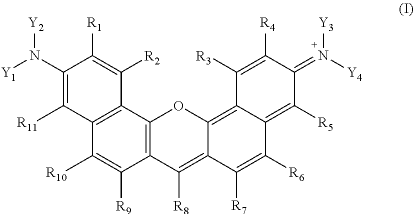

wherein
Y₁ is selected from Y₁′ and —C(O)R″,
Y₂ is selected from Y₂′ and —C(O)R″ on the condition that Y₁ and Y₂ are not both —C(O)R″;
or, alternatively, Y₁ and Y₂ form N═NR′ with the nitrogen to which they are bound;
or, alternatively, Y₁′ forms a saturated or unsaturated, substituted or unsubstituted ring with R₁/R₁₁ together with the atoms to which they are bonded, and/or Y₂′ forms a saturated or unsaturated, substituted or unsubstituted ring with R₁/R₁₁ together with the atoms to which they are bonded;
Y₃ is selected from Y₃′ and —C(O)R″,
Y₄ is selected from Y₄′ and —C(O)R″ on the condition that Y₃ and Y₄ are not both —C(O)R″;
or, alternatively, Y₃′ forms a saturated or unsaturated, substituted or unsubstituted ring with R₄/R₅ together with the atoms to which they are bonded, and/or Y₄′ forms a saturated or unsaturated, substituted or unsubstituted ring with R₄/R₅ together with the atoms to which they are bonded;
R″ is selected from —(CQ¹Q²)ₓ-R^{a};
wherein Q¹ and Q² are independently selected from hydrogen and methyl,
x is an integer ranging from 1 to 10,
R^{a} is a trimethyl quinone;
R₅, R₆, R₇, R₉, R₁₀, R₁₁ are independently selected from —H, halogens, alkyl, and alkyl group independently substituted with one or more Z₂;
R₁, R₂, R₃, R₄, Y₁′, Y₂′, Y₃′, Y₄′, and R′ are independently selected from —H, alkyl, alkyl independently substituted with one or more Z₂, heteroalkyl, heteroalkyl independently substituted with one or more Z₂, aryl, aryl independently substituted with one or more Z₂, heteroaryl, heteroaryl independently substituted with one or more Z₂, arylalkyl, arylalkyl independently substituted with one or more Z₂, heteroarylalkyl, heteroarylalkyl independently substituted with one or more Z₂, halogen, —OS(O)₂OR, —S(O)₂OR, —S(O)₂R, —S(O)₂NR, —S(O)R, —OP(O)O₂RR, —P(O)O₂RR, —C(O)OR, —NO₂, ═NRR, —NRR, —N⁺RRR, —NC(O)R, —C(O)R, —C(O)NRR, —CN, and —OR;
wherein R is independently selected from —H, alkyl, heteroalkyl, aryl, heteroaryl, arylalkyl, and heteroarylalkyl;
wherein Z₂ is selected from —R, halogen, —OS(O)₂OR, —S(O)₂OR, —S(O)₂R, —S(O)₂NR, —S(O)R, —OP(O)O₂RR—P(O)O₂RR, —C(O)OR, —NO₂, —NRR, —N⁺RRR, —NC(O)R, —C(O)R, —C(O)NRR, —CN, —O, —OR, —(CH₂)ₓ—R^{b}, —N(CH₂)ₓ—R^{b};
wherein R^{b} is selected from -halogen, —OH, —OR, —SH, —NH₂, —C(O)O, —C(O)OH, —C(O)NH₂;
R₈ is selected from —H, alkyl, alkyl independently substituted with one or more Z₁, heteroalkyl, heteroalkyl independently substituted with one or more Z₁, aryl, aryl independently substituted with one or more Z₁, heteroaryl, heteroaryl independently substituted with one or more Z₁, arylalkyl, arylalkyl independently substituted with one or more Z₁, heteroarylalkyl, and heteroarylalkyl independently substituted with one or more Z₁; and
Z₁ is selected from the group consisting of, —R*, halogen, —CR*R*R*, —OS(O)₂OR*, —S(O)₂OR*, —SO₃, —S(O)₂R*, —S(O)₂NR*, —S(O)R*, —OP(O)O₂R*R*—P(O)O₂R*R*, —C(O)OR*, —N═N—R*—R*, —NO₂—NR*R*, —N*R*R*R*, —NC(O)R*, —C(O)R*, —C(O)NR*R*, —CN, —O and —OR*, wherein R* is independently selected from —H, halogen, alkyl, heteroalkyl, —NO₂, aryl, heteroaryl, arylalkyl, heteroarylalkyl, and linking group (LG).

In some embodiments of the compounds of Formula (I), Y₁ is selected from Y₁′ and forms a saturated or unsaturated, substituted or unsubstituted ring with R₁₁ together with the atoms to which they are bonded. In at least one embodiment, the ring formed with R₁₁ is unsaturated and substituted. In another embodiment, the ring formed with R₁₁ is saturated and substituted. In another embodiment, the ring formed with R₁₁ is saturated and unsubstituted.

In a preferred embodiment, Y₁ is selected from Y₁′ and Y₂ is selected from Y₂′. In another preferred embodiment, Y₁ is selected from Y₁′ and Y₂ is selected from —H, alkyl and alkyl independently substituted with one or more Z₂. In another preferred embodiment, Y₁ is selected from Y₁′ and forms an unsaturated and substituted ring with R₁₁ together with the atoms to which they are bonded and Y₂ is —H. In another preferred embodiment, Y₁ is selected from Y₁′ and forms a saturated and substituted ring with R₁₁ together with the atoms to which they are bonded and Y₂ is —H. In another preferred embodiment, Y₁ is selected from Y₁′ and forms a saturated and unsubstituted ring with R₁₁ together with the atoms to which they are bonded and Y₂ is —H.

In another preferred embodiment, Y₁ is selected from alkyl, alkyl independently substituted with one or more Z₂ and aryl, and Y₂ is selected from —H, alkyl and alkyl independently substituted with one or more Z₂. In a more preferred embodiment, Y₁ is aryl and Y₂ is —H or alkyl.

In some embodiments of the compounds of Formula (I), Y₄ is selected from Y₄′ and forms a saturated or unsaturated, substituted or unsubstituted ring with R₅ together with the atoms to which they are bonded. In at least one embodiment, the ring formed with R₅ is unsaturated and substituted. In another embodiment, the ring formed with R₅ is saturated and unsubstituted.

In a preferred embodiment, Y₄ is selected from Y₄′ and Y₃ is selected from Y₃′. In another preferred embodiment, Y₄ is selected from Y₄′ and Y₃ is selected from —H, alkyl and alkyl independently substituted with one or more Z₂. In another preferred embodiment, Y₄ is selected from Y₄′ and forms an unsaturated and substituted ring with R₅ together with the atoms to which they are bonded and Y₃ is —H.

In another preferred embodiment, Y₄ is selected from —H, alkyl independently substituted with one or more Z₂ and aryl, and Y₃ is selected from —H, alkyl and alkyl independently substituted with one or more Z₂. In a more preferred embodiment, Y₄ is aryl and Y₃ is —H or alkyl.

In some embodiments of the compounds of Formula (I), Y₁ is selected from Y₁′ and forms a saturated or unsaturated, substituted or unsubstituted ring with R₁₁ together with the atoms to which they are bonded and Y₂ is selected from Y₂′ and forms a saturated or unsaturated, substituted or unsubstituted ring with R₁/R₁₁ together with the atoms to which they are bonded. In a preferred embodiment, the ring formed with R₁₁ is saturated and unsubstituted and the ring formed with R₁ is saturated and unsubstituted.

In some embodiments of the compounds of Formula (I), Y₄ is selected from Y₄′ and forms a saturated or unsaturated, substituted or unsubstituted ring with R₅ together with the atoms to which they are bonded and Y₃ is selected from Y₃′ and forms a saturated or unsaturated, substituted or unsubstituted ring with R₄ together with the atoms to which they are bonded. In a preferred embodiment, the ring formed with R₅ is saturated and unsubstituted and the ring formed with R₄ is saturated and unsubstituted.

In some embodiments of the compounds of Formula (I), Y₁ is selected from Y₁′ and forms a saturated or unsaturated, substituted or unsubstituted ring with R₁₁ together with the atoms to which they are bonded; Y₂ is —H; Y₄ is selected from Y₄′ and forms a saturated or unsaturated, substituted or unsubstituted ring with R₅ together with the atoms to which they are bonded; and Y₃ is —H.

In some embodiments of the compounds of Formula (I), Y₁ is selected from Y₁′ and forms a saturated or unsaturated, substituted or unsubstituted ring with R₁₁ together with the atoms to which they are bonded; Y₂ is selected from Y₂′ and forms a saturated or unsaturated, substituted or unsubstituted ring with R₁/R₁₁ together with the atoms to which they are bonded; Y₃ is selected from Y₃′ and forms a saturated or unsaturated, substituted or unsubstituted ring with R₄ together with the atoms to which they are bonded; and Y₄ is selected from Y₄′ and forms a saturated or unsaturated, substituted or unsubstituted ring with R₅ together with the atoms to which they are bonded. In a preferred embodiment, the ring formed with R₁₁ is saturated and unsubstituted, the ring formed with R₁ is saturated and unsubstituted, the ring formed with R₄ is saturated and unsubstituted and the ring formed with R₅ is saturated and unsubstituted.

In some embodiments of the compounds of Formula (I), one of Y₁ and Y₂ is selected from —C(O)R″. In a preferred embodiment, Y₁ is selected from —C(O)R″ and Y₂ is —H. In some embodiments of the compounds of Formula (I), one of Y₃ and Y₄ is selected from —C(O)R″. In a preferred embodiment, Y₄ is selected from —C(O)R″ and Y₃ is —H. In at least one embodiment, one of Y₁ and Y₂ is selected from —C(O)R″ and one of Y₃ and Y₄ is selected from —C(O)R″. In a preferred embodiment, Y₁ is selected from —C(O)R″, Y₂ is —H, Y₃ is —H and Y₄ is selected from —C(O)R″. In some embodiments of the compounds of Formula (I), Y₁ and Y₂ form N═NR′ with the nitrogen to which they are bound.

In some embodiments of the compounds of Formula (I), R′ is selected from aryl independently substituted with one or more Z₂. In a preferred embodiment, Z₂ is —NRR or —NO₂.

In some embodiments of the compounds of Formula (I), R₆, R₇, R₉, and R₁₀, are each —H.

In some embodiments of the compounds of Formula (I), R₂ and R₃, are both —H.

In some embodiments of Formula (I), R₁, R₄, R₅ and R₁₁, each independently either form a saturated or unsaturated, substituted or unsubstituted ring with Y₂/Y₃/Y₄/Y₁ together with the atoms to which they are bonded, or are —H.

In some embodiments of the compounds of Formula (I), R₈ is selected from

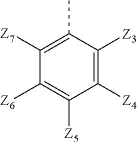

wherein Z₃, Z₄, Z₅ Z₆, and Z₇ each taken separately are independently selected from Z₁. In at least one embodiment, Z₃, Z₄, Z₅, Z₆, and Z₇ are each independently selected from —H, halogen, lower alkyl, —CR*R*R*, —C(O)OR*, —C(O)R*, —S(O)OR*, S(O)₂R*, —SO₃, —N═N—R*—R*, and —CH₂OR*. In at least one embodiment, at least one of Z₃, Z₄, Z₅, Z₆, and Z₇ is —F or —Cl. In at least one embodiment, at least one of Z₃, Z₄, Z₅, Z₆, and Z₇ is —CR*R*R* and R* is F or —Cl. In at least one embodiment, Z₃ is —C(O)OH, —N═N—R*—R*, —R*, —SO₃, —S(O)₂R*, —S(O)₂NR* or CR*R*R*. In a preferred embodiment, Z₃ is C(O)OH. In at least one embodiment, one of Z₅ or Z₆ is —C(O)OH. In at least one embodiment, Z₃ is —S(O)OH and one of Z₅ or Z₆ is —C(O)OH. In at least one embodiment, Z₃ is —C(O)OR* and one of Z₄, Z₅, Z₆, or Z₇ is linking group.

In a preferred embodiment, Z₄ and Z₆ are —H. In another preferred embodiment, Z₃ and Z₇ are each —C(O)OH, —OH, —CR*R*R* or —OR*, and Z₄, Z₅ and Z₆ are each —H. In another preferred embodiment, Z₃ and Z₅ are both —C(O)OH and Z₄, Z₆ and Z₇ are each —H.

In some embodiments of the compounds of Formula (I), R₈ is selected from

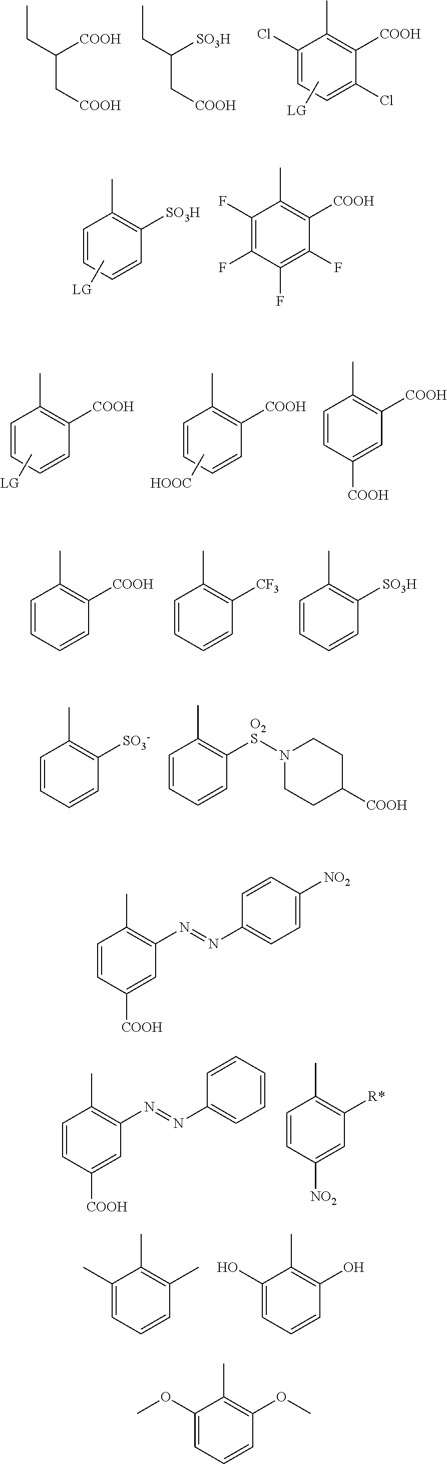

wherein LG is linking group.

In a more preferred embodiment of the compounds of Formula (I), R₈ is selected from

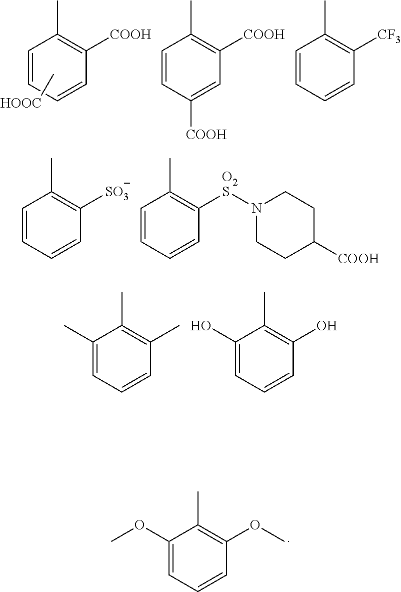

The above-described embodiments of the compounds of Formula (I) can be used in combination with each other. In other words, one, two, three, four, five, six, seven, eight, nine, ten or more of the above described embodiments of the compounds of Formula (I) can be combined with each other so that a substituent such as Y₁, Y₂, Y₃, Y₄, R₁, R₂, R₃, R₄, R₅, R₆, R₇, R₈, R₉, R₁₀ or R₁₁ as defined in one particular embodiment can be combined with one or more of the remaining substituents of Formula (I) as defined in any one of the above-described embodiments.

It is preferred that one or more of the above-described preferred embodiments are combined with each other. Most preferably, all of the above-described preferred embodiments concerning the definition of the substituents Y₁, Y₂, Y₃, Y₄, R₁, R₂, R₃, R₄, R₅, R₆, R₇, R₈, R₉, R₁₀ and R₁₁ of Formula (I) are combined with each other.

In an even more preferred embodiment, the compounds of Formula (I) are represented by the following general Formula (II):

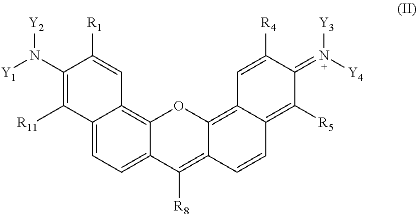

wherein Y₁, Y₂, Y₃, Y₄, R₁, R₄, R₅, R₈ and R₁₁ are as described for Formula (I) or as described in any one of the above-described embodiments. It will be understood that one or more of the above embodiments described for Formula (I) can also be combined with each other for Formula (II). In other words, any one of the substituents Y₁, Y₂, Y₃, Y₄, R₁, R₄, R₅, R₈ or R₁₁ as defined in any one of the above-described embodiments can be combined with one or more of the remaining substituents of Formula (II) as defined in any one of the above-described embodiments. It is preferable that one, two, three, four, five, six or more of the above-described preferred embodiments are combined with each other. Most preferably, all of the above-described preferred embodiments concerning the definition of the substituents Y₁, Y₂, Y₃, Y₄, R₁, R₄, R₅, R₈ or R₁₁ are combined with each other.

The following exemplary and non-limiting Embodiments (1)-(6) of the compounds of Formula (II) are described:

### Embodiment (1)

Y₁, Y₂, Y₃ and Y₄ are selected from Y₁′, Y₂′, Y₃′, Y₄′ and —C(O)R″, respectively, wherein Y₁′, Y₂′, Y₃′, Y₄′ may each independently form an saturated or unsaturated, substituted or unsubstituted ring with R₁₁/R₁/R₄/R₅ together with the atoms to which they are bonded, or Y₁ and Y₂ form N═NR′ with the nitrogen to which they are bound, and/or Y₃ and Y₄ form N═NR′ with the nitrogen to which they are bound, provided that Y₁ and Y₂ are not both —C(O)R″ and provided that Y₃ and Y₄ are not both —C(O)R″;
R₁, R₄, R₅ and R₁₁, each independently either form a saturated or unsaturated, substituted or unsubstituted ring with Y₂/Y₃/Y₄/Y₁ together with the atoms to which they are bonded, or are —H; and
R₈ is selected from

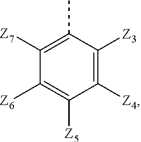

wherein Z₃, Z₄, Z₅, Z₆, and Z₇ are each independently selected from —H, halogen, lower alkyl, —CR*R*R*, —C(O)OR*, —C(O)R*, —S(O)OR*, S(O)₂R*, —SO₃, —N═N—R*—R*, and —CH₂OR*.

### Embodiment (2)

Y₁, Y₂, Y₃ and Y₄ are selected from Y₁′, Y₂′, Y₃′ and Y₄′, respectively, wherein Y₁′, Y₂′, Y₃′, Y₄′ may each independently form an saturated or unsaturated, substituted or unsubstituted ring with R₁₁/R₁/R₄/R₅ together with the atoms to which they are bonded, or Y₁ and Y₂ form N═NR′ with the nitrogen to which they are bound and/or Y₃ and Y₄ form N═NR′ with the nitrogen to which they are bound;
R₁, R₄, R₅ and R₁₁, each independently either form a saturated or unsaturated, substituted or unsubstituted ring with Y₂/Y₃/Y₄/Y₁ together with the atoms to which they are bonded, or are —H; and
R₈ is selected from

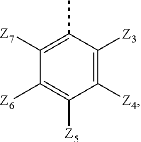

wherein Z₃, Z₄, Z₅, Z₆, and Z₇ are each independently selected from —H, halogen, lower alkyl, —CR*R*R*, —C(O)OR*, —C(O)R*, —S(O)OR*, S(O)₂R*, —SO₃, —N═N—R*—R*, and —CH₂OR*.

### Embodiment (3)

Y₁ and Y₄ are selected from Y₁′ and Y₄′, respectively, wherein Y₁′ and Y₄′ each independently form an saturated or unsaturated, substituted or unsubstituted ring with R₁₁/R₅ together with the atoms to which they are bonded, and Y₂ and Y₃ are each independently selected from —H, alkyl and alkyl independently substituted with one or more Z₂;
R₁ and R₄ are both —H;
R₅ and R₁₁ each independently either form a saturated or unsaturated, substituted or unsubstituted ring with Y₄/Y₁ together with the atoms to which they are bonded; and
R₈ is selected from

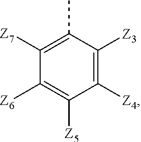

wherein Z₃, Z₅ and Z₇ are each independently selected from —H, halogen, lower alkyl, —CR*R*R*, —C(O)OR*, —C(O)R*, —S(O)OR*, S(O)₂R*, —SO₃, —N═N—R*—R*, and —CH₂OR*; and Z₄ and Z₆ are each —H.

### Embodiment (4)

Y₁, Y₂, Y₃ and Y₄ are selected from Y₁′, Y₂′, Y₃′ and Y₄′, respectively, and Y₁′, Y₂′, Y₃′, Y₄′ each independently form a saturated or unsaturated, substituted or unsubstituted ring with R₁₁/R₁/R₄/R₅ together with the atoms to which they are bonded;
R₁, R₄, R₅ and R₁₁, each independently either form a saturated or unsaturated, substituted or unsubstituted ring with Y₂/Y₃/Y₄/Y₁ together with the atoms to which they are bonded; and
R₈ is selected from

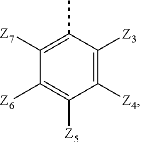

wherein Z₃, Z₅ and Z₇ are each independently selected from —H, halogen, lower alkyl, —CR*R*R*, —C(O)OR*, —C(O)R*, —S(O)OR*, S(O)₂R*, —SO₃, —N═N—R*—R*, and —CH₂OR*; and Z₄ and Z₆ are each —H.

### Embodiment (5)

Y₁ and Y₄ are each independently selected from —H, alkyl independently substituted with one or more Z₂ and aryl; and Y₂ and Y₃ are each independently selected from —H, alkyl and alkyl independently substituted with one or more Z₂;
R₁, R₄, R₅ and R₁₁, each —H; and
R₈ is selected from

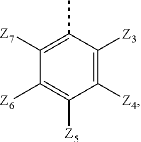

wherein Z₃, Z₅ and Z₇ are each independently selected from —H, halogen, lower alkyl, —CR*R*R*, —C(O)OR*, —C(O)R*, —S(O)OR*, S(O)₂R*, —SO₃, —N═N—R*—R*, and —CH₂OR*; and Z₄ and Z₆ are each —H.

### Embodiment (6)

Y₁, Y₂, Y₃, Y₄, R₁, R₄, R₅ and R₁₁ are defined in the same way as in any one of the above-described Embodiments (1)-(5); and
R₈ is selected from

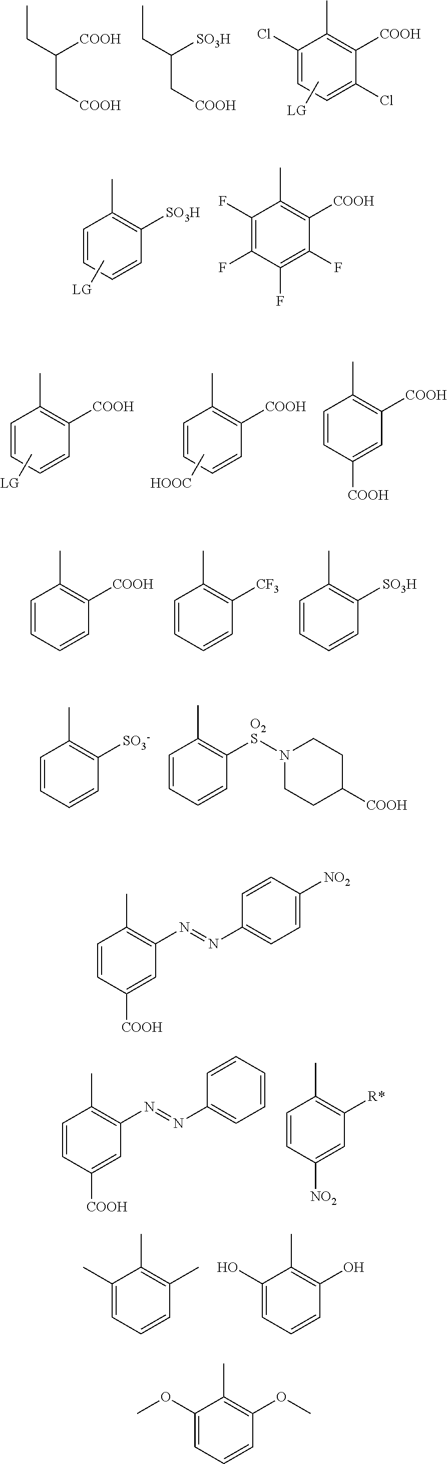

wherein LG is linking group.

As noted, the dibenzoxanthene quenchers of the present disclosure may optionally possess a linking group (LG) comprising at least one group -L₁-Rₓ, where Rₓ is a reactive group that is attached to the dibenzoxanthene compounds by a covalent linkage L₁. In certain embodiments L₁ comprises multiple intervening atoms that serve as a spacer, while in other embodiments L₁ is simply a bond linking Rₓ to the dye. Quenchers having a linking group may be reacted with a wide variety of organic or inorganic substances Sc that contain or are modified to contain functional groups with suitable reactivity, i.e., a complementary functionality -L₂-R_{y}. In certain embodiments L₂ comprises multiple intervening atoms that serve as a spacer, while in other embodiments L₂ is simply a bond linking R_{y} to the substance Sc. Reaction of the linking group and the complementary functionality results in chemical attachment of the quencher to the conjugated substance Sc, represented by D-L-Sc, where L is the linkage formed by the reaction of the linking group and the complementary functionality.

One of R_{y} or Rₓ typically comprise an electrophile, while the other typically comprises a nucleophile, such that the reaction of the electrophile and nucleophile generate a covalent linkage between the dye and the conjugated substance.

Alternatively, one of R_{y} or Rₓ typically comprise a photoactivatable group, and becomes chemically reactive only after illumination with light of an appropriate wavelength.

Selected examples of electrophiles and nucleophile that are useful in linking groups and complementary functionalities are shown in Table 1, where the reaction of an electrophilic group and a nucleophilic group yields a covalent linkage.

TABLE 1
Examples of routes to covalent linkages

| Electrophilic | Nucleophilic | Resulting |
|---|---|---|
| Group | Group | Covalent Linkage |
| activated esters* | amines/anilines | carboxamides |
| aldehydes | amines/anilines | imines |
| aldehydes or ketones | hydrazines | hydrazones |
| aldehydes or ketones | hydroxylamines | oximes |
| anhydrides | alcohols/phenols | esters |
| anhydrides | amines/anilines | carboxamides |
| aziridines | thiols | thioethers |
| boronates | glycols | boronate esters |
| carbodiimides | carboxylic acids | N-acylureas or anhydrides |
| epoxides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | alcohols | ethers |
| sulfonyl halides | amines/anilines | sulfonamides |
| sulfonyl halides | phenols/alcohols | sulfonate esters |
| azide | alkyne | 1,2,3-triazole |

*Activated esters, as understood in the art, generally have the formula —COΩ, where Ω is a good leaving group (e.g. oxysuccinimidyl (—ONC₄H₄O₂) oxysulfosuccinimidyl (—ONC₄H₃O₂—SO₃H), 1-oxybenzotriazoyl (—OC₆H₄N₃); or an aryloxy group or aryloxy substituted one or more times by electron withdrawing substituents such as nitro, fluoro, chloro, cyano, or trifluoromethyl, or combinations thereof, used to form an anhydride or mixed anhydride —OCOR^{a} or —OCNR^{a}NHR^{b}, where R^{a} and R^{b}, whichmay be the same or different, are C₁-C₆ alkyl, C₁-C₆ perfluoroalkyl, or C₁-C₆ alkoxy; or cyclohexyl, 3-dimethylaminopropyl, or N-morpholinoethyl
**Acyl azides can also rearrange to isocyanates.

The covalent linkage L binds the quencher to the conjugated substance Sc either directly (i.e., L is a single bond) or through a combination of stable chemical bonds. For example, L may be alkyleno, alkyleno independently substituted with one or more Z₁, heteroalkyleno, heteroalkyleno independently substituted with one or more Z₁, aryleno, aryleno independently substituted with one or more Z₁, heteroaryleno, and heteroaryleno independently substituted with one or more Z₁.

The group —Rₓ is may be bound to the dye via the linker L₁ at R₁, R₄-R₁₁, or Y₁-Y₄. In an embodiment, —Rₓ can be bound to the dye via the linker L₁ at the position of one of the substituent groups R₁, R₄-R₁₁, or Y₁-Y₄, so as to replace the respective substituent group in Formula (I). In another embodiment, —Rₓ can be bound to the dye via the linker L₁ at the position of one of the substituent groups R₁, R₄-R₁₁, or Y₁-Y₄, so as to be attached to the respective substituent group in Formula (I). In some embodiments, the linking group -L-Rₓ is bound to the dye at R₈, or Y₁-Y₄. In at least one embodiment, the linking group -L-Rₓ is bound to the dye at R₈.

The selection of the linking group used to attach the quencher to the conjugated substance typically depends on the complementary functionality on the substance to be conjugated. The types of complementary functionalities typically present on the conjugated substances Sc include, but are not limited to, amines, thiols, alcohols, phenols, aldehydes, ketones, phosphates, imidazoles, hydrazines, hydroxylamines, disubstituted amines, halides, epoxides, sulfonate esters, purines, pyrimidines, carboxylic acids, or a combination of these groups. A single type of reactive site may be available on the substance (typical for polysaccharides), or a variety of sites may occur (e.g. amines, thiols, alcohols, phenols), as is typical for proteins.

In some embodiments of the compounds of Formula (I) are selected from those of Table Q:

TABLE Q
Exemplary Quenching Dyes

| Compound # | Abs λₘₐₓ | Structure |
|---|---|---|
| 3 | 707 | 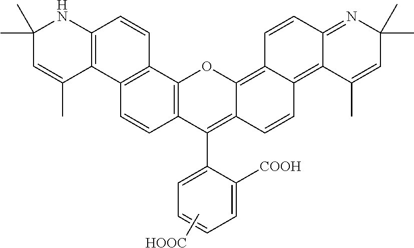 |
| 11 | Not measured | 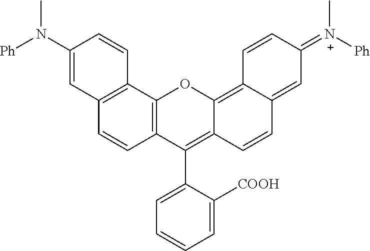 [SMILES: CN(c1ccccc1)c1ccc2c(ccc3c(-c4ccccc4C(=O)O)c4ccc5c/c(=[N+](/C)c6ccccc6)ccc5c4oc32)c1] |
| 17 | 718 | 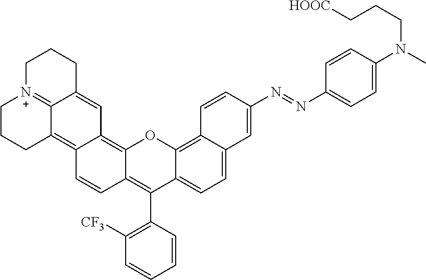 [SMILES: CN(CCCC(=O)O)c1ccc(/N=N/c2ccc3c4c(ccc3c2)C(c2ccccc2C(F)(F)F)=c2ccc3c5c6c(cc3c2O4)CCC[N+]=6CCC5)cc1] |
| 20 | 690 | 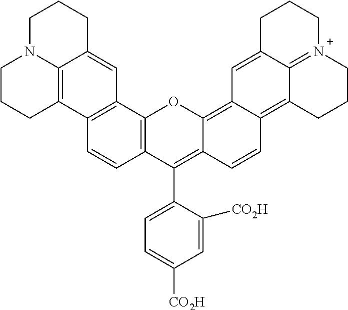 [SMILES: O=C(O)c1ccc(C2=c3ccc4c5c6c(cc4c3Oc3c2ccc2c4c7c(cc32)CCCN7CCC4)CCC[N+]=6CCC5)c(C(=O)O)c1] |
| 26 | 700 | 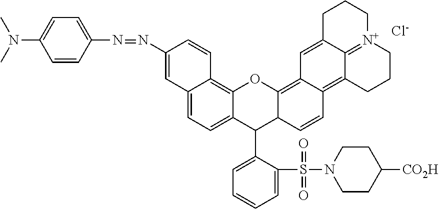 [SMILES: CN(C)c1ccc(N=Nc2ccc3c4c(ccc3c2)C(c2ccccc2S(=O)(=O)N2CCC(C(=O)O)CC2)C2C=Cc3c5c6c(cc3=C2O4)CCC[N+]=6CCC5)cc1 ; [Cl-]] |
| 35 | 646 | 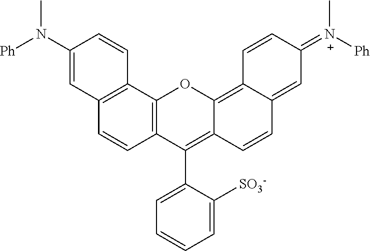 |
| 44 | not measured | 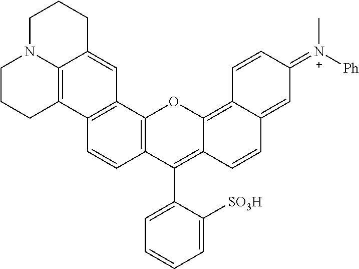 [SMILES: C/[N+](c1ccccc1)=c1\ccc2c(ccc3c(-c4ccccc4S(=O)(=O)O)c4ccc5c6c7c(cc5c4oc32)CCCN7CCC6)c1] |
| 46 | not measured | 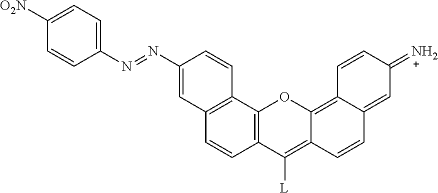 |
| 47 | not measured | 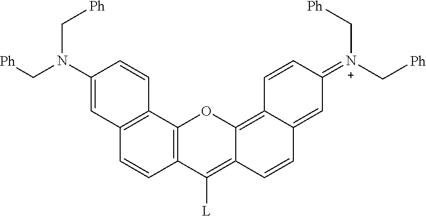 |
| 48 | not measured | 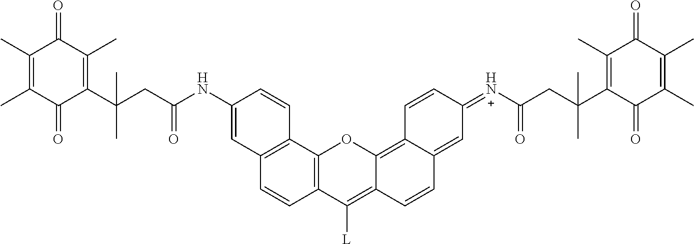 |
| 51 | not measured | 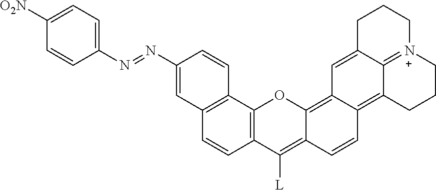 |
| 52 | not measured | 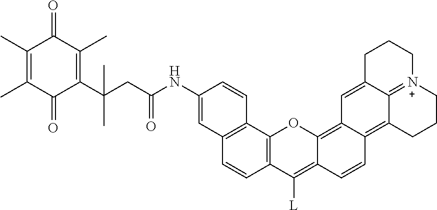 |
| 56 | not measured | 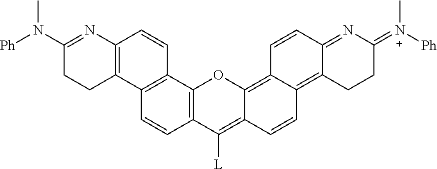 |
| 57 | not measured | 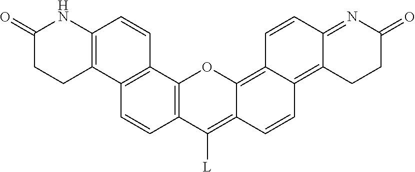 |
| 59 | not measured | 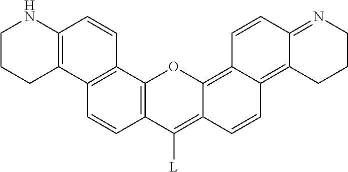 |
| 65 | not measured | 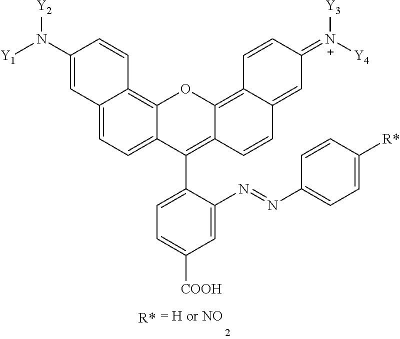 |
| 66 | not measured | 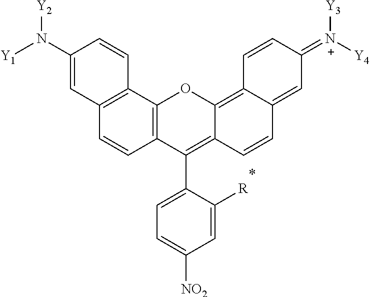 |
| 67 | not measured | 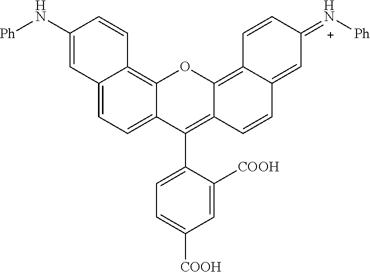 |
| 68 | not measured | 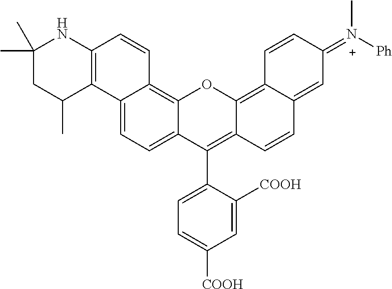 [SMILES: CC1CC(C)(C)Nc2ccc3c(ccc4c(-c5ccc(C(=O)O)cc5C(=O)O)c5ccc6c/c(=[N+](/C)c7ccccc7)ccc6c5oc43)c21] |
| 69 | not measured | 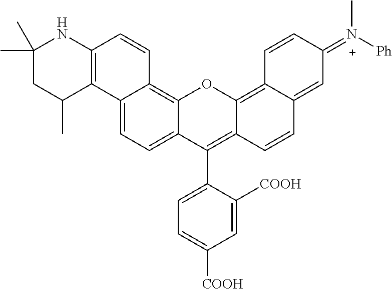 [SMILES: CC1CC(C)(C)Nc2ccc3c(ccc4c(-c5ccc(C(=O)O)cc5C(=O)O)c5ccc6c/c(=[N+](/C)c7ccccc7)ccc6c5oc43)c21] |
| 70 | not measured | 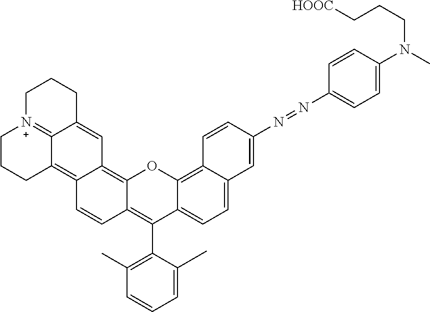 [SMILES: Cc1cccc(C)c1C1=c2ccc3c4c5c(cc3c2Oc2c1ccc1cc(N=Nc3ccc(N(C)CCCC(=O)O)cc3)ccc21)CCC[N+]=5CCC4] |
| 71 | not measured | 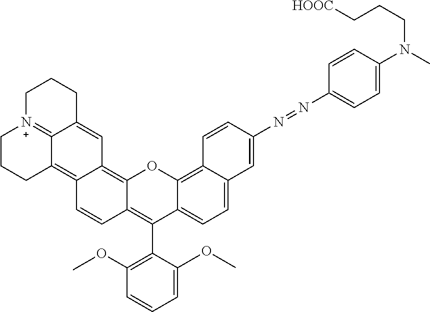 [SMILES: COc1cccc(OC)c1C1=c2ccc3c4c5c(cc3c2Oc2c1ccc1cc(N=Nc3ccc(N(C)CCCC(=O)O)cc3)ccc21)CCC[N+]=5CCC4] |
| 72 | not measured | 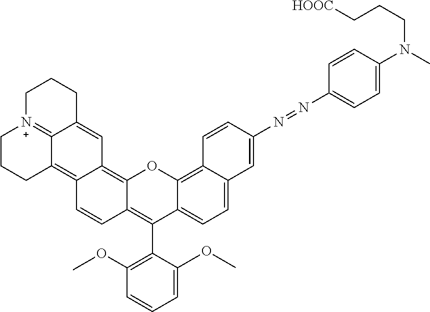 [SMILES: COc1cccc(OC)c1C1=c2ccc3c4c5c(cc3c2Oc2c1ccc1cc(N=Nc3ccc(N(C)CCCC(=O)O)cc3)ccc21)CCC[N+]=5CCC4] |
| 73 | not emasured | 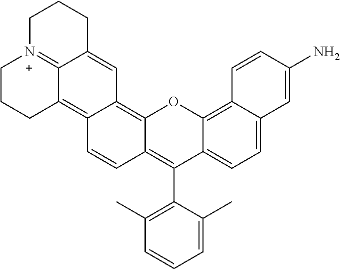 [SMILES: Cc1cccc(C)c1C1=c2ccc3c4c5c(cc3c2Oc2c1ccc1cc(N)ccc21)CCC[N+]=5CCC4] |
| 74 | not measured | 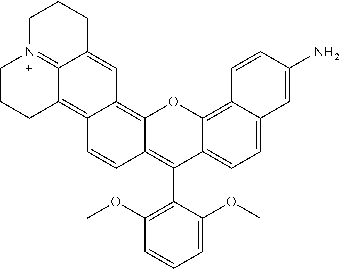 [SMILES: COc1cccc(OC)c1C1=c2ccc3c4c5c(cc3c2Oc2c1ccc1cc(N)ccc21)CCC[N+]=5CCC4] |
| 75 | not measured | 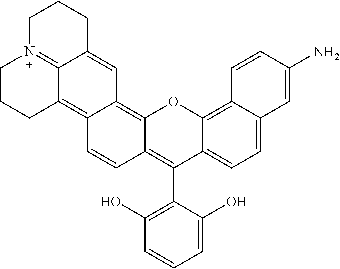 [SMILES: Nc1ccc2c3c(ccc2c1)C(c1c(O)cccc1O)=c1ccc2c4c5c(cc2c1O3)CCC[N+]=5CCC4] |

The compounds as disclosed herein are both thermally and photolytically stable, and are able to quench fluorescence of compounds that emit over a range of wavelengths, preferably over a wavelength range of 600 nm to 800 nm.

In comparison to known cyanine- and rhodamine-based compounds that quench fluorescence in the far-red to near-IR spectral regions, the instant compounds, in particular, the substituted 3-imino-3H-dibenzo[c,h]xanthen-11-amine compounds of Formula (I), are surprisingly thermally stable, such that they can withstand rigorous PCR conditions involving repeated thermocycling steps without significant loss of photophysical properties. The instant compounds also are particularly chemically stable and are well-suited for incorporation into oligonucleotides via automated DNA synthesis without suffering physical degradation (e.g., loss of substituents). The instant compounds are capable of enduring the harsh reaction conditions required for automated oligonucleotide synthesis, which makes them particularly useful in the preparation of oligonucleotides incorporating a terminal or internal quenching compound within the oligonucleotide strand.

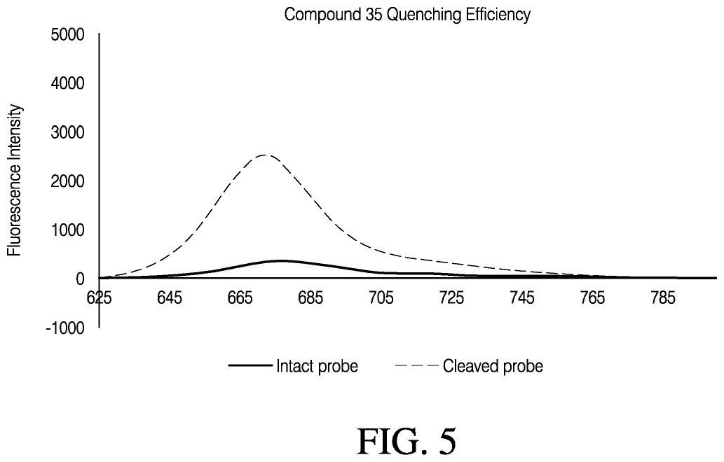

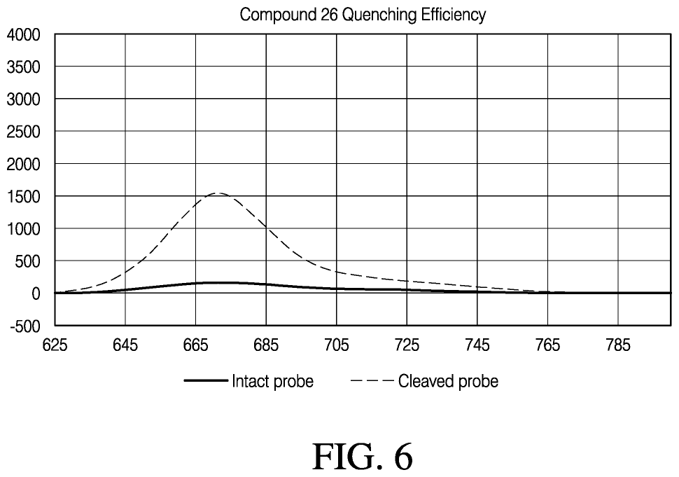

Compounds provided herein that are covalently linked to certain substituents (e.g., azo, nitro, N-phenyl and azide) can be particularly effective at quenching fluorescence of fluorophores that emit in the far-red or near-IR spectral region, such as described herein. For example, compounds bearing an electron rich aniline (N-phenyl) or azo substituent(s) demonstrated significant quenching (i.e., about 85%-90%) of fluorescence emission intensity of partner fluorophores (see FIGS. 5 and 6).

FIG. 5 shows that Compound 35 of the present disclosure has demonstrated a quenching efficiency of 85% with Reporter Dye 1.

FIG. 6 shows that Compound 26 of the present disclosure has a quenching efficiency of 89% with Reporter Dye 1.

The present disclosure also relates to a compound as disclosed herein attached to a solid support.

Some embodiments described herein comprise solid supports to which the other moieties and/or groups of the quenching compounds of Formula (I) are attached. The solid supports are typically activated with functional groups, such as amino or hydroxyl groups, to which linkers bearing linking groups suitable for attachment of the other moieties are attached.

A variety of materials that can be activated with functional groups suitable for attachment to a variety of moieties and linkers, as well as methods of activating the materials to include the functional groups, are known in the art, and include by way of example, controlled pore glass, polystyrene bead and graft co-polymers. Any of these materials be used as solid supports in the embodiments described herein.

Linkers attaching the quenching compounds of Formula (I) to the solid supports typically include linkages that are selectively cleavable under specified conditions such that, following synthesis, the synthesized labeled oligonucleotide can be released from the solid support. In some embodiments, the linkages are labile to the conditions used to deprotect the synthetic labeled oligonucleotide, such that the oligonucleotide is deprotected and cleaved from the solid support in a single step. Such linkers typically include ester linkages, but may include other linkages, such as, for example, carbonate esters, diisopropylsiloxy ethers, modified phosphates esters, etc.

Myriad selectively cleavable linkers useful in the context of oligonucleotide synthesis are known in the art, as are methods of derivatizing solid supports with such linkers.

The quencher or fluorophore can be coupled to the solid support by various types of linkers that are known to those skilled in the art, such as, e.g., linkers described in WO 2022/020722 A1, and U.S. Pat. No. 9,040,674, disulfide linkers and a photocleavable linkers. All of these various linkers can be adapted for use in the solid support reagents described herein.

The present disclosure further relates to an oligonucleotide probe, comprising: a) a fluorophore; and b) a quenching compound of Formula (I) according to the present disclosure; and c) an oligonucleotide, wherein the fluorophore and the quenching compounds are covalently attached to the oligonucleotide. In some embodiments of the present disclosure, the oligonucleotide probe is attached to a solid support.

The oligonucleotide probes described herein can be synthesized according to methods known in the art. For example, in one embodiment, the fluorophore and the quenching compound of Formula (I) are covalently conjugated to the termini of an oligonucleotide using the conjugation chemistries and reactive groups described beforehand. In another example, the quenching compounds of Formula (I) or the fluorophore may be conjugated to a solid support and the oligonucleotide is synthesized from the attached quenching compound or fluorophore using standard oligonucleotide synthesis methods, such as an automated DNA synthesizer, and then the other one of the quenching compound or fluorophore is covalently attached to the terminus of the synthesized oligonucleotide. The quenching compound of Formula (I) or the fluorophore can be coupled to the solid support various types of linkers that are known to those skilled in the art, such as, e.g., linkers described in WO 2022/020722 A1, and U.S. Pat. No. 9,040,674, disulfide linkers and a photocleavable linkers.

In some embodiments, the oligonucleotide includes 4 to 100 nucleotides. In a preferred embodiment, the oligonucleotide incudes 15 to 30 nucleotides. When the oligonucleotide includes 4 to 100 nucleotides, the distance between the fluorophore and the quenching compound is in the range of 13 to 340 Angstrom.

In some embodiments, the present disclosure relates to an oligonucleotide probe composition comprising an oligonucleotide probe as described herein and an aqueous medium.

In at least one embodiment, the oligonucleotide probe composition further comprises a polymerase. In at least one embodiment of the oligonucleotide probe composition the polymerase is a DNA polymerase. In at least one embodiment of the oligonucleotide probe composition the polymerase is thermostable. In at least one embodiment of the oligonucleotide probe composition, the composition further comprises a reverse transcriptase (RT). In at least one embodiment of the oligonucleotide probe composition, the composition further comprises at least one deoxyribonucleoside triphosphate (dNTP).

In at least one embodiment of the oligonucleotide probe composition, the composition further comprises one or more of the following: a) a passive reference control; b) glycerol; c) one or more PCR inhibitor blocking agents; d) a uracil DNA glycosylase; e) a detergent; f) one or more salts; and g) a buffering agent. In at least one embodiment of the oligonucleotide probe composition the one or more salts is a magnesium chloride and/or a potassium chloride.

In at least one embodiment of the oligonucleotide probe composition, the composition further comprises one or more host start components. In at least one embodiment of the oligonucleotide probe composition the one or more hot start components is selected from a chemical modification to the polymerase, oligonucleotide that is inhibitory to the polymerase, and an antibody specific to the polymerase.

In some embodiments of the present disclosure, the oligonucleotide probe composition, further comprises one or more of the following: a) a nucleic acid sample; b) at least one primer oligonucleotide specific for amplification of a target nucleic; and/or c) an amplified nucleic acid product (i.e., an amplicon). In at least one embodiment of the oligonucleotide probe composition the nucleic acid sample is RNA, DNA, or cDNA.

The present disclosure also relates to compositions comprising: a) a quenching compound as disclosed herein; and b) a nucleic acid molecule. In at least one embodiment of the oligonucleotide probe composition, the composition further comprises an enzyme.

In some embodiments, the present disclosure relates to compositions comprising a) a donor fluorophore having an emission spectrum Xd; and b) a quenching compound as disclosed herein having an absorption spectrum of Xq; wherein Xd and Xq overlap in an amount ranging from about 1% to about 100% of the spectrum. In at least one embodiment the fluorophore is or comprises a dye selected from xanthene, coumarin, pyronine, and cyanine dyes. In at least one embodiment, the quenching compounds as disclosed herein have an absorption spectrum Xq in a range between 600 nm and 800 nm. In a more preferred embodiment, the quenching compounds as disclosed herein have an absorption spectrum Xq in a range between 620 nm and 740 nm. In an even more preferred embodiment, the quenching compounds as disclosed herein have an absorption spectrum Xq between about 640 nm to about 720 nm. Thus, the compounds of the present disclosure can quench most effectively in said wavelength ranges.

In some embodiments, the present disclosure relates to a method of detecting or quantifying a target nucleic acid molecule in a sample by polymerase chain reaction (PCR), the method comprising: (i) contacting the sample comprising one or more target nucleic acid molecules with a) at least one oligonucleotide probe having a sequence that is at least partially complementary to the target nucleic acid molecule, where the at least one probe undergoes a detectable change in fluorescence upon amplification of the one or more target nucleic acid molecules; and with b) at least one oligonucleotide primer pair; (ii) incubating the mixture of step (i) with a DNA polymerase under conditions sufficient to amplify one or more target nucleic acid molecules; and (iii) detecting the presence or absence or quantifying the amount of the amplified target nucleic acid molecules by measuring fluorescence of the oligonucleotide probe, wherein the oligonucleotide probe comprises: a) a fluorophore; b) a quenching compound of the present disclosure; and c) an oligonucleotide linker joining the dye and the quenching compound. In at least one embodiment, the PCR is real-time or quantitative PCR (qPCR). In at least one embodiment, the polymerase is a Taq polymerase. In at least one embodiment, the probe is a hydrolysis probe. In at least one embodiment, the target nucleic acid comprises a mutation. In at least one embodiment, the method is used for detection of a rare allele or SNP. In at least one embodiment, the oligonucleotide linker includes 4 to 100 nucleotides. In a preferred embodiment, the oligonucleotide linker incudes 15 to 30 nucleotides. When the oligonucleotide linker includes 4 to 100 nucleotides, the distance between the fluorophore and the quenching compound is in the range of 13 to 340 Angstrom.

In some embodiments, the present disclosure relates to a conjugate, comprising: a) a fluorescent donor compound, wherein the fluorescent donor compound emits light at a wavelength in the visible or near-infrared region of the electromagnetic spectrum upon excitation at an appropriate wavelength and having an initial fluorescence intensity; b) a quenching acceptor compound, wherein the quenching acceptor compound is a substituted 3-imino-3H-dibenzo[c,h]xanthen-11-amine, and c) a linking compound, wherein the fluorescent donor compound and the quenching acceptor compound are attached to the linking compound, wherein the distance between the donor compound and acceptor compound is such that upon excitation at the appropriate wavelength the initial fluorescence intensity of the fluorescent donor compound is reduced by a detectable amount. In at least one embodiment, the quenching acceptor compound is a compound of Formula (I) as described herein. In at least one embodiment, the distance between the donor compound and acceptor compound is in the range of 13 to 340 Angstrom. In a preferred embodiment, the distance between the donor compound and acceptor compound is in the range of 70 to 100 Angstrom.

It should be readily appreciated that the degree of energy transfer, and therefore quenching, is highly dependent upon the separation distance between the reporter moiety (e.g., fluorophore) and the quenching moiety. In molecular systems, a change in fluorescence quenching typically correlates well with a change in the separation distance between the fluorophore molecule and the quenching compound molecule. A fluorophore with sufficient spectral overlap and proximity with a quenching compound is generally a suitable donor for the various applications contemplated herein. The greater the degree of overlap and proximity, the greater the potential for overall quenching.

Quenchers described herein can be used in combination with standard fluorophores. For example, the quencher can be attached to a fluorophore through a linker, such that the quencher and fluorophore are spaced at a distance and orientation for energy transfer to occur under appropriate conditions. Thus, in one example, an energy transfer conjugate is provided where the quencher is attached to an oligonucleotide at one end, and a fluorophore is attached to the opposite end of the strand. In other examples, quencher and fluorophore can be positioned at either 3′ or 5′ terminal ends or either quencher or fluorophore can be positioned at an internal position within the strand. Under the appropriate irradiation conditions, fluorescence emission of the fluorophore in the energy transfer conjugate is diminished by the presence of the quencher in proximity to the fluorophore.

The quenching compounds disclosed herein can have a maximum absorption wavelength between about 640 nm to about 720 nm. Such compounds can be advantageously combined with fluorophores that emit in the wavelength range of about 600 nm to about 800 nm. Thus, also provided herein are fluorophore-quencher pairs that include a quenching compound, as disclosed herein, and a fluorophore that emits between about 600-about 800 nm under appropriate irradiation.

Suitable fluorophores can be any chemical moiety that exhibits an absorption maximum beyond 280 nm when irradiated with light at an appropriate wavelength. Particularly preferred fluorophores for use in combination with the instant quenchers have an absorption maximum of about 500 nm to about 790 nm. In particular embodiments, the absorption maximum of the fluorophore is about 590 nm to about 790 nm.

A wide variety of fluorophores with suitable optical properties for use in combination with the instant compounds are known to one skilled (see, e.g., the MOLECULAR PROBES HANDBOOK: A Guide to Fluorescent Probes and Labeling Technologies by Iain D. Johnson (2010), and the HANDBOOK OF FLUORESCENT PROBES AND RESEARCH PRODUCTS by Richard P. Hagland et al. (2010)).

Exemplary compounds include, without limitation; a pyrene, an anthracene, a naphthalene, an acridine, a stilbene, an indole or benzoindole, an oxazole or benzoxazole, a thiazole or benzothiazole, a 4-amino-7-nitrobenz-2-oxa-1, 3-diazole (NBD), a carbocyanine (including compounds described in U.S. Pat. Nos. 6,403,807; 6,348,599; 5,486,616; 5,268,486; 5,569,587; 5,569,766; 5,627,027 6,048,982; 6,664,047, 6,977,305; and 6,974,873), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and 6,716,979, an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, Exemplary oxazines for use as fluorophores include resorufins (including any corresponding compounds disclosed in U.S. Pat. No. 5,242,805), aminooxazinones, diaminooxazines, and their benzo-substituted analogues.

Representative examples of preferred dyes for use in combination with the quenching compounds described herein include xanthenes (e.g., fluorescein, rhodamine and derivatives thereof). Additional examples of preferred dyes include borapolyazaindacenes, indoles and derivatives thereof.

In certain embodiments, the dye is a xanthene, such as, a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; and 6,562,632). Representative fluorescein compounds includes benzo- or dibenzofluoresceins, seminaphthofluoresceins, or naphthofluoresceins. In certain embodiments, the xanthene dye is a rhodol. Examples of suitable rhodols includes seminaphthorhodafluors (including any corresponding compounds disclosed in U.S. Pat. No. 4,945,171). In certain embodiments, the fluorophore is a fluorinated xanthene dye. Fluorinated xanthenes have been described previously as (i) possessing particularly useful fluorescence properties, such as greater photostability, (ii) having lower sensitivity to pH changes in the physiological range of 6-8 in comparison to non-fluorinated dyes, and (ii) and exhibiting less quenching when conjugated to a substance (Int. Publ. No. WO 97/39064 and U.S. Pat. Nos. 6,162,931 and 6,229,055). In another embodiment, the xanthene dye can be substituted and unsubstituted on the carbon atom of the central ring of the xanthene by substituents typically found in the xanthene-based dyes such as phenyl and substituted-phenyl moieties.

In one aspect the fluorophore contains one or more aromatic or heteroaromatic rings, that are optionally substituted one or more times by a variety of substituents, including without limitation, halogen, nitro, sulfo, cyano, alkyl, perfluoroalkyl, alkoxy, alkenyl, alkynyl, cycloalkyl, arylalkyl, acyl, aryl or heteroaryl ring system, benzo, or other substituents typically present on chromophores or fluorophores known in the art. In one aspect the fluorophore is a xanthene that comprises one or more julolidine rings.

In an exemplary embodiment, the dyes are independently substituted by substituents selected from the group consisting of hydrogen, halogen, amino, substituted amino, alkyl, substituted alkyl, aryl, substituted aryl, heteroaryl, substituted heteroaryl, alkoxy, sulfo. In certain embodiments, the substituent is a reactive group, such as defined above. In other embodiments, the fluorophore is attached to a solid support. For example, the fluorophore can be attached to a solid support, such as a bead for synthesis of an oligonucleotide that includes fluorophore and quencher, as disclosed herein.

In certain embodiments, the quenching compounds described herein can be combined with a cyanine dye. The cyanine dye can emit in the red spectral region upon excitation at an appropriate wavelength. Representative examples of cyanine dyes emitting in the red spectral region include e.g., Alexa Fluor 647, Alexa Fluor 676, DyLight 647, or DyLight 677, available from Thermo Fisher Scientific (Waltham, MA), and derivatives thereof, Cy 5, or Cy 5.5.

In certain embodiments, the cyanine dye is a carbocyanine dye, as described in U.S. Publication No. 2020/407780A1.

The carbocyanine dye may be a modified carbocyanine dye. For instance, these compounds may have at least one substituted indolium ring system wherein the substituent on the 3-carbon of the indolium ring contains a chemically reactive group or a conjugated substance. Other exemplary compounds incorporate an azabenzazolium ring moiety and at least one sulfonate moiety.

In certain cases, fluorophores can include one or more substituents that improve water solubility (e.g., sulfonate and PEG groups). Sulfonated fluorophores include, e.g., sulfonated pyrenes, coumarins, carbocyanines, and xanthenes (as described in U.S. Pat. Nos. 5,132,432; 5,696,157; 5,268,486; and 6,130,101).

The following abbreviations may be relevant for the application.

Abbreviations

| | |
|---|---|
| (D)PBS | (Dulbecco's) phosphate buffered saline |
| (o-Ts)₃P | tris(o-tolyl)phospine |
| abs | absorbance |
| Ac₂O | acetic anhydride |
| ACN | acetonitrile |
| AcOEt | ethylacetate |
| AcOH | acetic acid |
| AcONa | sodium acetate |
| AM | aminomethyl |
| aq | aqueous |
| Ar | argon |
| BINAP | (2,2′-bis(diphenylphosphino)- |
| | 1,1′-binaphthyl |
| cat | catalytic |
| COMU | (1-cyano-2-ethoxy-2- |
| | oxoethylidenaminooxy)dimethylamino- |
| | morpholino-carbenium hexafluorophosphate |
| conc | concentrated |
| D | dimension |
| DBU | 1,8-diazabicyclo[5.4.0]undec-7-ene |
| DCM | dichloromethane |
| DIEA | diethylamine |
| DIPEA | diisopropylethylamine |
| DMA | dimethylacetamide |
| DMF | dimethylformamide |
| DMF•DMA | dimethylformamide/dimethylacetamide |
| DMSO | dimethylsulfoxide |
| DMTr | dimethoxytrityl |
| DSC | differential scanning calorimetry |
| EDC | triethylammonium acetate |
| em | emission |
| eq, equiv | Equivalence |
| Et₂O | diethyl ether |
| Et₃N | triethylamine |
| EtOAc | ethyl acetate |
| EtOH | ethanol |
| h | hour(s) |
| HMBC | heteronuclear multiple bond correlation |
| HOAc | acetic acid |
| HSQC | heteronuclear single quantum coherence |
| IEX | ion-exchange |
| IPA | isopropyl alcohol |
| iPrOAc | isopropylacetate |
| L | liter |
| LCMS | liquid chromatography mass spectrometry |
| M | molar concentration |
| MeCN | acetonitrile |
| MeI | iodomethane |
| MeNPh | N-methylaniline |
| MeOH | methanol |
| MeSO₃H | methanesulfonic acid |
| mg | milligram |
| min | minute(s) |
| mL | milliliter |
| MsOH | methanesulfonic acid |
| MTBE | methyl tert-butyl ether |
| N | normal concentration |
| NaOtBu | sodium tert-butoxide |
| NHS | N-hydroxysuccinimide |
| nm | nanometer |
| NMR | nuclear magnetic resonance |
| ODMT | O-dimethoxytrityl |
| ODU | optical density units |
| PCR | polymerase chain reaction |
| Pd(OAc)₂ | palladium (2) acetate |
| PMT | photomultiplier tube |
| PPA | polyphosphoric acid |
| p-TsOH | p-toluenesulfonic acid |
| Q₃PA | quinone propanoic acid |
| QY | quantum yield |
| RP | reversed phase |
| rt | room temperature |
| sat | saturated |
| T3P | propylphosphonic anhydride |
| TEA | triethylamine |
| TEAA | triethylammonium acetate |
| TFA | trifluoroacetic acid |
| TG | thermogravimetry |
| THF | tetrahydrofuran |
| TLC | thin layer chromatography |
| TSTU | N,N,N′,N′-tetramethyl-O-(N- |
| | succinimidyl)uronium tetrafluoroborate |
| U(H)PLC | ultra (high) performance liquid chromatography |
| vol. | volume |

The following non-limiting examples further describe the compounds, methods, compositions, uses, and embodiments.

### EXAMPLES

### Example 1: Synthesis of Compound 3 Attached to Solid Support (Construct 8)

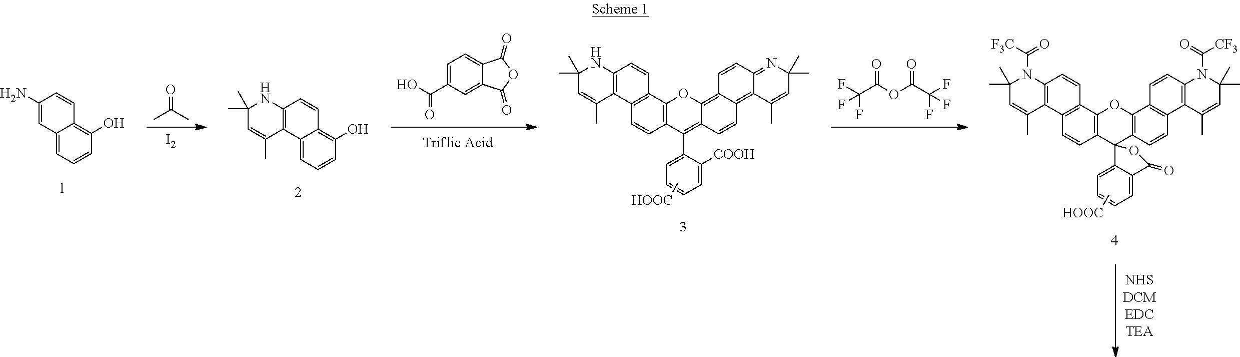

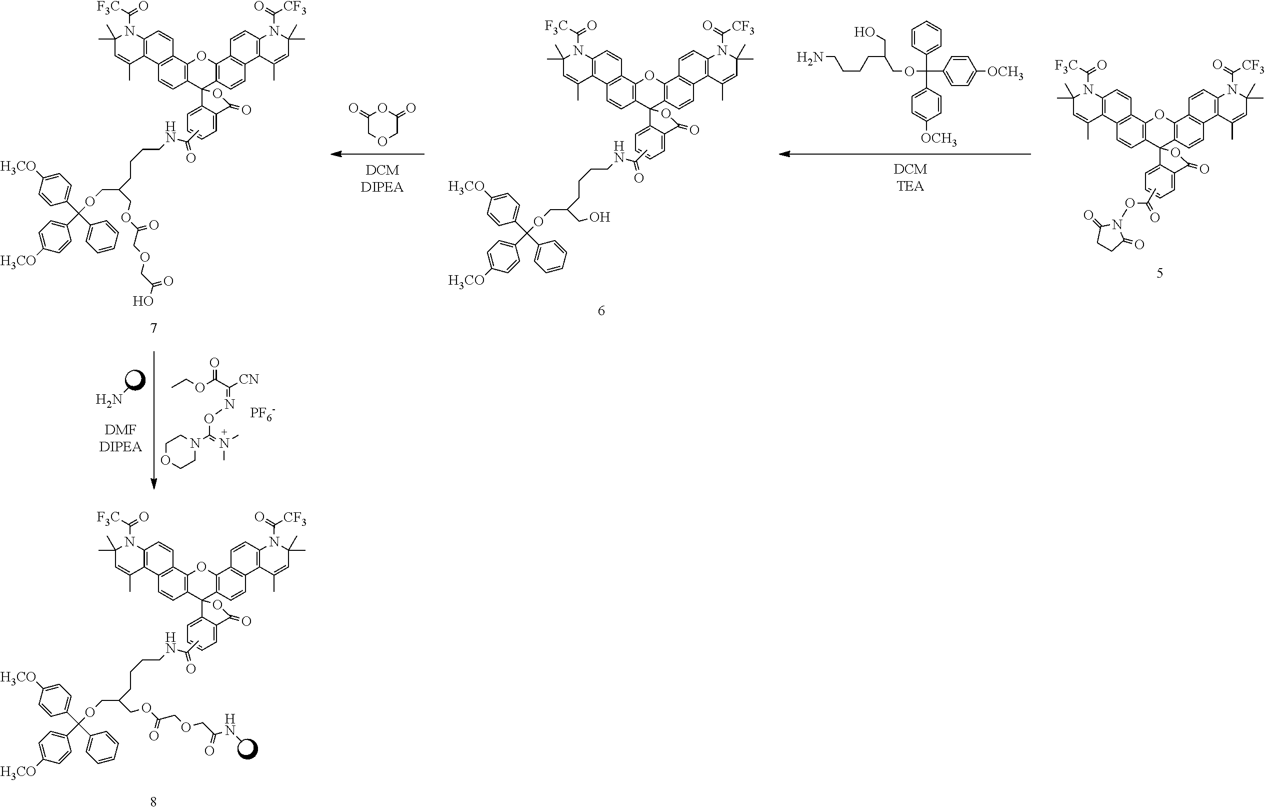

### Example 1.1—Synthesis of 2

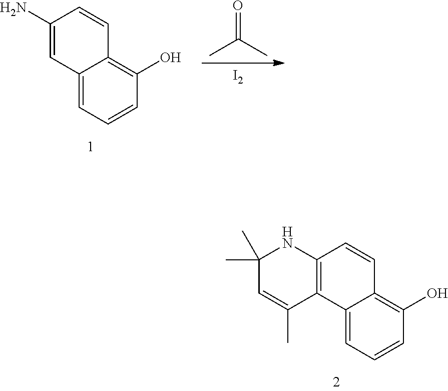

A solution of 6-amino-napthol 1 (0.5 g, 3 mmol), iodine (0.016 g, 0.06 mmol), and acetone (12 mL) was refluxed for 3 h. The reaction was quenched (aqueous Na₂S₂O₃) and extracted with ethyl acetate. The organic extract was dried (Na₂SO₄ powder) and solvent was evaporated. The residue was chromatographed with hexane:ethyl acetate (3:7) to give the product 2 as a yellowish orange solid.

### Example 1.2—Synthesis of 3

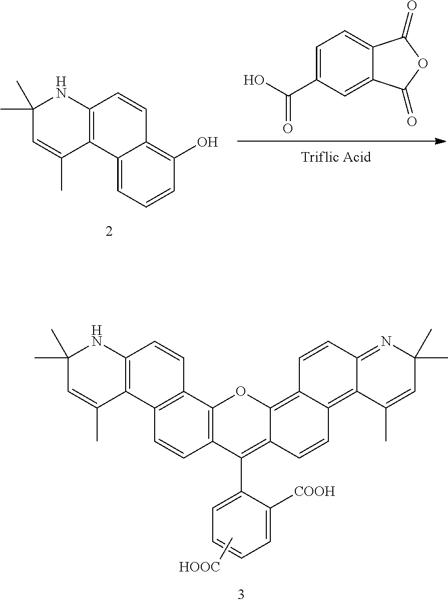

The benzoquinoline 2 (150 mg, 0.62 mmol) and trimellitic anhydride (60 mg, 0.31 mmol) were heated in 4 mL triflic acid at 125° C. for 5 h. The reaction was cooled to room temperature. The acid was neutralized with aqueous sodium hydroxide and the product was extracted with ethyl acetate, dried (Na₂SO₄ powder), and concentrated by rotovap. The residue was chromatographed with DCM/MeOH to give the dye acid as a mixture of 2 isomers 3 (blue solid).

### Example 1.3—Synthesis of 4

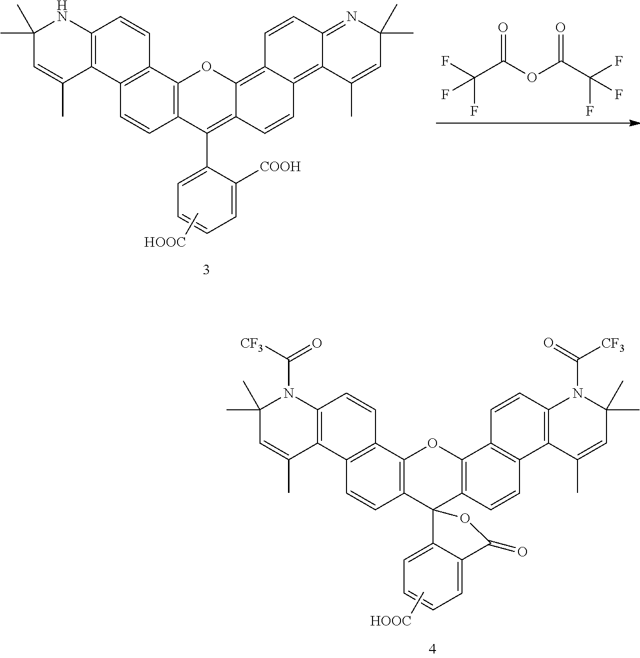

Referring to Scheme 1, the mixture of 2 dye acid isomers 3 (28 mg, 44 μmol) was dissolved in 1 mL dichloromethane. Triethylamine (0.05 mL) was added and the solution was cooled in an ice bath. Trifluoroacetic anhydride (21 μL, 154 μmol) was added and the reaction was stirred for 10 min. The solution was diluted in 12 mL dichloromethane and extracted with 12 mL 1:1 aqueous sodium bicarbonate:brine followed by 12 mL 1:1 1N HCl:brine followed 12 mL brine. The product was dried (Na₂SO₄ powder) and concentrated by rotovap to form the protected dye 4 as a mixture of 2 isomers.

### Example 1.4—Synthesis of 5

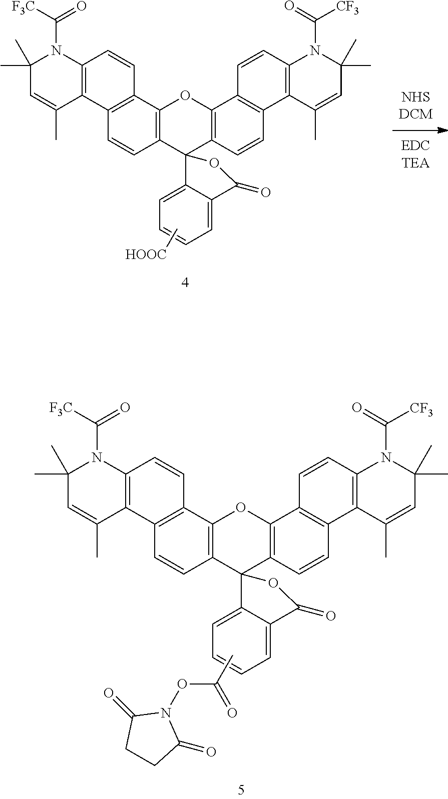

The protected dye isomers 4 (36 mg, 44 μmol) and N-hydroxysuccinimide (10 mg, 87 μmol) were dissolved 1 mL dichloromethane. EDC (12 mg, 74 μmol) was added and the reaction was stirred for 1.5 h. The mixture was diluted in 12 mL dichloromethane and extracted twice with 12 mL 1:1 1N HCl:brine followed 12 mL brine, dried (Na₂SO₄ powder), and concentrated by rotovap. The residue was chromatographed with MeOH/DCM (1:100) to form NHS ester 5 as a mixture of 2 isomers.

### Example 1.5—Synthesis of 6

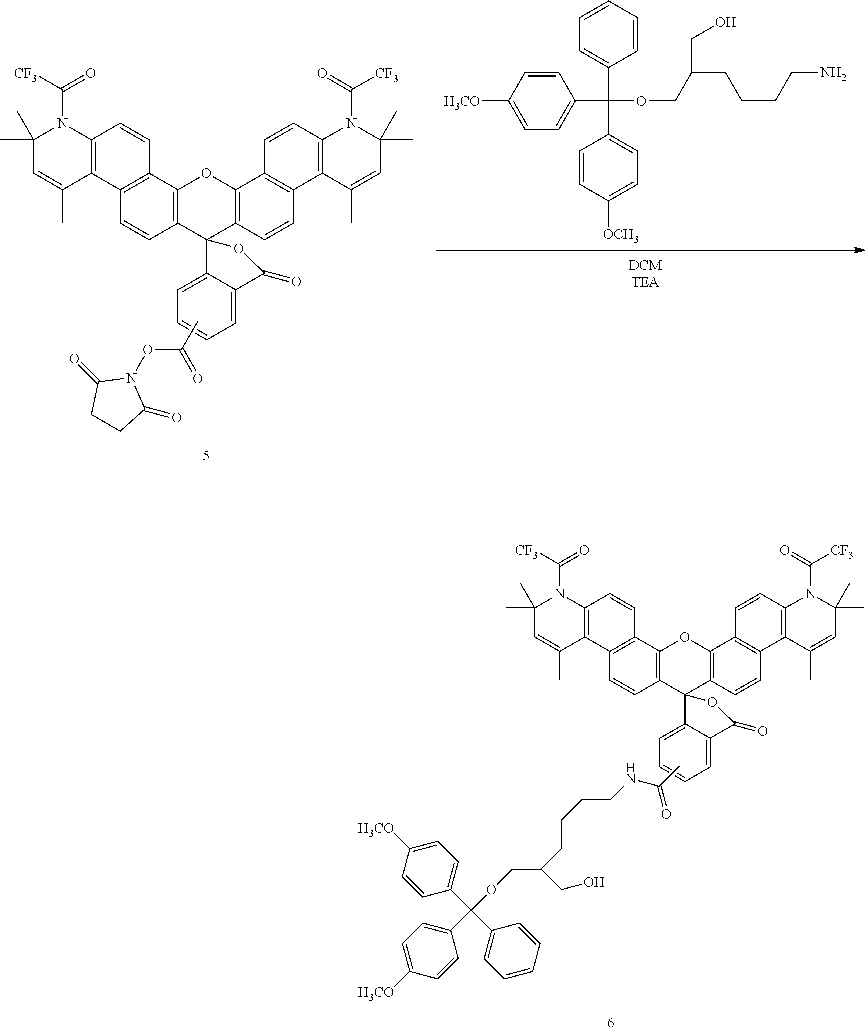

The dye succinimidyl ester isomers 5 (110 mg, 119 μmol) was dissolved in 1 mL dichloromethane. In a test tube, 31 μL diisopropylethylamine was added to 1.2 mL ODMT-aminobutyl-1,3-propanediol. This solution was added to the dye solution. The reaction went to 97% completion in 30 min. The mixture was diluted in 12 mL dichloromethane and extracted twice with 12 mL 1:1 Brine:Water followed 12 mL brine, dried (Na₂SO₄ powder), and concentrated by rotovap. The residue was chromatographed with MeOH/DCM (4:100) to form the dye-E-OH 6 as a mixture of 2 isomers.

### Example 1.6—Synthesis of 7

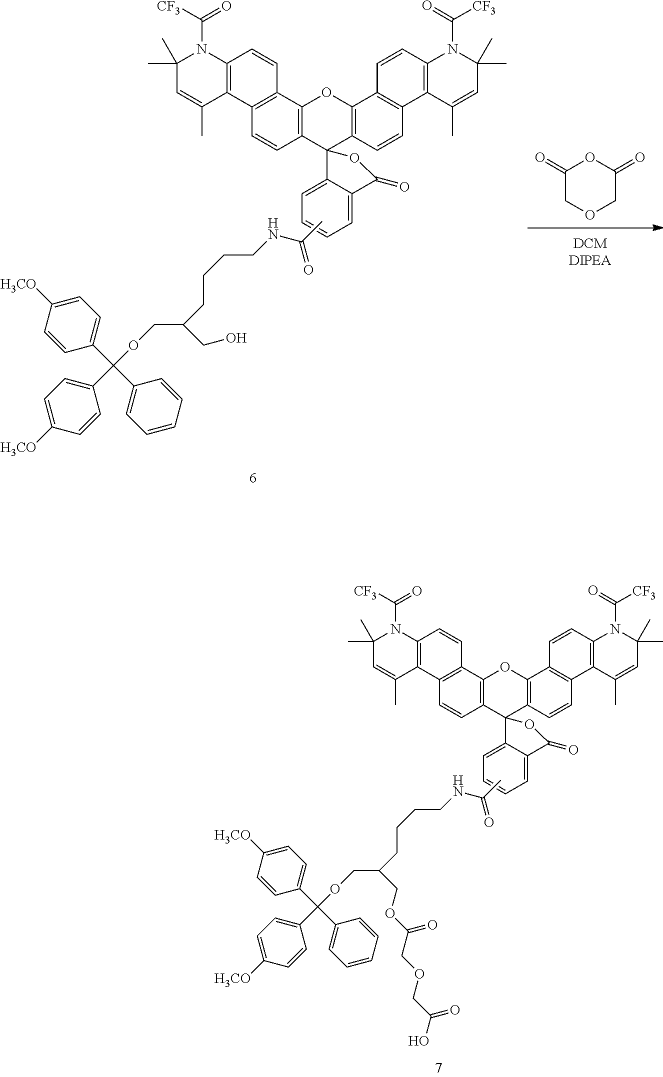

The dye E-OH isomers 6 (173 mg, 137 μmol) was dissolved in 3.4 mL dichloromethane and 60 μL diisopropylethylamine was added to the solution. Diglycolic anhydride (32 mg, 275 μmol) was dissolved in 1 mL DCM and added to the solution with stirring. After 60 min the reaction had gone to 95% completion and the solvent was removed by rotory evaporation to form the dye glycolic linker 7 as a mixture of 2 isomers.

### Example 1.7—Coupling of Compound 7 to Solid Support

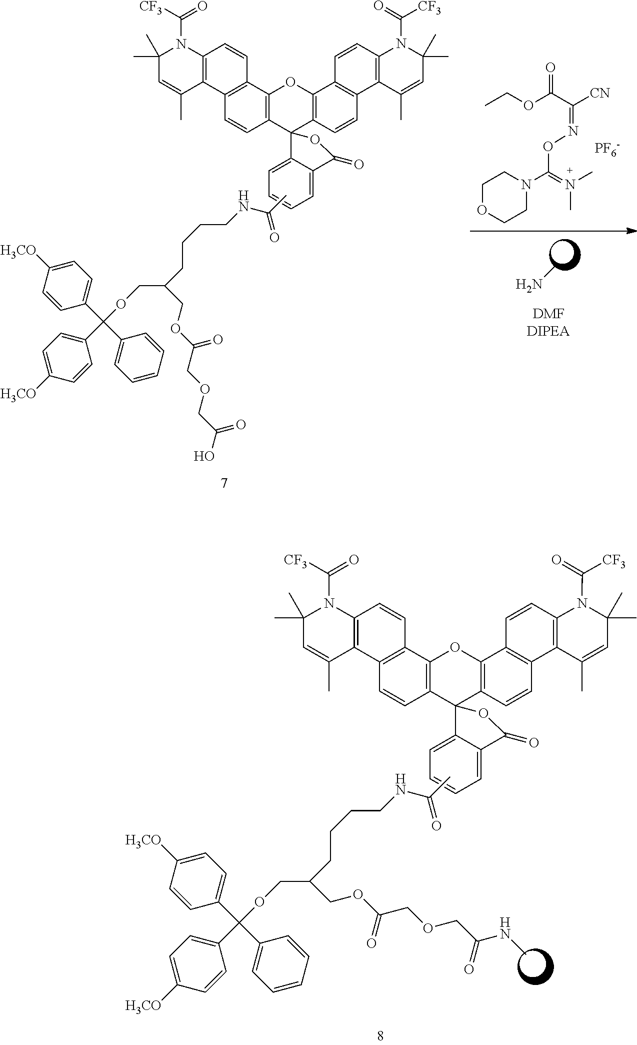

The dye glycolic linker isomers 7 (20 mg, 15 μmol) was dissolved in 3.5 mL dimethylformamide. AM-polystyrene 33 μmol/g (303 mg, 0.01 μmol) was added to the flask followed by 10 μL diisopropylethylamine and then 2-cyano-2-(hydroxyamino)acetate (oxyma) (COMU 13 mg, 30 μmol). The reaction was placed on a shaker. After 3 h the solid was filtered and washed 3 times with 2.5 mL DMF, then 3 times with 2.5 mL acetonitrile, and then 3 times with 2.5 mL dichloromethane. The solid was placed under vacuum to dry overnight.

10.3 mg of the solid was added to a volumetric flask that was then filled with a toluenesulfonic acid in acetonitrile solution. The absorbance was measured at 498 nm and the loading amount of 7 onto the AM-polystyrene support was determined to be 22 μmol/g.

The solid support (270 mg) was added to a flask followed by capping reagents N-methylimidazole/THF (2.5 mL) and acetic anhydride/pyridine/THF (2.5 mL). The flask was placed on a shaker for 1 h. The solid was then filtered and washed 3 times with 2.5 mL THF, followed by 3 times with acetonitrile and then 3 times with dichloromethane. The solid support was dried overnight under high vacuum to provide structure 8.

### Example 2: Synthesis of Compound 11

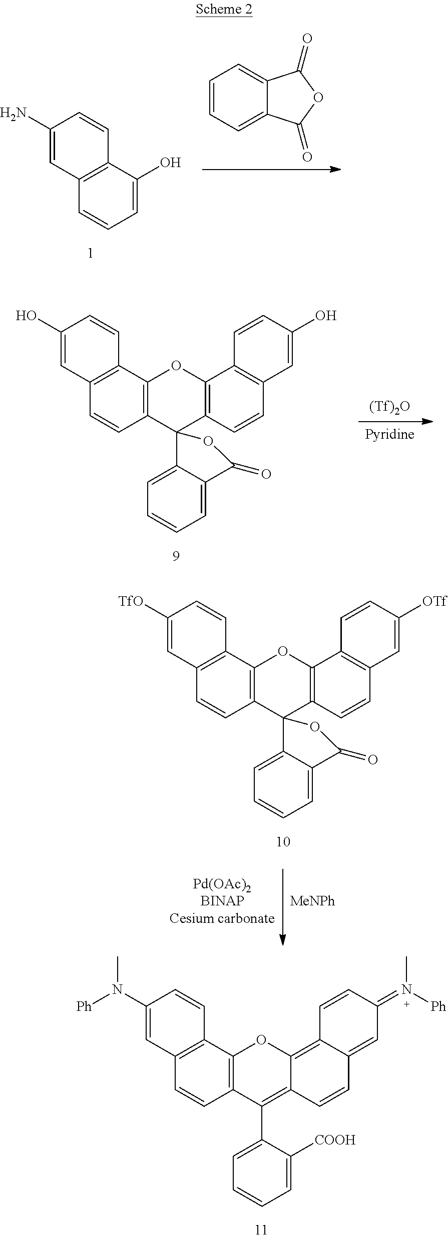

### Example 2.1—Synthesis of 9

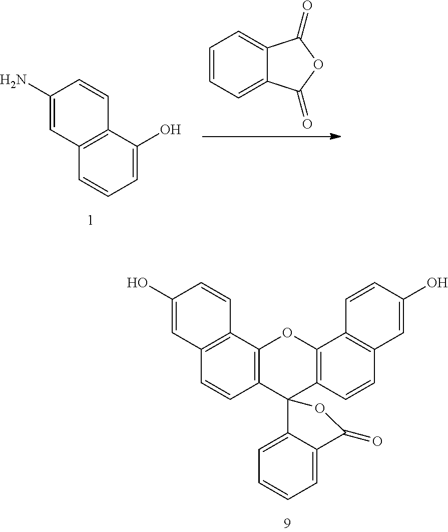 [SMILES: Nc1ccc2c(O)cccc2c1 ; O=C1OC(=O)c2ccccc21 ; O=C1OC2(c3ccccc31)c1ccc3cc(O)ccc3c1Oc1c2ccc2cc(O)ccc12]

6-amino-1-naphthol (1, 1.00 g, 6.24 mmol) and phthalic anyhydride (462 mg, 3.12 mmol) were mixed in 10 mL of methanesulfonic acid and heated at 150° C. for 3 h. The product was precipitated in water and washed until the filtrate was clear and used without further purification.

### Example 2.2—Synthesis of 10

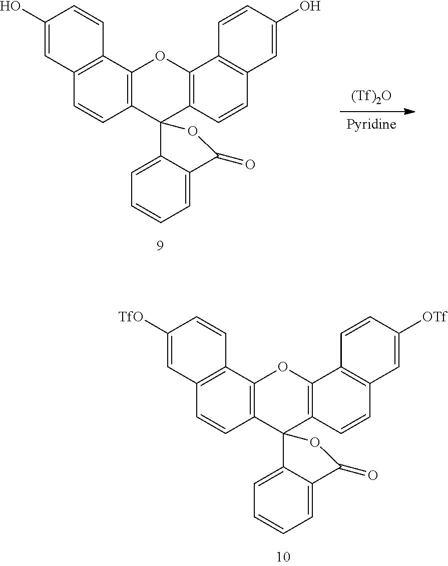 [SMILES: "(Tf)2O" ; O=C1OC2(c3ccccc31)c1ccc3cc(O)ccc3c1Oc1c2ccc2cc(O)ccc12 ; "Pyridine" ; O=C1OC2(c3ccccc31)c1ccc3cc(OS(=O)(=O)C(F)(F)F)ccc3c1Oc1c2ccc2cc(OS(=O)(=O)C(F)(F)F)ccc12]

Naphthofluorescein 9 (400 mg, 0.925 mmol) was suspended in CH₂Cl₂ (5 mL) and cooled to 0° C. Pyridine (600 uL, 7.40 mmol) and trifluoromethanesulfonic anhydride (621 uL, 3.7 mmol) were added to the mixture and the ice bath was removed. The reaction was stirred at room temperature for 4 h and then diluted with water and extracted with CH₂Cl₂ three times. The combined organic extracts were dried with MgSO₄, filtered, and concentrated in vacuo. The triflate 10 was purified by column chromatography on silica gel (0-30% EtOAc/hexanes).

### Example 2.3—Synthesis of 11

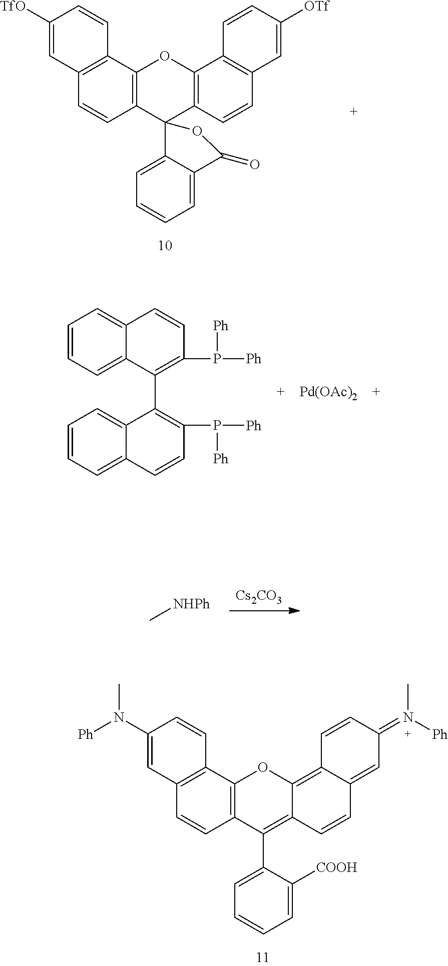

Under nitrogen, 10 (450 mg, 0.64 mmol), BINAP (240 mg, 0.38 mmol), palladium acetate (58 mg, 0.24 mmol) and cesium carbonate (1.18 g, 3.62 mmol mmol) were added to a round bottom flask and the flask was sealed. N-methylaniline (320 μL, 3.0 mmol) was mixed with 4 mL toluene and added to the flask and the reaction was stirred at 100° C. overnight to provide 11.

### Example 3—Synthesis of Compound 18

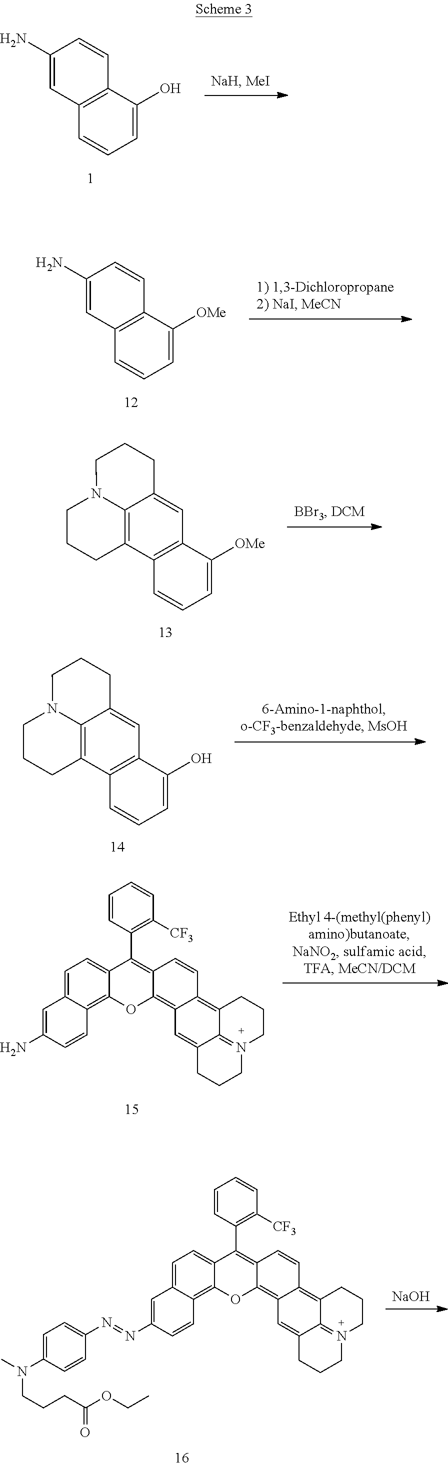

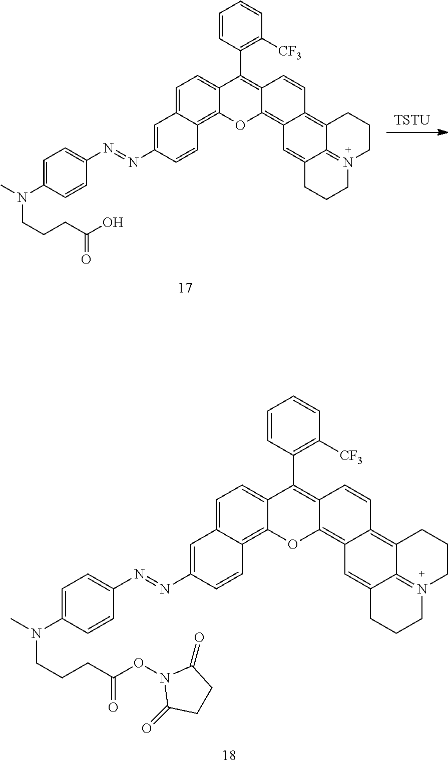

### Example 3.1—Synthesis of 12

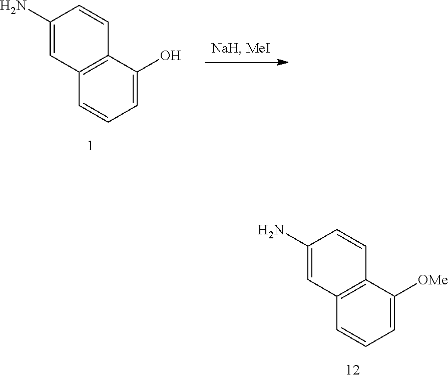

Route A: 6-Amino-1-naphthol (1, 5.16 g, 31.5 mmol) was dissolved in 125 mL DMF in an oven-dried flask. Reaction solution was cooled in ice bath under Ar. 1.39 g NaH (60% in paraffin wax, 34.6 mmol, 1.1 equiv) was added portion-wise through a powder funnel over 5 min and the solution was stirred under Ar at 0° C. for 15 min. Iodomethane (1.96 mL, 31.5 mmol, 1.0 equiv), dissolved in 5 mL DMF, was added to the mixture and the reaction was allowed to stir at rt overnight (20 h). After this time, reaction was quenched with 5 mL H₂O, stirred at rt 5 min, and solvent was removed in vacuo. 300 mL H₂O was added, then product was extracted with Et₂O (2×250 mL), washed with brine (400 mL), dried over MgSO₄, and filtered and concentrated in vacuo. Product was purified by silica column chromatography with 100% DCM to obtain 3.93 g of 12 (72% yield).

Alternative Route A′: 6-Amino-1-naphthol (1, 5.26 g, 33 mmol) was dissolved in 125 mL DMF in an oven-dried flask. Reaction solution was cooled in ice bath under N₂. 1.45 g NaH (60% in paraffin wax, 36 mmol, 1.1 equiv) was added portion-wise through a powder funnel. This was warmed to room temperature. Iodomethane (2.05 mL, 33 mmol, 1 equiv), dissolved in 5 mL DMF, was added to the mixture and the reaction was allowed to stir at rt for 4 h. The reaction was quenched with 5 mL H₂O, stirred at rt for 20 min, and solvent was removed in vacuo. The residue was dissolved in MeOH and adsorbed to silica gel (50 g), dried, and purified by silica column chromatography with 100% DCM then re-columned eluting with 20% EtOAc/hexane to obtain 4.06 g of 12 (71% yield) as a dark amber oil that slowly turned to a brown solid. LCMS expected 179.09, obtained 179.09.

### Example 3.2—Synthesis of 13

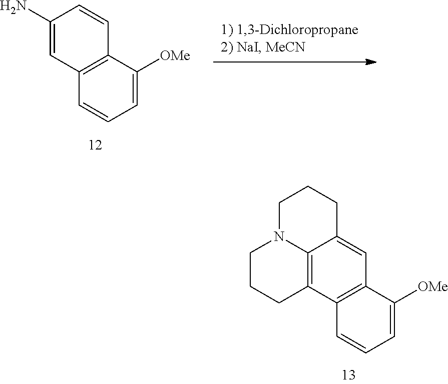

Route A: 12 (94.6 mg, 0.546 mmol) was dissolved in 1,3-dichloropropane (2.0 mL) in a 10 mL microwave reaction vessel with stir bar. Mixture was heated to 250° C. for 30 min in CEM Discover 2.0 microwave twice. Reaction mixture was poured into sat. aq. NaHCO₃ (50 mL), extracted with EtOAc (3×50 mL), washed with brine (50 mL), dried over Na₂SO₄, filtered and concentrated in vacuo. Crude mixture was dissolved in acetonitrile (2.0 mL) in a 10 mL microwave reaction vessel and sodium iodide (327.4 mg, 2.18 mmol, 4.0 equiv) was added. Mixture was heated to 150° C. for 15 min in CEM Discover 2.0 microwave. Reaction mixture was poured into sat. aq. NaHCO₃ (50 mL), extracted with EtOAc (3×50 mL), washed with brine (50 mL), dried over Na₂SO₄, filtered and concentrated in vacuo. Crude product was dissolved in 5 mL 2.0 M dimethylamine in MeOH and was stirred for 20 h, after which time the solvent was removed in vacuo. Product was purified by silica column chromatography (hexanes-7% EtOAc/hexanes) to obtain 42 mg of 13 (30% yield).

Alternative Route A′: 12 (3.65 g, 21.1 mmol) was dissolved in 1-bromo-3-chloropropane (20 mL) and Na₂CO₃ (8.93 g, 84.2 mmol) was added. The mixture was refluxed for 17 h. The mixture was diluted with excess DMF, filtered, and the solid was washed once with more DMF. The filtrate was concentrated, and the residue was dissolved in 50 mL dry DMF. 8.9 g (84 mmol) Na₂CO₃ and 12.5 g (84.2 mmol) sodium iodide was added and the mixture refluxed for 1.5 h. The cooled mixture was diluted with DMF, filtered, and the solid was washed with DCM. The filtrate was concentrated, diluted with DCM, and washed with 5% NaHCO₃ followed by washing with brine. The organic layer was dried over anhydrous Na₂SO₄, filtered, and concentrated. The residue was purified by silica column chromatography eluting with 10% EtOAc/hexane followed by 100% DCM to obtain 2.73 g of 13 (51% yield) as a yellow oil. LCMS expected 254.15, obtained 254.15.

### Example 3.3—Synthesis of 14

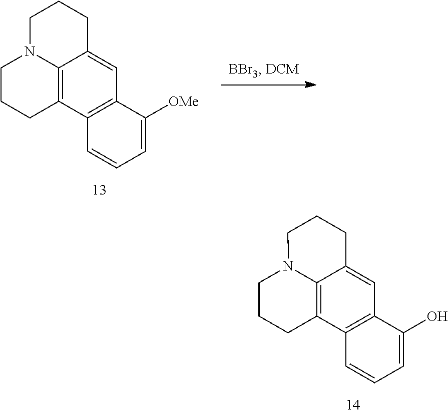

Route A: 13 (42 mg, 0.166 mmol) was dissolved in 4 mL anhydrous DCM in an oven-dried round-bottomed flask. Reaction mixture was cooled to −78° C. under Ar. BBr₃ (1 M in DCM, 0.2 mL, 0.2 mmol) was added dropwise and the mixture was stirred at −78° C. for 10 min, then allowed to warm to rt for 30 min. Mixture was cooled again to −78° C., then BBr₃ (1 M in DCM, 0.5 mL, 0.5 mmol) was added dropwise and the mixture was stirred at −78° C. for 10 min, then allowed to warm to rt for 1 h. Reaction mixture was cooled in an ice bath, quenched with 1 mL MeOH and stirred at rt for 1 h. sat. aq. NaHCO₃ (100 mL) was added, organics were extracted with DCM (3×100 mL), washed with brine (100 mL), dried over Na₂SO₄, filtered and concentrated in vacuo. Product was purified by silica column chromatography (75% DCM/hexanes-100% DCM) to obtain 12.7 mg of 14 (32% yield).

Alternative Route A′: 13 (2.73 g, 10.8 mmol) was mixed with 25 mL of 48% HBr and heated at 80° C. for 45 min followed by 120° C. for 20 min at which the mixture turned to a clear dark amber solution. The cooled solution was diluted with water and neutralized to pH 7. The resulting mixture was extracted 3 times with EtOAc, and the organic layer was washed with brine, dried over anhydrous Na₂SO₄, filtered, and concentrated. The residue was purified by silica column chromatography eluting with 0-5% MeOH/DCM to obtain 1.36 g of 14 (53% yield) as a greenish solid. LCMS expected 240.14, obtained 240.14.

### Example 3.4—Synthesis of 15

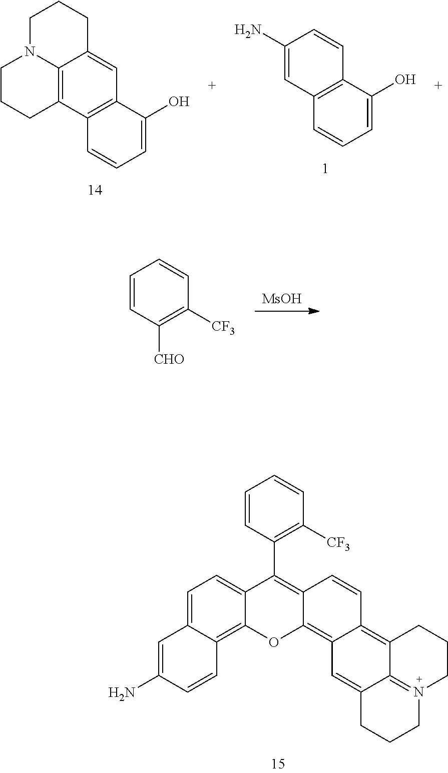

14 (10 mg, 0.0418 mmol), 6-amino-1-naphthol 1 (6.6 mg, 0.0418 mmol) and 2-(trifluoromethyl)benzaldehyde (5.5 μL, 0.0418 mmol) were dissolved in 0.5 mL methanesulfonic acid. The reaction mixture was heated to 150° C. for 2 h, then cooled to room temperature. The solution was transferred to a 50 mL centrifuge tube and the product was precipitated with diethyl ether (45 mL). The mixture was vortexed and centrifuged and the supernatant was decanted off. The solids were purified by column chromatography on C18 silica (50% MeOH/0.1% TFA in water-100% MeOH), followed by column chromatography on normal phase silica (2% MeOH/DCM-15% MeOH/DCM) to obtain 3.2 mg of 15 (21% yield).

### Example 3.5—Synthesis of 16

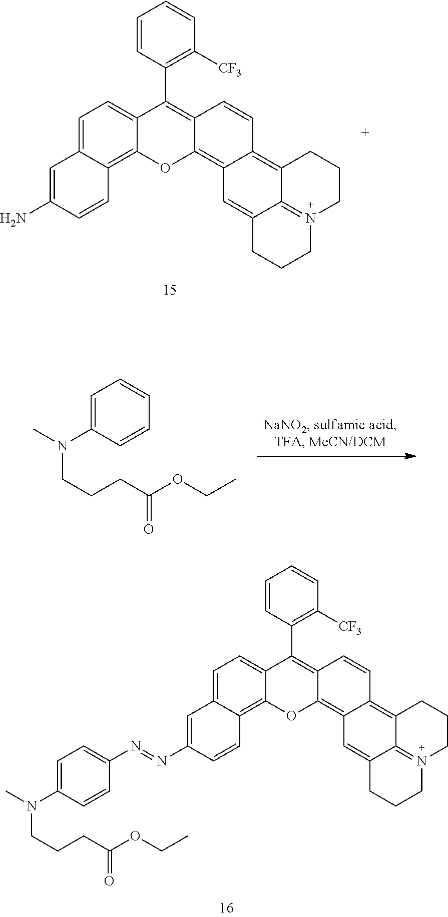

15 (3.2 mg, 0.00493 mmol) was dissolved in 1 mL 1:4 MeCN/DCM and cooled to 0° C. in an ice bath under argon atmosphere. To this solution was added sodium nitrite (1.4 mg, 0.0197 mmol) followed by trifluoroacetic acid (10 μL) and the reaction mixture was stirred for 10 min at 0° C. Sulfamic acid (2.0 mg, 0.0197 mmol) was added to the reaction mixture and the resulting solution was stirred for 5 min. After this time, a solution of ethyl 4-(methyl(phenyl)amino)butanoate (6.6 mg, 0.0296 mmol) dissolved in 0.2 mL MeCN was added dropwise to the reaction mixture. After 30 min, 50 mL water was added to the reaction mixture and the organics were extracted with DCM (3×50 mL), washed with brine (100 mL), dried over Na₂SO₄, filtered and concentrated. The solids were purified by column chromatography on silica (DCM-15% MeOH/DCM) to obtain 3.0 mg of 16 (69% yield).

### Example 3.6—Synthesis of 17

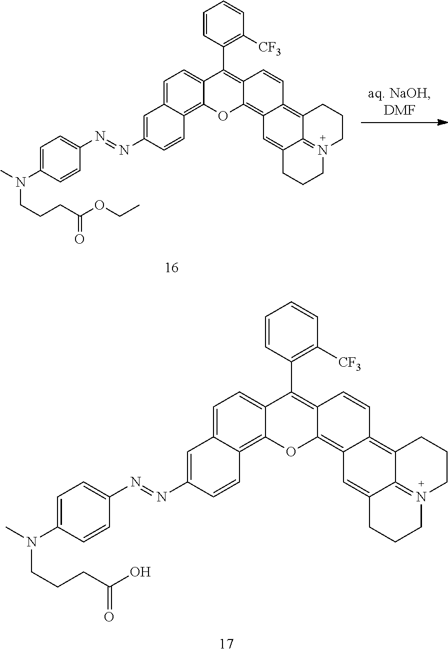

Compound 16 (3.0 mg, 0.00341 mmol) was dissolved in 1 mL DMF. To this solution was added 0.5 mL 1.0 M aq. NaOH and the resulting mixture was stirred at rt for 20 min. 0.6 mL 1.0 M aq. HCl was added to the solution, followed by 50 mL water. The organics were extracted with DCM (3×50 mL), washed with brine (100 mL), dried over Na₂SO₄, filtered and concentrated. The solids were purified by column chromatography on silica (DCM-22% MeOH/DCM) to obtain 1.4 mg of 17 (48% yield).

### Example 3.7—Synthesis of 18

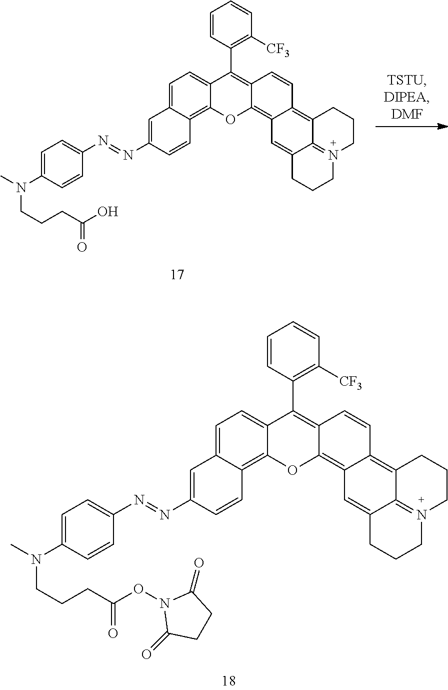

Compound 17 (1.4 mg, 0.00164 mmol) was dissolved in 1 mL anhydrous DMF. To this solution was added diisopropylethylamine (1.14 μL, 0.00657 mmol) and N,N,N′,N′-tetramethyl-O—(N-succinimidyl)uronium tetrafluoroborate (1.0 mg, 0.00328 mmol) and the resulting solution was stirred at rt for 30 min. DCM (50 mL) was added and the organics were washed with 10% aq. citric acid (3×50 mL) and brine (100 mL), dried over Na₂SO₄, filtered and concentrated. The solids were purified by column chromatography on silica (DCM-22% MeOH/DCM) to obtain 1.0 mg of 18 (64% yield).

### Example 4—Synthesis of Compound 20

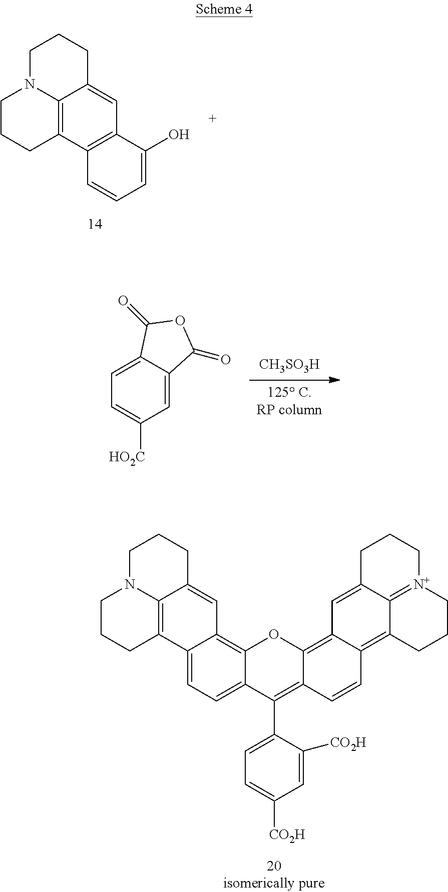

Compound 14 (14 mg, 0.058 mmol), trimellitic anhydride 19 (5.6 mg, 0.029 mmol), and methanesulfonic acid (1.5 mL) were heated at 125° C. for 3h. Upon cooling, the reaction solution was poured into 40 mL of water to yield a blue precipitate. The mixture was centrifuged, and the pellet was dried under vacuum. The solid was column chromatographed on C18 silica eluting with 40-90% MeOH/TEAA to yield two isomeric products. The second eluting product was desalted on a small pad of C18 silica, concentrated, and dried to 6 mg of 20 as a purple colored solid. LCMS expected 635.25, obtained 635.25. Absorbance max 690 nm.

### Example 5—Synthesis of Compound 26 Attached to a Solid Support (Construct 29)

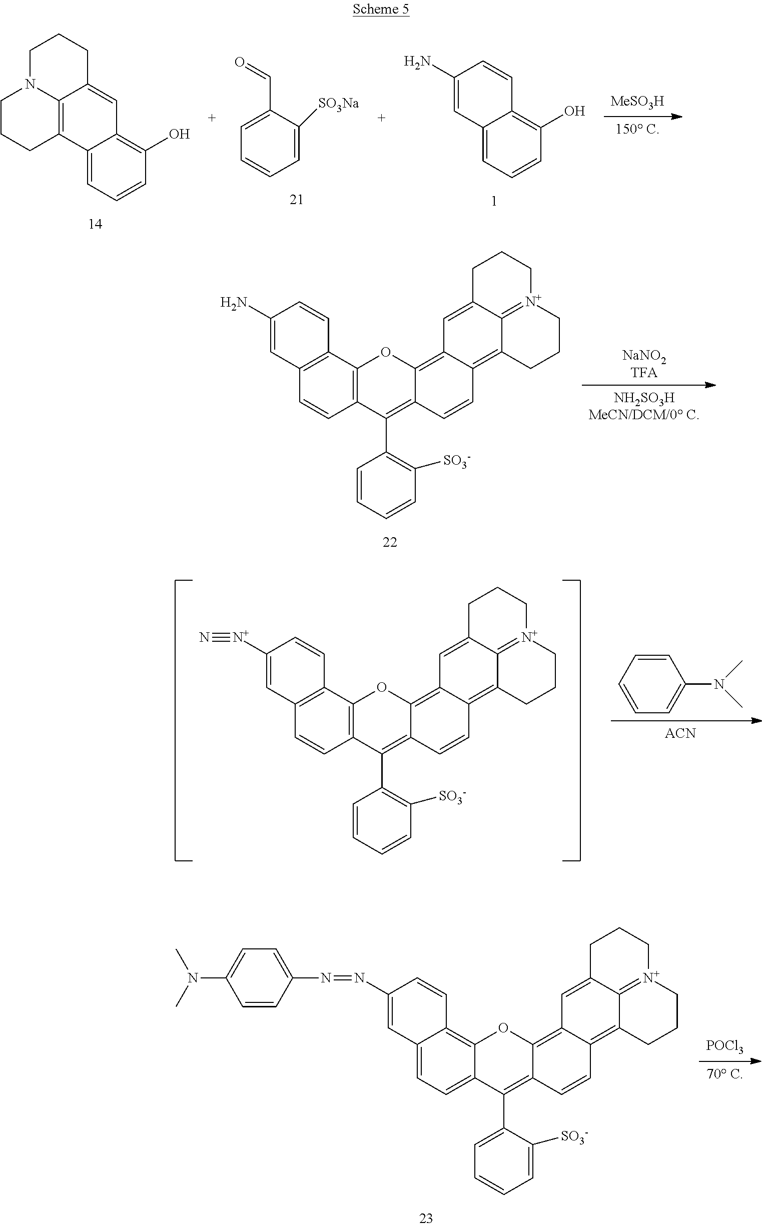

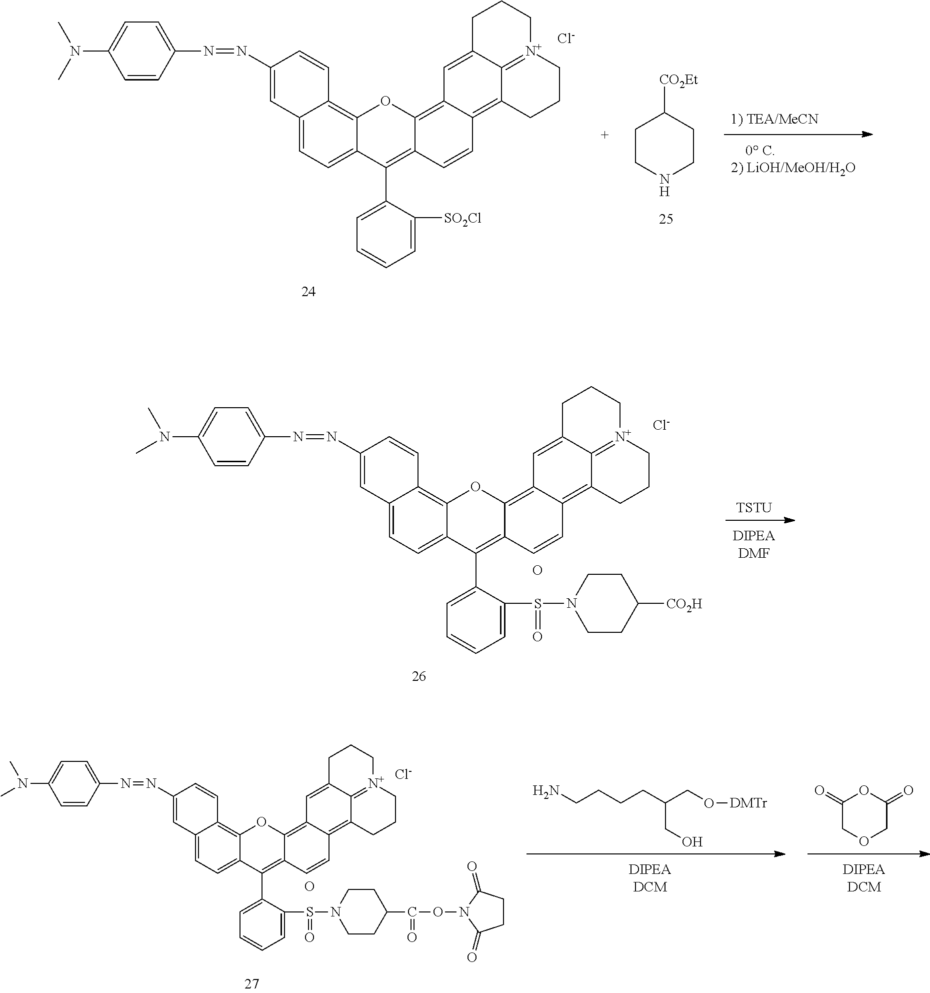

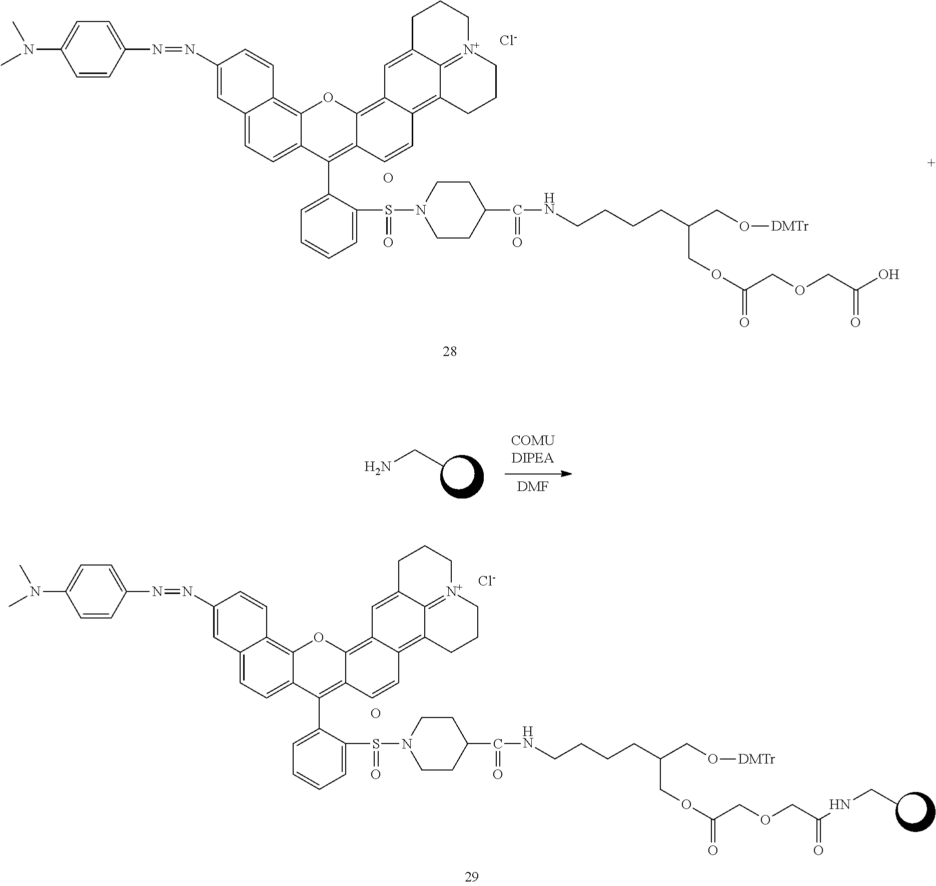

### Example 5.1—Synthesis of 22

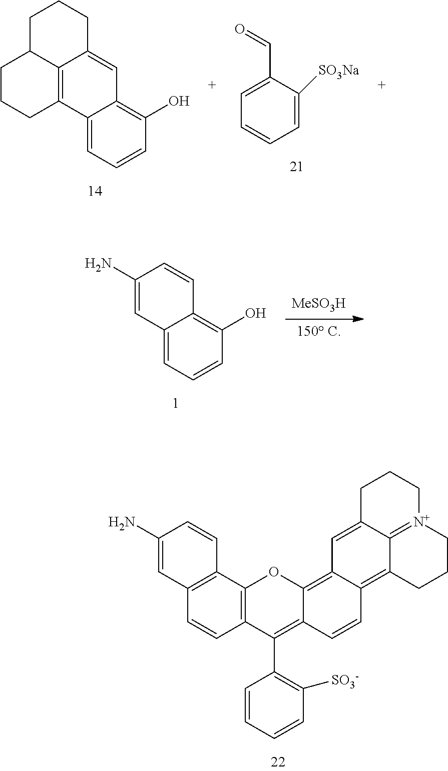

6-amino-1-naphthol (1, 133 mg, 0.836 mmol), 2-sulfobenzaldehyde (21, 174 mg, 0.836 mmol), and 14 (200 mg, 0.836 mmol) were mixed in 10 mL of methanesulfonic acid and heated at 150° C. for 2.5 h. The reaction solution was mixed with 135 mL ether and the precipitate was collected by centrifugation. The solid pellet was purified on silica eluting with 5-15% MeOH/DCM. This was further purified on C18 silica eluting with 70% MeOH/0.1% aq TFA, water, MeOH, and MeOH/DCM to yield 103 mg (23% yield) of 22 as a dark blue solid. LCMS expected 547.169, obtained 547.167.

### Example 5.2—Synthesis of 23

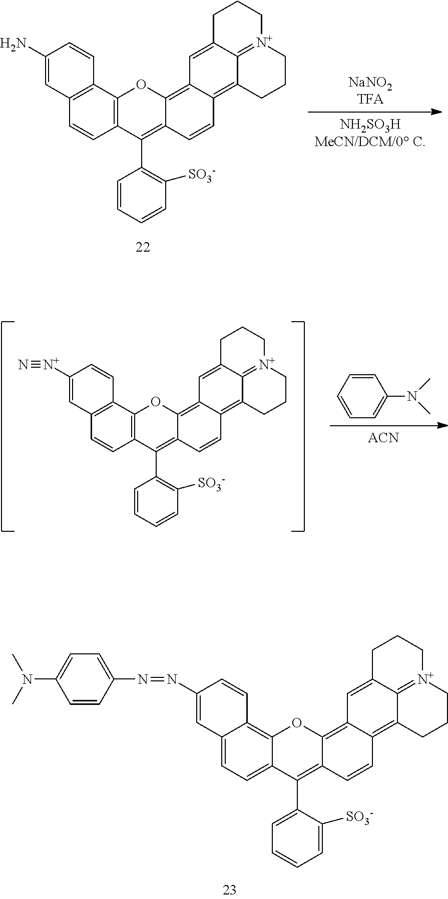

Compound 22 (84 mg, 0.16 mmol) was dissolved in a solution of MeCN (3.5 mL), DCM (12 mL), and TFA (0.17 mL) and cooled to 0° C. in an ice bath with stirring to give a dark blue solution. Sodium nitrite (17 mg, 0.24 mmol) was added and stirred for 10 min. Then, sulfamic acid (23 mg, 0.24 mmol) was added to the green solution and stirred for 10 min. Dimethylaniline was dissolved in 2.5 mL of MeCN and added dropwise to the cold diazonium solution. The resulting purple solution was further stirred at 0° C. for 1 h then warmed to room temperature. The solvent was evaporated, and the resulting solid was purified by column chromatography on silica eluting with 100% DCM then 0.5% TFA/10-15% MeOH/DCM. The concentrated product was then desalted on C18 silica washing with 50% MeOH/H₂O, followed by eluting with MeOH and 10% MeOH/DCM to obtain 62 mg of 23 (57% yield) as a dark purple-blue solid. LCMS expected 679.237, obtained 679.234. Absorbance max 700 nm (range 600-800 nm), no fluorescence.

### Example 5.3—Synthesis of 24

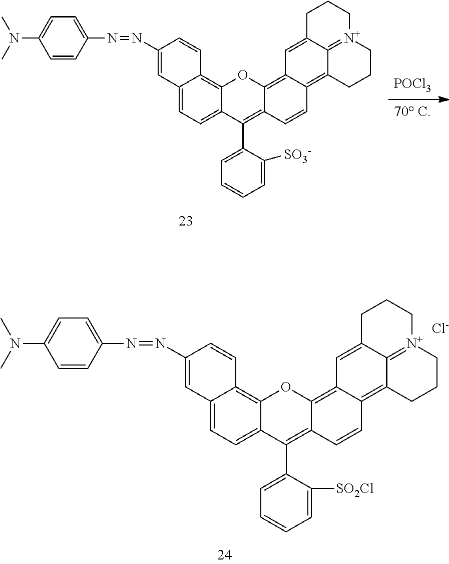

Compound 23 (4.6 mg, 0.0068 mmol) was mixed with 1 mL of phosphorous oxychloride and heated at 70° C. under N₂ for 2 h. The POCl₃ was removed by evaporation the resulting solid further dried under vacuum. The resulting purple solid, 24, was used as is. LCMS expected 697.203, obtained 697.201.

### Example 5.4—Synthesis of 26

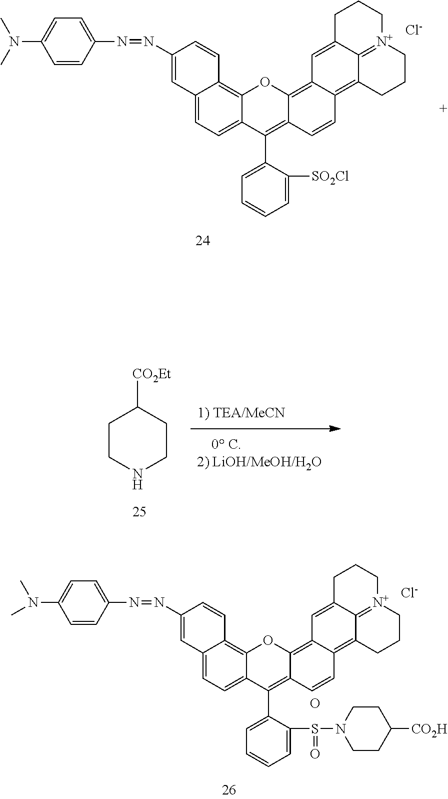 [SMILES: CN(C)c1ccc(N=Nc2ccc3c4c(ccc3c2)C(c2ccccc2S(=O)(=O)Cl)=c2ccc3c5c6c(cc3c2O4)CCC[N+]=6CCC5)cc1 ; [Cl-] ; CCOC(=O)C1CCNCC1 ; CN(C)c1ccc(N=Nc2ccc3c4c(ccc3c2)C(c2ccccc2S(=O)N2CCC(C(=O)O)CC2)=c2ccc3c5c6c(cc3c2O4)CCC[N+]=6CCC5)cc1 ; O ; [Cl-]]

Ethyl 4-piperidine carboxylate (25, 189 mg, 1.2 mmol), triethylamine (167 uL, 1.2 mmol), and dry acetonitrile were cooled in an ice bath. Compound 24 (85 mg, 0.12 mmol), dissolved in 20 mL dry acetonitrile, was then added dropwise. The reaction solution was concentrated and purified by column chromatography on silica gel eluting with 0-10% MeOH/DCM to give 94 mg (91% yield) of the ethyl ester intermediate as a dark green solid. The ethyl ester was cleaved by dissolving in 10 mL of dry DMF and adding 4 mL of 1 M aqueous sodium hydroxide. This was stirred for 1 h at rt. The reaction solution was diluted with water and extracted 4 times with DCM. The organic layer was dried over anhydrous sodium sulfate, filtered, and concentrated to dryness to give 58 mg (65% yield) of 26 as a dark green solid. LCMS expected 790.306, obtained 790.306.

### Example 5.5—Coupling of Compound 26 to Solid Support

### Synthesis of 27:

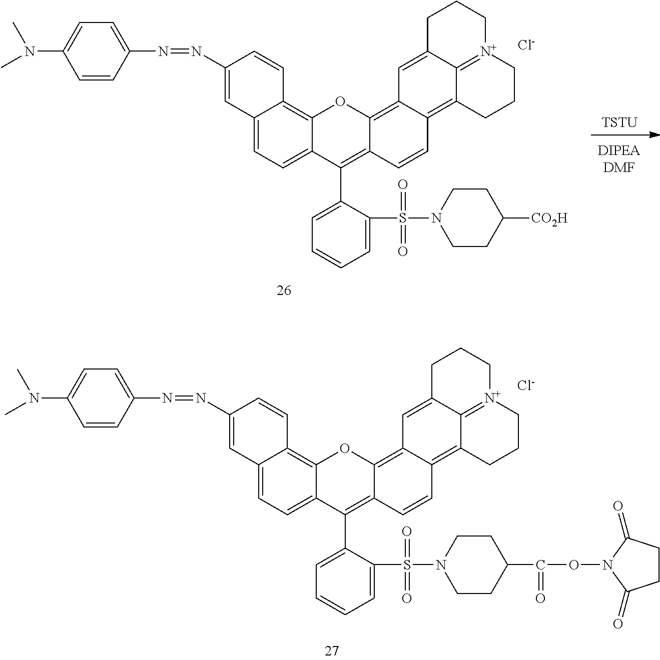 [SMILES: CN(C)c1ccc(N=Nc2ccc3c4c(ccc3c2)C(c2ccccc2S(=O)(=O)N2CCC(C(=O)O)CC2)=c2ccc3c5c6c(cc3c2O4)CCC[N+]=6CCC5)cc1 ; [Cl-] ; "TSTU" ; CN(C)c1ccc(N=Nc2ccc3c4c(ccc3c2)C(c2ccccc2S(=O)(=O)N2CCC(C(=O)ON3C(=O)CCC3=O)CC2)=c2ccc3c5c6c(cc3c2O4)CCC[N+]=6CCC5)cc1 ; [Cl-]]

Compound 26 (57 mg, 0.069 mmol) was dissolved in 10 mL dry DMF. N,N,N′,N′-tetramethyl-O—(N-succinimidyl)uronium tetrafluoroborate (42 mg, 0.14 mmol) and diisopropylethylamine (24 uL, 0.14 mmol) were added and the solution stirred at rt for 1 h. The reaction solution was concentrated to remove most of the DMF, and the resulting residue was dissolved in DCM and washed once each with 1 M HCl, water, and brine. The organic layer was dried over anhydrous sodium sulfate, filtered, and concentrated to give 49 mg (77% yield) of a dark green solid. LCMS expected 887.322, obtained 887.323.

### Synthesis of Construct 28:

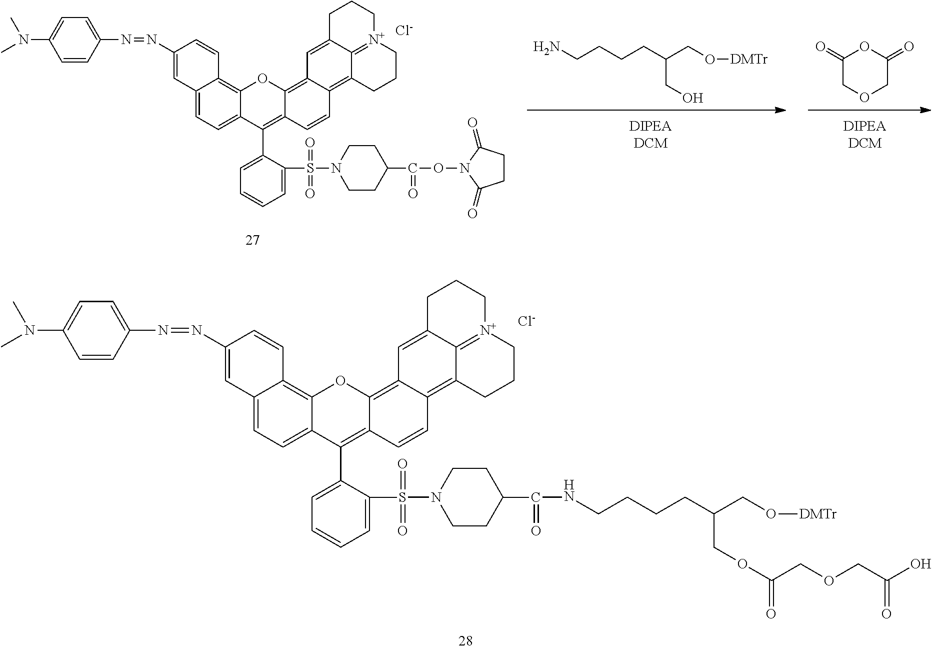

Compound 27 (50 mg, 54 mmol) was dissolved in 10 mL of dry DCM. In an addition funnel were mixed ODMT-aminobutyl-1,3-propanediol (27 mg, 60 mmol, 110 mM in DCM) and diisopropylamine (14 uL, 81 mmol) which was added to 27 dropwise with stirring. After stirring at rt for 45 min, the reaction solution was diluted with DCM, and washed one time each with 1% citric acid, water, and brine. This was dried over anhydrous sodium sulfate, filtered, and concentrated to give 58 mg (85% yield) of crude DMT protected linker intermediate. This was dissolved in 10 mL dry DCM and diisopropylamine (20 uL, 115 mmol) and diglycolic anhydride (17 mg, 146 mmol) were added. This was stirred at rt for 3.5 h. The reaction solution was concentrated and purified by column chromatography on silica gel eluting with 0-20% MeOH/DCM/1% TEA to give 31 mg (49% yield) of 28 as a green solid. LCMS (positive ion, negative buffer) expected 1337.563, obtained 1337.560.

### Synthesis of Construct 29:

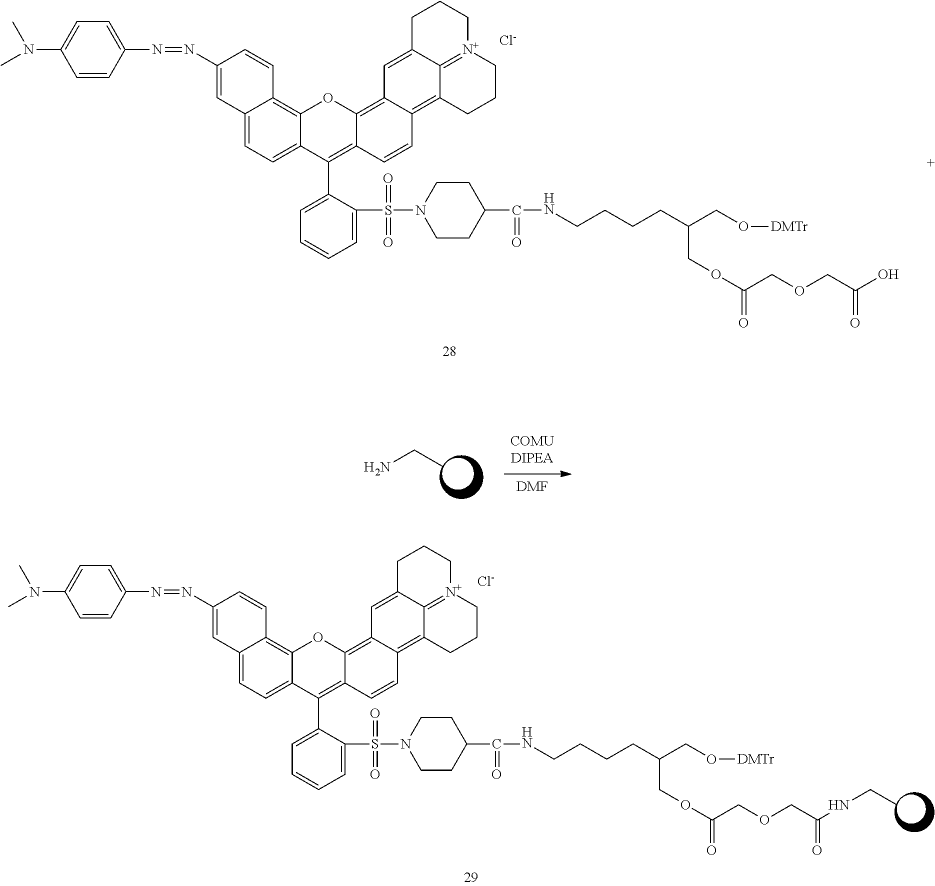

The dye glycolic linker isomers 28 (12 mg, 8.7 μmol) was dissolved in 3.5 mL dimethylformamide. AM-polystyrene 33 μmol/g (264 mg, 6.7 μmol) was added to the flask followed by 7.7 μL diisopropylethylamine and then 2-cyano-2-(hydroxyamino)acetate (oxyma) (COMU 3.7 mg, 8.7 μmol). The reaction was placed on a shaker. After 3 h the solid was filtered and washed 3 times with 4 mL DMF, then 3 times with 4 mL acetonitrile, and then 3 times with 4 mL dichloromethane. The solid was placed under vacuum to dry overnight. 4.3 mg of the solid was added to a volumetric flask that was then filled with a toluenesulfonic acid in acetonitrile solution. The absorbance was measured at 498 nm and the loading amount of 28 onto the AM-polystyrene support was determined to be 20 μmol/g. The solid support was added to a flask followed by capping reagents N-methylimidazole/THF (25 mL) and acetic anhydride/pyridine/THF (2 mL). The flask was placed on a shaker for 1 h. The solid was then filtered and washed 3 times with 4 mL THF, followed by 3 times with 4 mL acetonitrile and then 3 times with 4 mL dichloromethane. The solid support was dried overnight under high vacuum to provide structure 29.

### Example 6—Synthesis of Compound 35

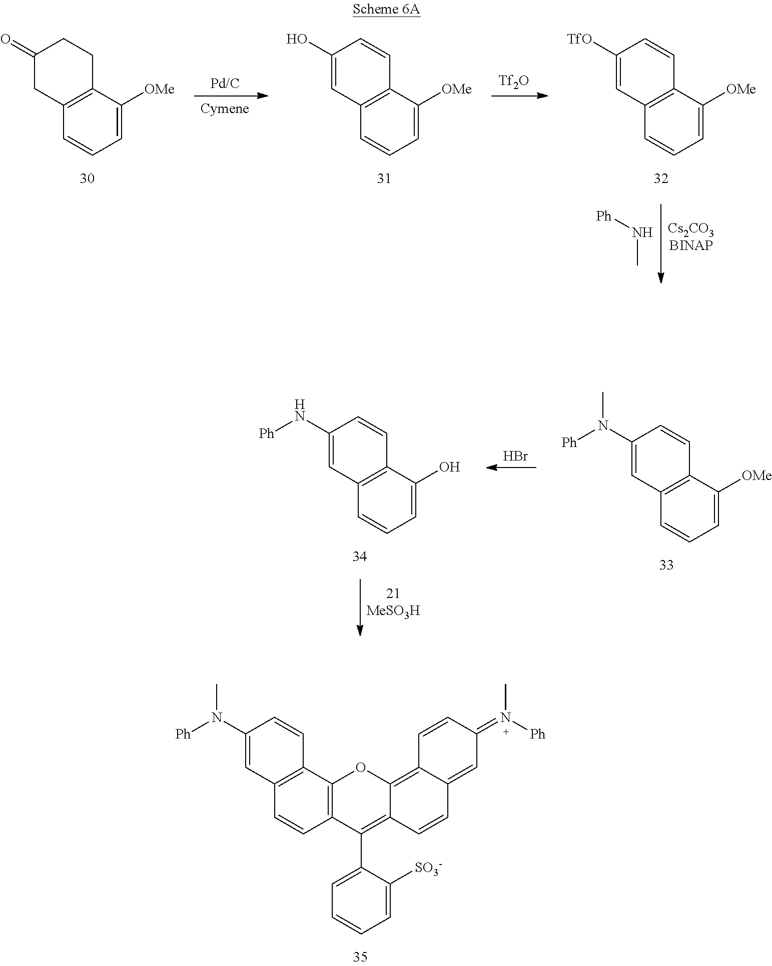

### Example 6.1—Synthesis of 31

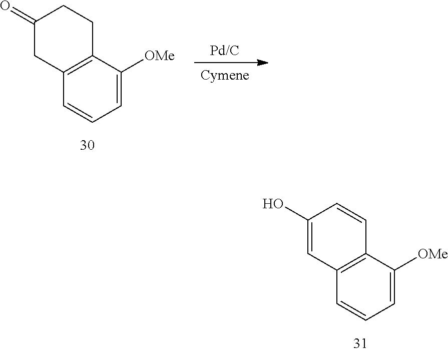 [SMILES: COc1cccc2c1CCC(=O)C2 ; "Cymene" ; "Pd/C" ; COc1cccc2cc(O)ccc12]

5-methoxy-2-tetralone 30 (2.5 g, 14.1 mmol) and 10% Pd/C (750 mg) were refluxed in 20 mL of cymene for 48 h. The mixture was cooled to rt, diluted with dichloromethane, and extracted with 2 N NaOH (pH˜12). The aqueous layer was then acidified with 6 M HCl to pH 2 and the precipitated product was extract with dichloromethane, washed with brine, dried with sodium sulfate, filtered, and concentrated by rotovap. The product 5-methoxynapthalene-2-ol 31 was purified by silica column (100% dichloromethane).

### Example 6.2—Synthesis of 32

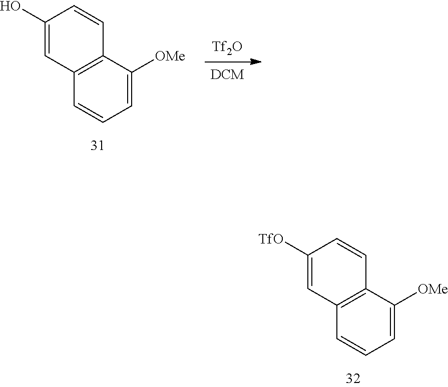

Under nitrogen, 5-methoxynapthalene-2-ol 31 (650 mg, 3.7 mmol) was dissolved in 3 mL DCM and stirred in an ice bath at 0° C. Triethylamine (1.2 mL) was then added and stirring continued for 5 min. Triflic acid (1.2 mL) was then added dropwise and the reaction was allowed to warm to rt and stirred for 5 h. The mixture was decomposed with ice and extracted with DCM, dried with sodium sulfate, filtered, and concentrated by rotovap. The product 5-methoxynaphthalen-2-yl trifluoromethanesulfonate 32 was purified by silica column chromatography (3% EtOAc/Hexanes).

### Example 6.3—Synthesis of 33

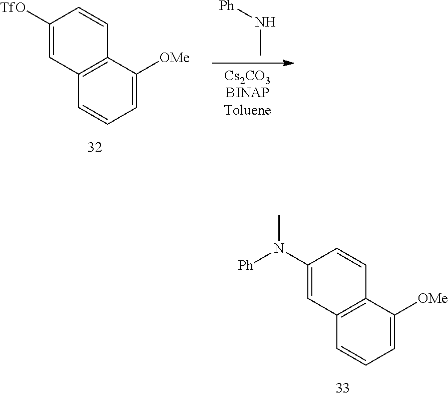

Under nitrogen, 5-methoxynaphthalen-2-yl trifluoromethanesulfonate 32 (782 mg, 2.5 mmol), BINAP (477 mg, 0.76 mmol), palladium acetate (115 mg, 0.51 mmol), and cesium carbonate (2.329 g, 7.15 mmol) were added to a round bottom flask and the flask was sealed. N-methylaniline (650 μL, 6.1 mmol) was mixed with 8 mL toluene and added to the flask and the reaction was stirred at 100° C. overnight. Methanol and silica gel were added to the reaction mixture and the solvent was removed by rotovap. The product 5-methoxy-N-methyl-N-phenylnaphthalen-2-amine 33 was purified by column chromatography (1.5% EtOAc/Hexanes). Yield: 82%. LCMS expected 264.1383, obtained 264.1377.

### Example 6.4—Synthesis of 34

### Route A to 34:

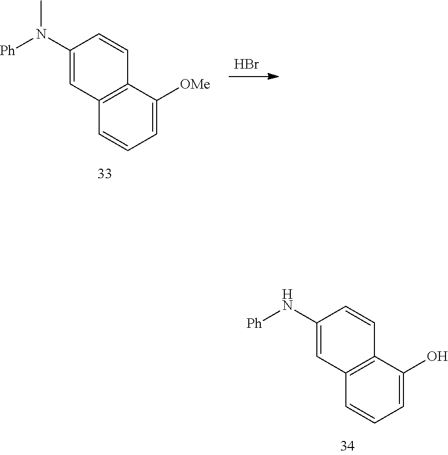 [SMILES: COc1cccc2cc(N(C)c3ccccc3)ccc12 ; Br ; Oc1cccc2cc(Nc3ccccc3)ccc12]

Under nitrogen, 5-methoxy-N-methyl-N-phenylnaphthalen-2-amine 33 (550 mg, 2.09 mmol) was heated in 6 mL hydrobromic acid at 110° C. for 3 h. The solution was neutralized with 6 M NaOH and extracted with DCM, dried with sodium sulfate, filtered, and concentrated by rotovap. The product 6-(methyl(phenyl)amino)naphthalen-1-ol 34 was purified by column chromatography (6% EtOAc/Hexanes). Yield 73%. LCMS expected 150.1226, obtained 150.1222.

### Example 6.5—Synthesis of 35

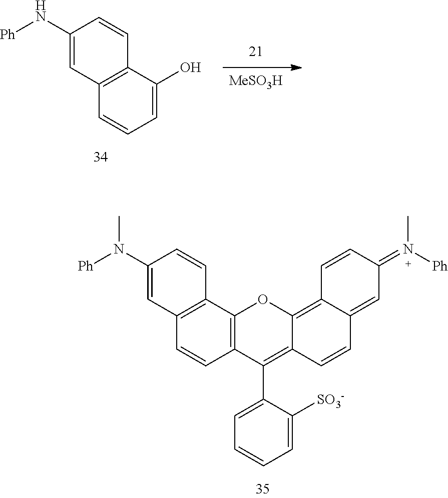

Under nitrogen, 6-(methyl(phenyl)amino)naphthalen-1-ol 34 (0.379 mg, 1.52 mmol) and sodium 2-formylbenzenesulfonate 21 (0.158 mg, 0.76 mmol) were dissolved in 4 mL methanesulfonic acid and heated at 150° C. for 5 h. The solution was cooled to room temperature and precipitated in ether, centrifuged, and the ether was decanted from the pellet which was dried overnight. The rhodamine 35 was purified by column chromatography (5% MeOH/DCM) LCMS expected 647.1999, obtained 647.1999.

### Route B to 34:

### Scheme 6B: Alternative Method of Synthesis of Compound 34 for Synthesis of 35

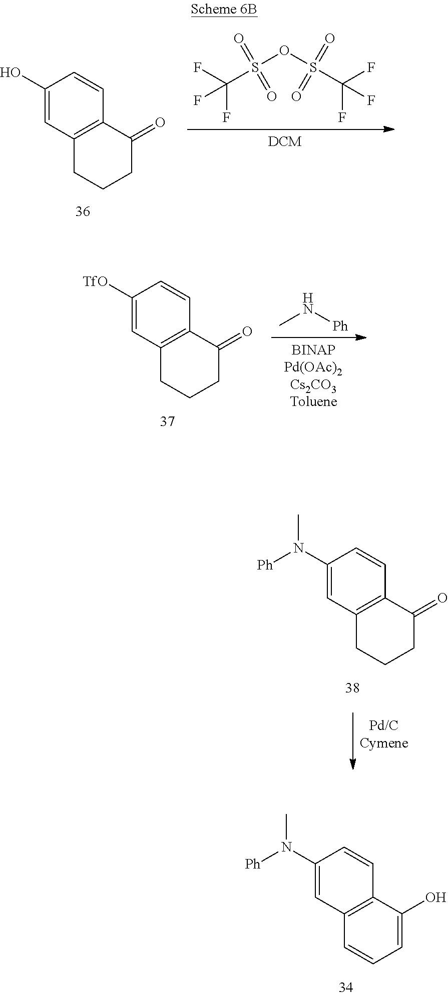

### Example 6.6—Synthesis of 37

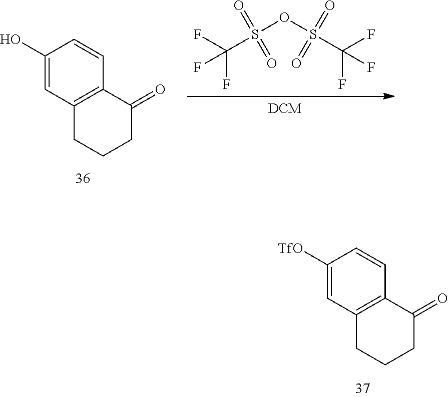

Under nitrogen, 6-hydroxy-3,4-dihydronaphthalen-1(2H)-one 36 was dissolved in DCM and stirred in an ice bath at 0° C. Triethylamine was then added and the reaction mixture was stirred for 5 min. Triflic acid was then added dropwise and the reaction was allowed to warm to rt and stir for 5 h. The mixture was decomposed with ice and extracted with DCM, dried with sodium sulfate, filtered, and concentrated by rotory evaporation.

### Example 6.7—Synthesis of 38

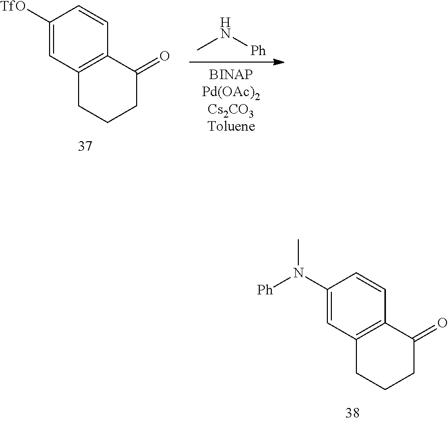

Under nitrogen, 5-oxo-5,6,7,8-tetrahydronaphthalen-2-yl 37, BINAP, palladium acetate, and cesium carbonate were added to a round bottom flask and the flask was sealed. N-methylaniline was mixed with toluene and added to the flask and the reaction was stirred at 100° C. overnight.

### Example 6.8—Synthesis of 34

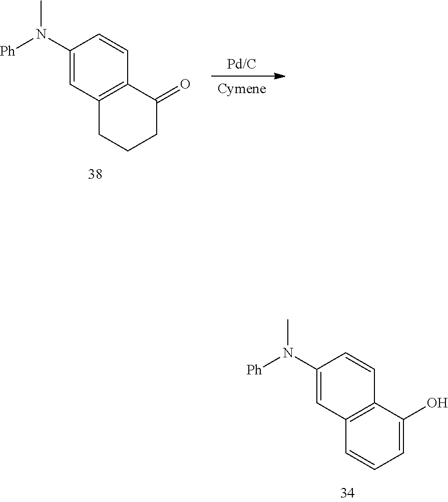 [SMILES: CN(c1ccccc1)c1ccc2c(c1)CCCC2=O ; "Cymene" ; "Pd/C" ; CN(c1ccccc1)c1ccc2c(O)cccc2c1]

6-(methyl(phenyl)amino)-3,4-dihydronaphthalen-1(2H)-one 38 and 10% Pd/C were refluxed in cymene for 48 h. The mixture was cooled to rt diluted with dichloromethane and extracted with 2 N NaOH (pH˜12). The aqueous layer was then acidified with 6 M HCl to pH 6 and the precipitated product was extracted with dichloromethane, washed with brine, dried with sodium sulfate, filtered, and concentrated by rotory evaporation.

### Example 6.9—Coupling of Compound 25 to Solid Support

Compound 25 can be coupled to a solid support according to the reactions shown in Scheme 6C.

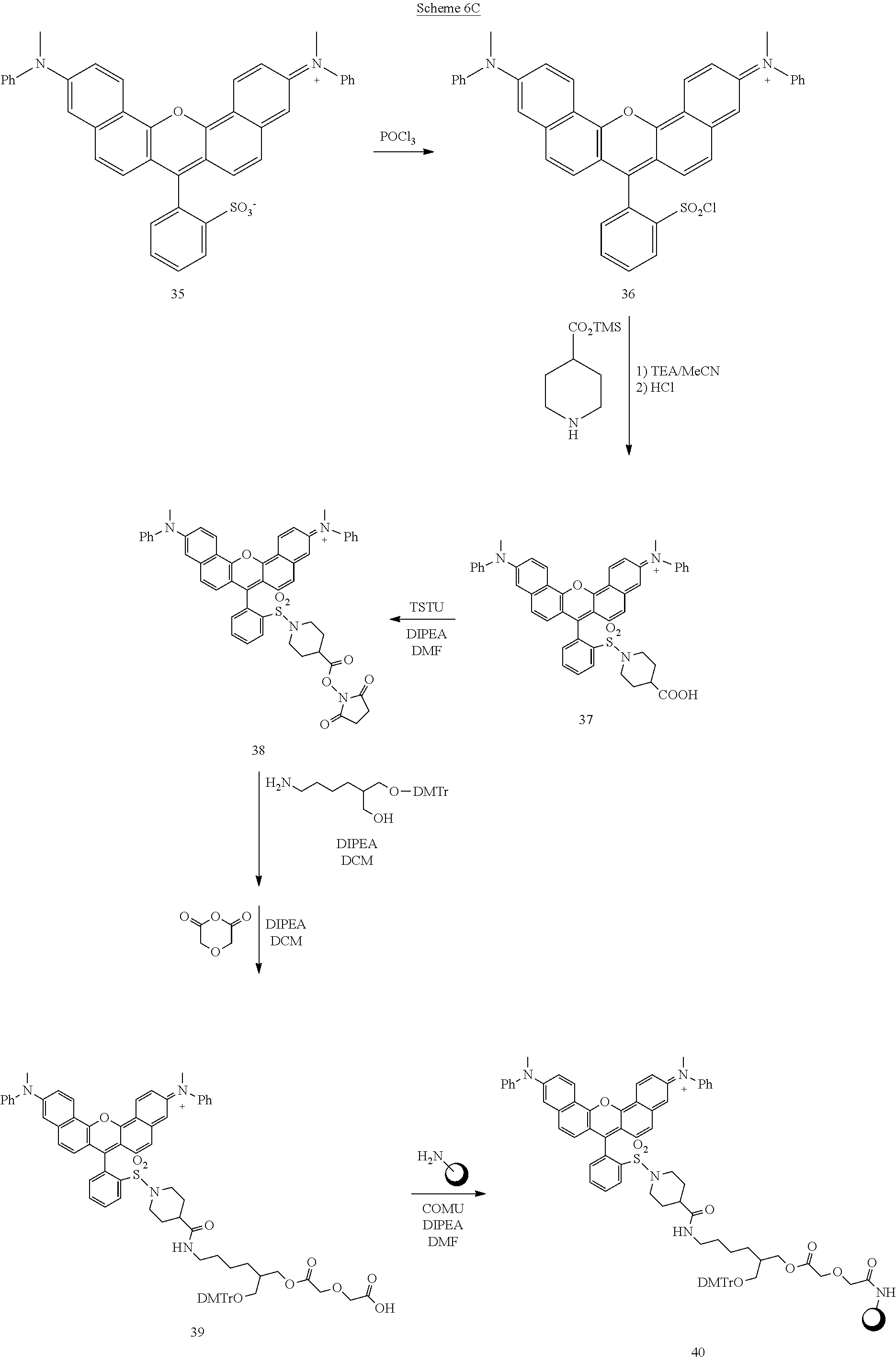

The exemplary synthetic procedures set forth herein may be easily generalized to any of the quenchers described herein, including the compounds set forth in Examples 7-11.

### Example 7—Synthesis of Compound 44

### Scheme 7A—Route A to 44:

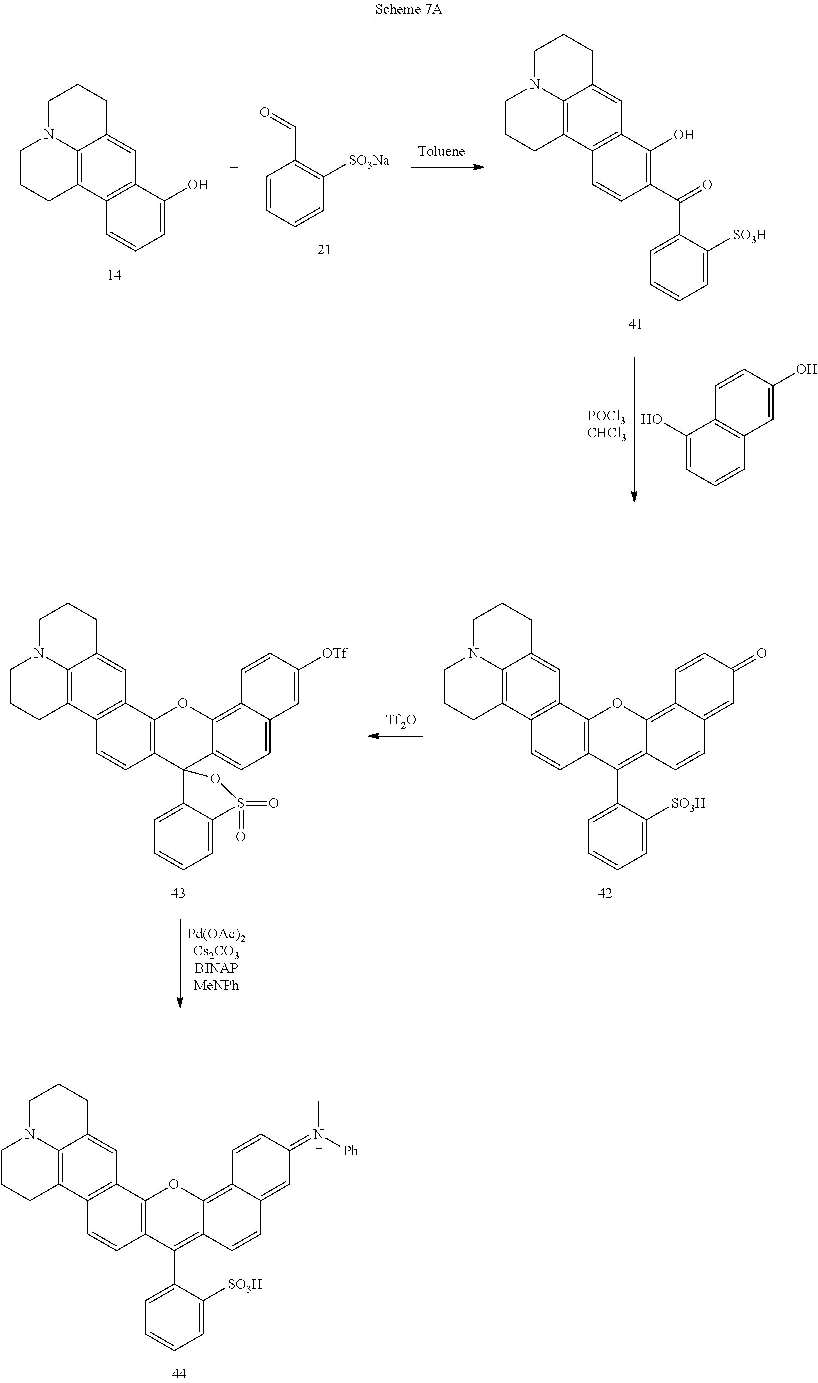

### Scheme 7B—Route B: Alternative Synthesis for 44:

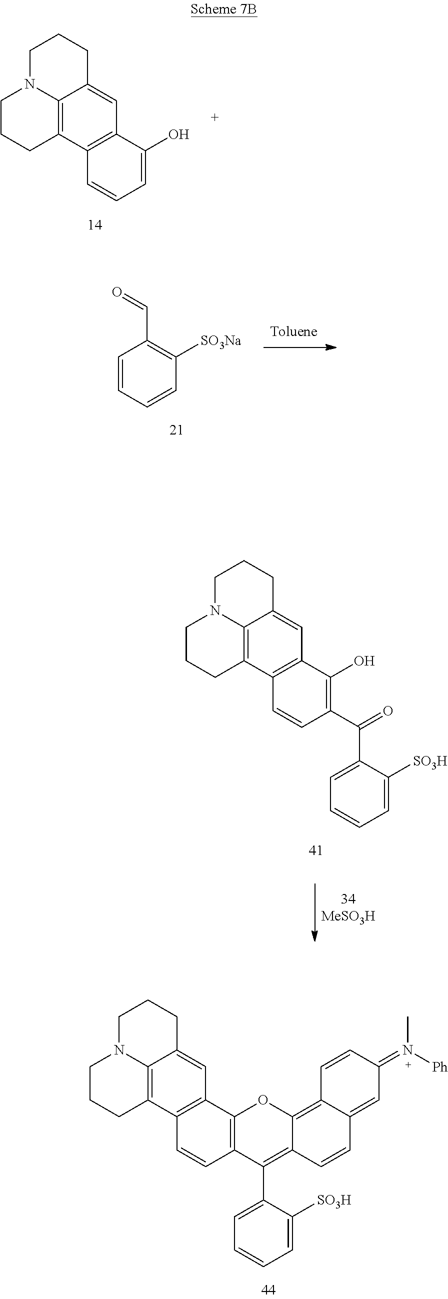

### Scheme 7C—Route C: Alternative Synthesis for 44:

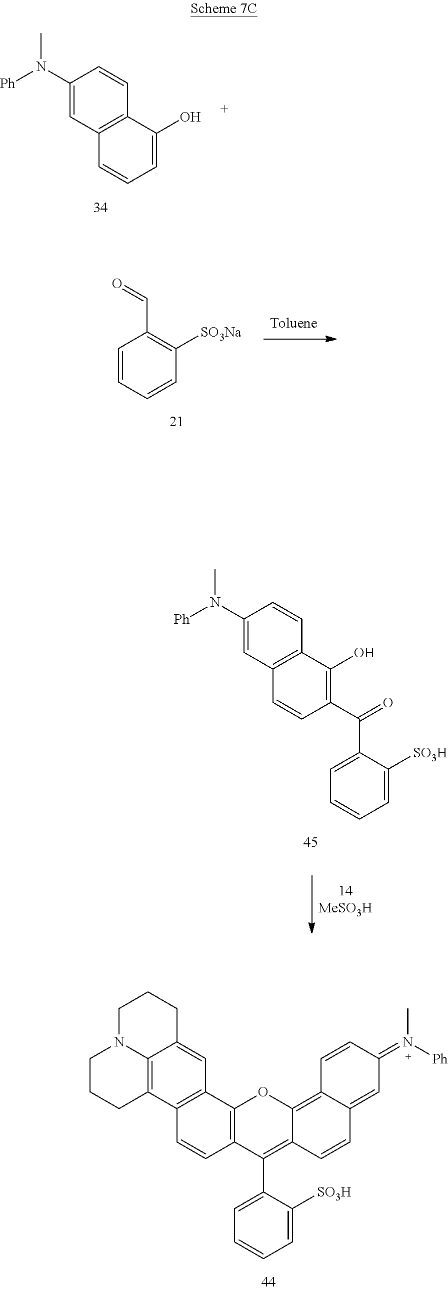

### Scheme 7D—Route D: Alternative Synthesis for 44:

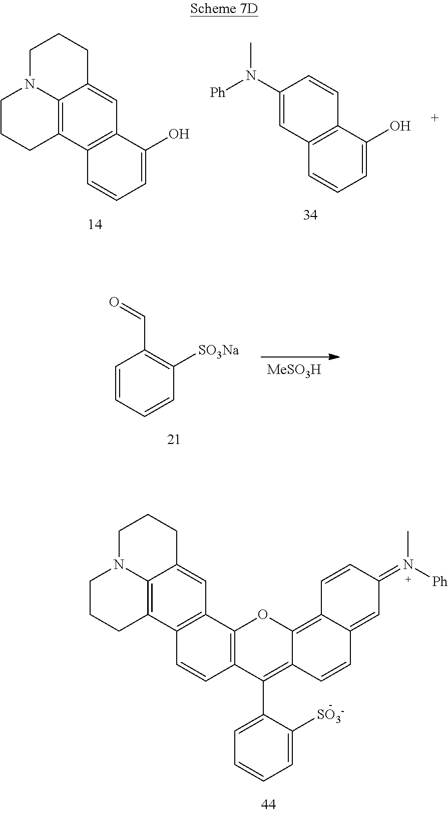

Compounds 14 and 34 are synthesized as previously described.

Under nitrogen, 6-(methyl(phenyl)amino)naphthalen-1-ol 34, 2,3,6,7-tetrahydro-1H,5H-benzo[f]pyrido[3,2,1-ij]quinolin-9-ol 14, and sodium 2-formylbenzenesulfonate 21 are dissolved methanesulfonic acid and heated at 150° C. for 5 h. The solution is cooled to room temperature and precipitated in ether, centrifuged, and the ether is decanted from the pellet (44) which is dried overnight.

### Example 8—Synthesis of Compounds 46, 47, 48

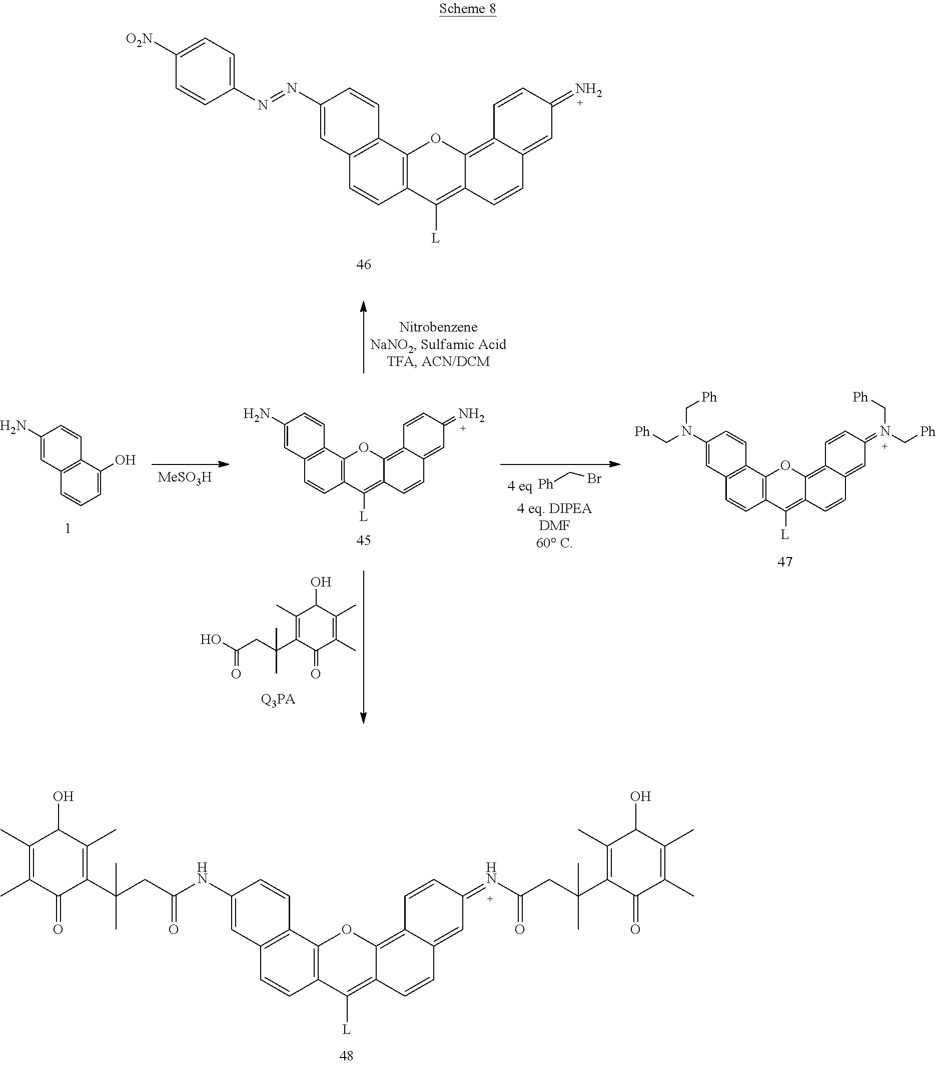

### Example 9—Synthesis of Compounds 51 and 52

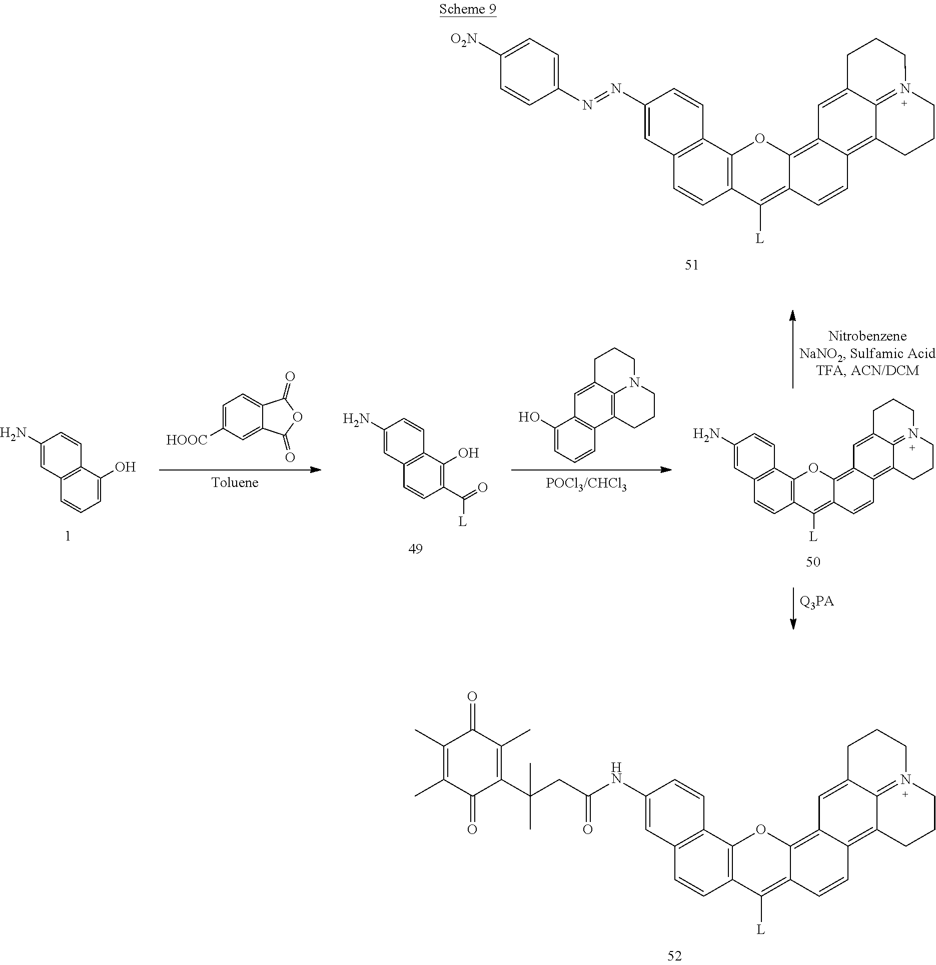

### Example 10—Synthesis of Compounds 56 57, and 59

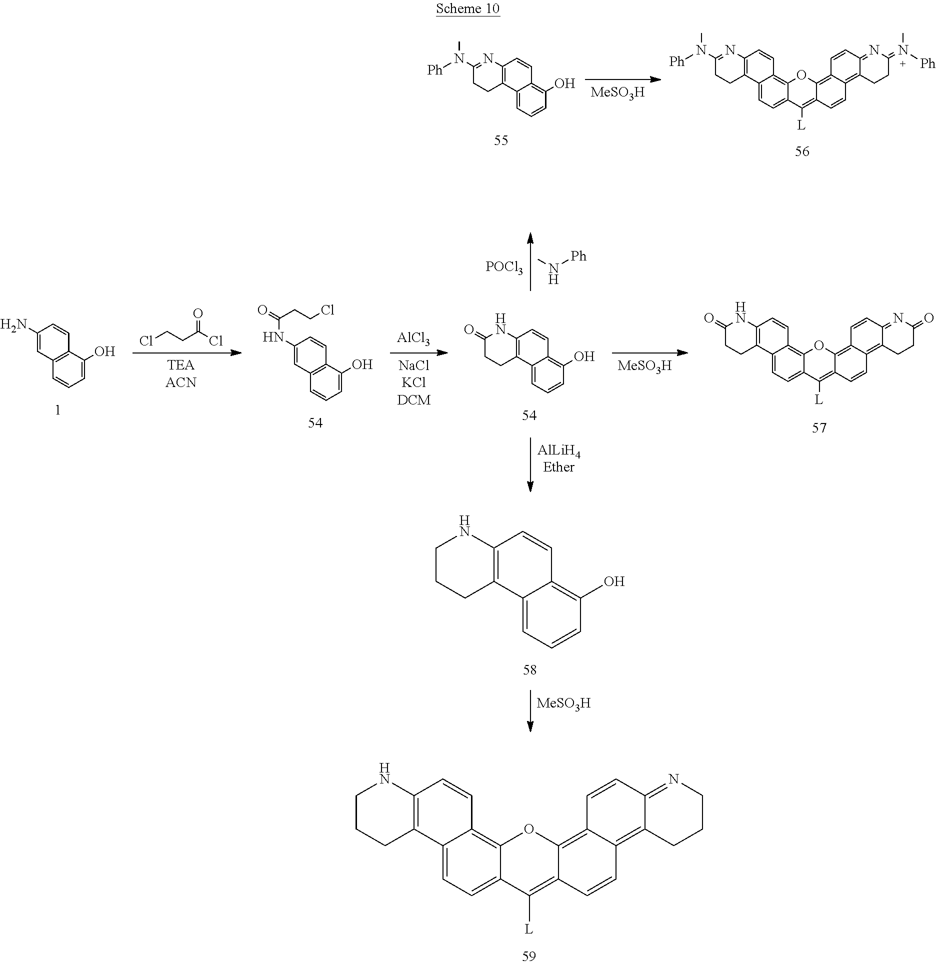

### Example 11—Synthesis of Compound 65

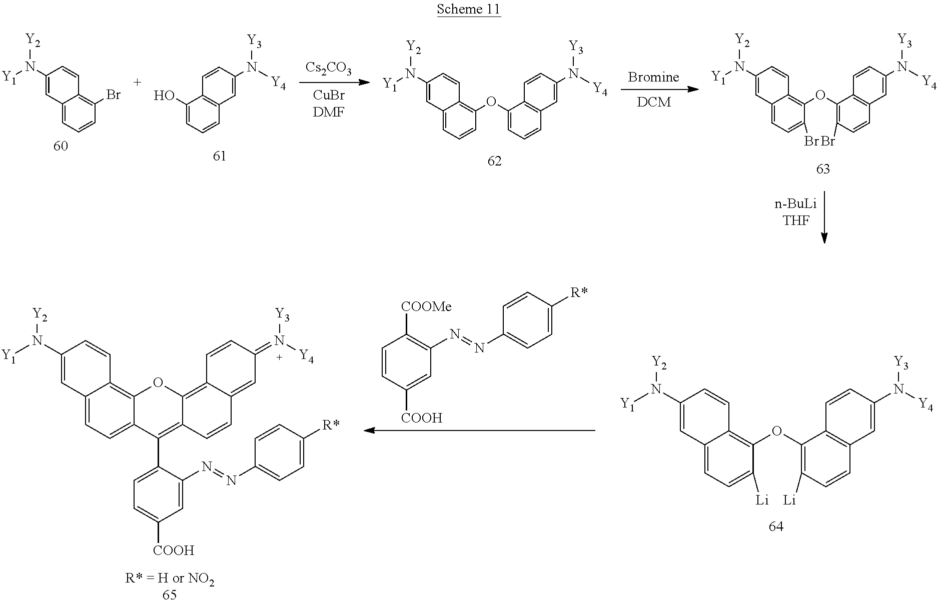

### Example 12—Synthesis of Compound 66

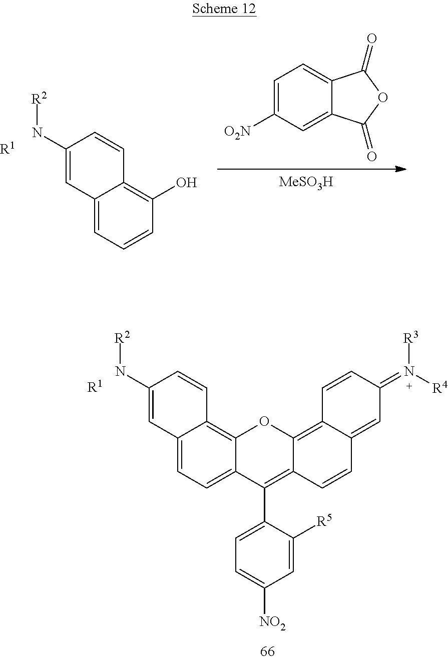

### Example 13—Synthesis of Compound 67

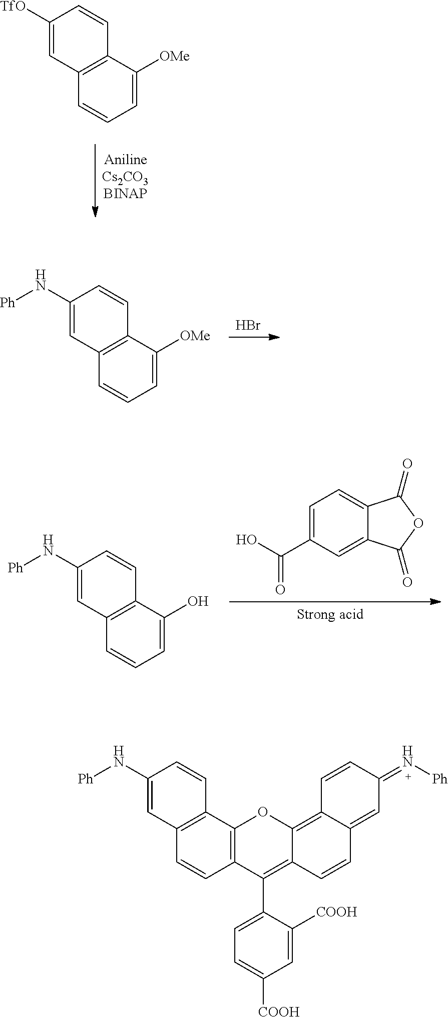

### Example 13—Synthesis of Compounds 68 and 69

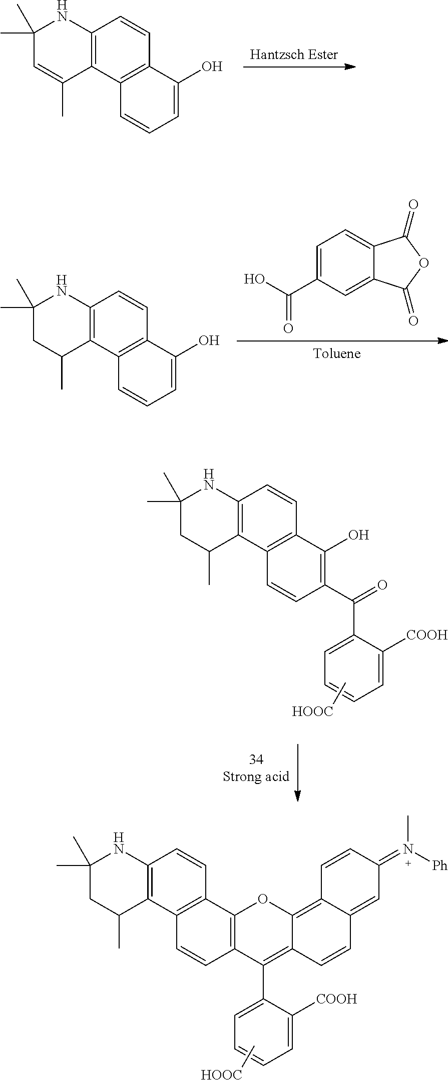

### Example 13—Synthesis of Compounds 70-72

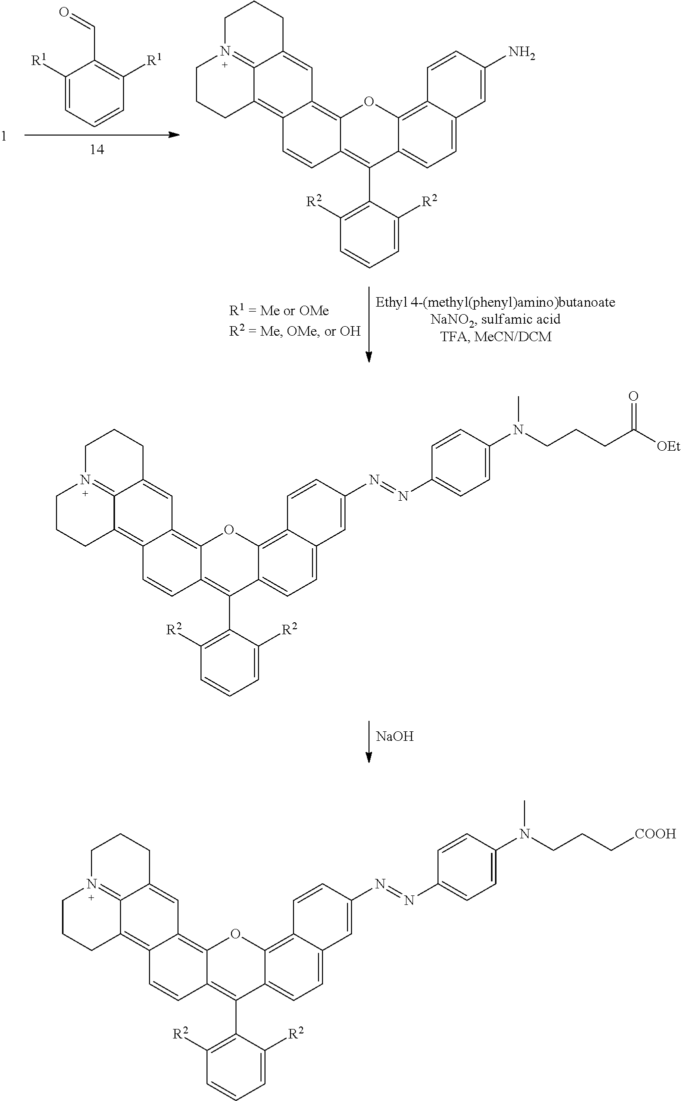

### Example 16—Synthesis of Compound 73

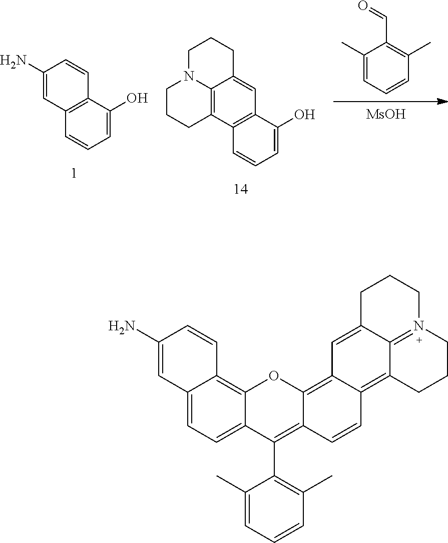

### Example 17—Synthesis of Compounds 74 and 75

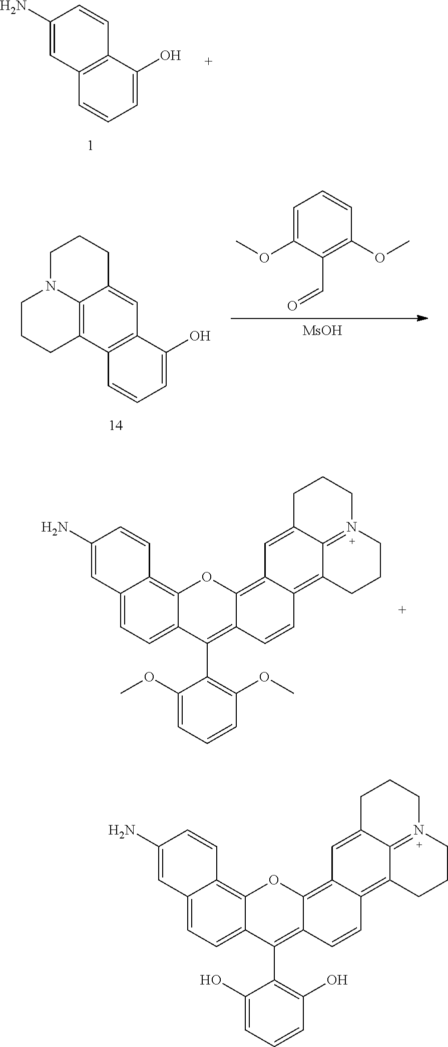

### Comparative Testing Example—Fluorescence Quenching Experiment

The efficacy of an oligonucleotide probe linked to quencher 3 for three different reporter dyes was tested: Reporter Dye 1 (excitation maxima at 650 nm; emission maxima at 671 nm), Reporter Dye 2 (excitation maxima at 682 nm; emission maxima at 697 nm), and Reporter Dye 3 (excitation maxima at 699 nm; emission maxima at 722 nm), each having emission maxima in the far-red/near-IR region of the electromagnetic spectrum. The oligonucleotide probe was prepared by automated oligonucleotide synthesis from Construct 8, followed by deprotection and cleavage from the solid support using methods well known to those in the art.

### General Snake Venom Digest Procedure

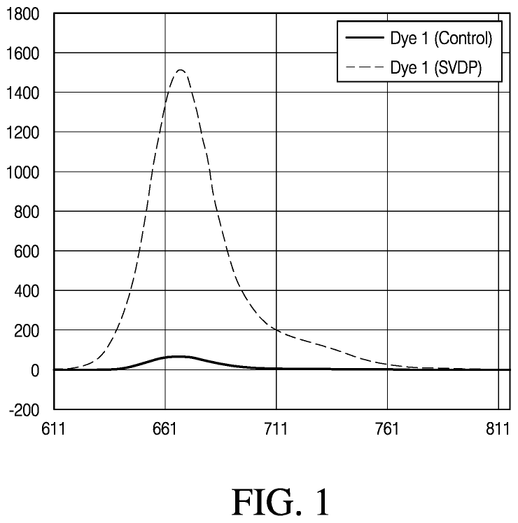

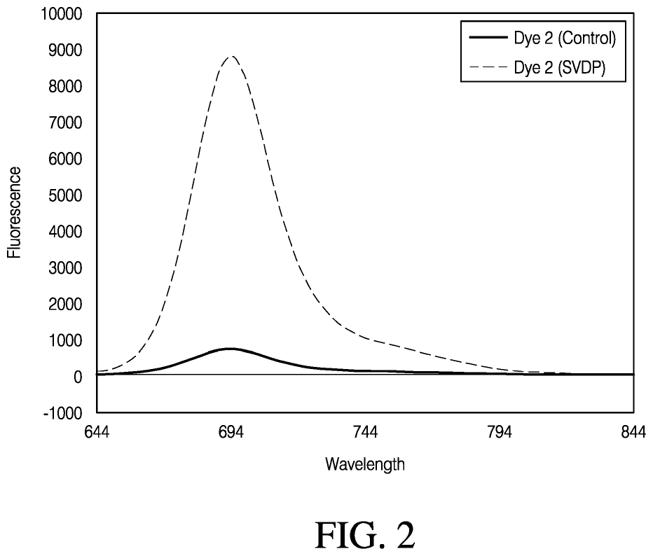

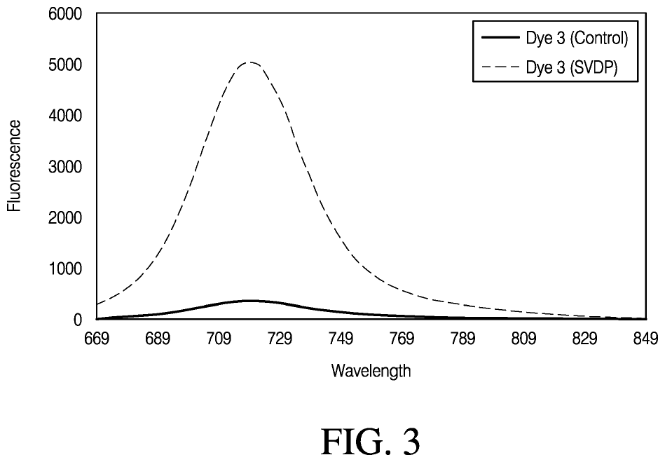

To a 2 mL microtube was added 30 μL Tris-Mg buffer (100 mM pH 7.5 Tris HCl; 20 mM MgCl₂ in nuclease-free water), 0.1 ODU (260 nm) of probe dissolved in nuclease-free water, and 2 μL snake venom from Crotalus adamanteus (2 mg/mL). The resulting solution was vortexed and placed on a heating block at 37° C. for 18 h, and then heated to 85° C. for 15 min. After cooling to room temperature, the digested sample was diluted to the appropriate concentration with 1×TE buffer, and a fluorescence emission spectrum was taken. Separately, a sample of undigested probe was made by combining 30 μL Tris-Mg buffer, 0.1 ODU of probe, and 2 μL nuclease-free water in a 2 mL microtube. This control was diluted to the same concentration with 1×TE buffer, and a fluorescence emission spectrum was taken using the same conditions as before. Comparison of the fluorescence intensities at the emission maxima was used to determine the quenching efficiency. The quenching efficacy is shown in FIG. 1 (95.5% quenching of Reporter Dye 1), FIG. 2 (91.9% quenching of Reporter Dye 2) and FIG. 3 (92.9% quenching of Reporter Dye 3).

### Stability Experiment

A base solution was formulated by mixing water (7.9 mL), tris-HCL (2M, pH 8.0, 0.500 mL), magnesium chloride (1M, 0.100 mL), bulk dATP (100 mM, 0.050 mL), bulk dCTP (100 mM, 0.050 mL), bulk dGTP (100 mM, 0.050 mL), bulk dTTP (100 mM, 0.050 mL), a nonionic detergent (tween-20) (10%, 0.020 mL), glycerol (0.400 mL), and potassium chloride (KCl) (2M, 0.250 mL).

A 200 nM probe solution was created for each probe by diluting a stock probe solution (100 μM, 1 μL) with nuclease-free water (249 μL) and the base solution (250 μL) formulated above. The stock probe solutions included the following dye and quencher combinations:
Stock Probe 1 Reporter Dye 1 with QSY™ 21 quencher,
Stock Probe 2 Reporter Dye 2 with conventional quencher,
Stock Probe 3 Reporter Dye 1 with Compound 3, and
Stock Probe 4 Reporter Dye 2 with Compound 3.

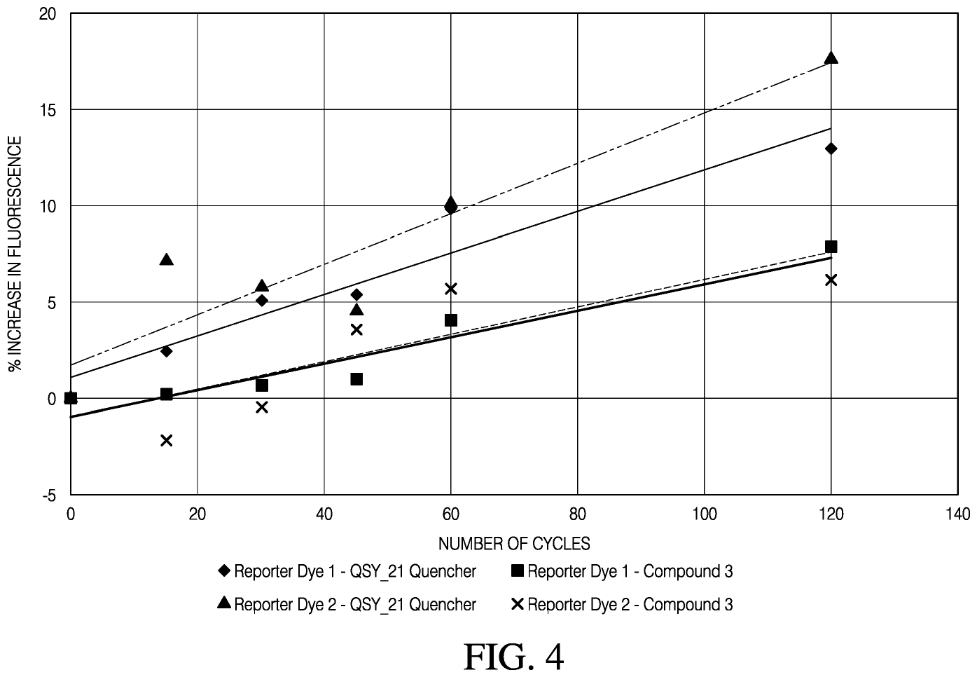

Fluorescence of each probe solution was measured in a microcuvette before and during thermocycling. Each probe solution was subjected to a thermal cycling process using a 96-well thermocycler. The thermal cycling process included the following stages:
Stage 1 (performed once) 50° C. for 2 mins,
Stage 2 (performed once) 95° C. for 2 mins,
Stage 3 (performed 60 times) 95° C. for 3 sec and 60° C. for 30 sec, and
Stage 4 (held) 5° C. The effect of the thermocycling process on the stability of the quencher compounds in Stock Probes 1-4 was evaluated. The results of this evaluation are shown in FIG. 4.

The probes including the conventional quencher with Reporter Dye 1 and 2, respectively, (the top two lines in the graph of FIG. 4) exhibited a greater increase in the percentage of fluorescence of the probe (especially over 60 thermal cycles) in comparison to the probes including Compound 3 with Reporter Dye 1 and 2, respectively, (the bottom two lines in the graph of FIG. 4). Thus, the probes including Compound 3 are more stable to thermal cycling than the probes including a conventional quencher. The inventors of the present application have surprisingly found that moving the bulky substituent groups to provide the disclosed quenchers (for instance, Compound 3) resulted in more stability than QSY™ 21 quencher.

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A compound of Formula (I): 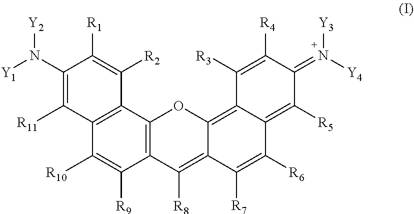
wherein
Y₁ is selected from Y₁′ and —C(O)R″,
Y₂ is selected from Y₂′ and —C(O)R″ on the condition that Y₁ and Y₂ are not both —C(O)R″;
or, alternatively, Y₁ and Y₂ form N═NR′ with the nitrogen to which they are bound;
or, alternatively, Y₁′ forms a saturated or unsaturated, substituted or unsubstituted ring with R₁/R₁₁ together with the atoms to which they are bonded, and/or Y₂′ forms a saturated or unsaturated, substituted or unsubstituted ring with R₁/R₁₁ together with the atoms to which they are bonded;
Y₃ is selected from Y₃′ and —C(O)R″,
Y₄ is selected from Y₄′ and —C(O)R″ on the condition that Y₃ and Y₄ are not both —C(O)R″;
or, alternatively, Y₃′ forms a saturated or unsaturated, substituted or unsubstituted ring with R₄/R₅ together with the atoms to which they are bonded, and/or Y₄′ forms a saturated or unsaturated, substituted or unsubstituted ring with R₄/R₅ together with the atoms to which they are bonded;
R″ is selected from —(CQ¹Q²)ₓ-R^{a};
wherein Q¹ and Q² are independently selected from hydrogen and methyl,
x is an integer ranging from 1 to 10,
R^{a} is a trimethyl quinone;
R₅, R₆, R₇, R₉, R₁₀, R₁₁ are independently selected from —H, halogens, alkyl, and alkyl group independently substituted with one or more Z₂;
R₁, R₂, R₃, R₄, Y₁′, Y₂′, Y₃′, Y₄′, and R′ are independently selected from —H, alkyl, alkyl independently substituted with one or more Z₂, heteroalkyl, heteroalkyl independently substituted with one or more Z₂, aryl, aryl independently substituted with one or more Z₂, heteroaryl, heteroaryl independently substituted with one or more Z₂, arylalkyl, arylalkyl independently substituted with one or more Z₂, heteroarylalkyl, heteroarylalkyl independently substituted with one or more Z₂, halogen, —OS(O)₂OR, —S(O)₂OR, —S(O)₂R, —S(O)₂NR, —S(O)R, —OP(O)O₂RR, —P(O)O₂RR, —C(O)OR, —NO₂, ═NRR, —NRR, —N⁺RRR, —NC(O)R, —C(O)R, —C(O)NRR, —CN, and —OR;
wherein R is independently selected from —H, alkyl, heteroalkyl, aryl, heteroaryl, arylalkyl, and heteroarylalkyl;
wherein Z₂ is selected from —R, halogen, —OS(O)₂OR, —S(O)₂OR, —S(O)₂R, —S(O)₂NR, —S(O)R, —OP(O)O₂RR—P(O)O₂RR, —C(O)OR, —NO₂, —NRR, —N⁺RRR, —NC(O)R, —C(O)R, —C(O)NRR, —CN, —O, —OR, —(CH₂)ₓ—R^{b}, —N(CH₂)ₓ—R^{b};
wherein R^{b} is selected from -halogen, —OH, —OR, —SH, —NH₂, —C(O)O, —C(O)OH, —C(O)NH₂;
R₈ is selected from —H, alkyl, alkyl independently substituted with one or more Z₁, heteroalkyl, heteroalkyl independently substituted with one or more Z₁, aryl, aryl independently substituted with one or more Z₁, heteroaryl, heteroaryl independently substituted with one or more Z₁, arylalkyl, arylalkyl independently substituted with one or more Z₁, heteroarylalkyl, and heteroarylalkyl independently substituted with one or more Z₁; and
Z₁ is selected from the group consisting of, —R*, halogen, —CR*R*R*, —OS(O)₂OR*, —S(O)₂OR*, —SO₃, —S(O)₂R*, —S(O)₂NR*, —S(O)R*, —OP(O)O₂R*R*—P(O)O₂R*R*, —C(O)OR*, —N═N—R*—R*, —NO₂—NR*R*, —N*R*R*R*, —NC(O)R*, —C(O)R*, —C(O)NR*R*, —CN, —O and —OR*, wherein R* is independently selected from —H, halogen, alkyl, heteroalkyl, —NO₂, aryl, heteroaryl, arylalkyl, heteroarylalkyl, and linking group (LG).

2. The compound of claim 1, wherein Y₁ is selected from Y₁′ and forms a saturated or unsaturated, substituted or unsubstituted ring with R₁₁ together with the atoms to which they are bonded.

3. The compound of claim 2, wherein the ring is unsaturated and substituted.

4. The compound of claim 1 or 2, wherein Y₄ is selected from Y₄′ and forms a saturated or unsaturated, substituted or unsubstituted ring with R₅ together with the atoms to which they are bonded.

5. The compound of claim 4, wherein the ring is unsaturated and substituted.

6. The compound of claim 1, wherein
Y₁ is selected from Y₁′ and forms a saturated or unsaturated, substituted or unsubstituted ring with R₁₁ together with the atoms to which they are bonded and
Y₂ is selected from Y₂′ and forms a saturated or unsaturated, substituted or unsubstituted ring with R₁/R₁₁ together with the atoms to which they are bonded.

7. The compound of claim 1, wherein
Y₁ is selected from Y₁′ and forms a saturated or unsaturated, substituted or unsubstituted ring with R₁₁ together with the atoms to which they are bonded;
Y₂ is selected from Y₂′ and forms a saturated or unsaturated, substituted or unsubstituted ring with R₁/R₁₁ together with the atoms to which they are bonded;
Y₃ is selected from Y₃′ and forms a saturated or unsaturated, substituted or unsubstituted ring with R₄ together with the atoms to which they are bonded; and
Y₄ is selected from Y₄′ and forms a saturated or unsaturated, substituted or unsubstituted ring with R₅ together with the atoms to which they are bonded.

8. The compound of claim 1, wherein one of Y₁ and Y₂ is selected from —C(O)R″.

9. The compound of claim 1, wherein one of Y₁ and Y₂ is selected from —C(O)R″ and one of Y₃ and Y₄ is selected from —C(O)R″.

10. The compound of claim 1, wherein Y₁ and Y₂ form N═NR′ with the nitrogen to which they are bound.

11. The compound of claim 10, wherein R′ is selected from aryl independently substituted with one or more Z₂.

12. The compound of claim 1, wherein R₆, R₇, R₉, and R₁₀, are each —H.

13. The compound of claim 1, wherein R₂ and R₃, are both —H.

14. The compound of claim 1, wherein R₈ is selected from 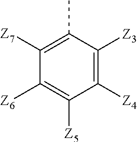
wherein Z₃, Z₄, Z₅ Z₆, and Z₇ each taken separately are independently selected from Z₁.

15. The compound of claim 14, wherein Z₃, Z₄, Z₅, Z₆, and Z₇ are each independently selected from —H, halogen, lower alkyl, —CR*R*R*, —C(O)OR*, —C(O)R*, —S(O)OR*, S(O)₂R*, —SO₃, —N═N—R*—R*, and —CH₂OR*.

16. The compound of claim 14, wherein at least one of Z₃, Z₄, Z₅, Z₆, and Z₇ is —F or —Cl.

17. The compound of claim 15, wherein at least one of Z₃, Z₄, Z₅, Z₆, and Z₇ is —CR*R*R* and R* is —F or —Cl.

18. The compound of claim 14, wherein Z₃ is —C(O)OH.

19. The compound of claim 14, wherein one of Z₅ or Z₆ is —C(O)OH.

20. The compound of claim 14, wherein Z₃ is —S(O)OH and one of Z₅ or Z₆ is —C(O)OH.

21. The compound of claim 14, wherein Z₃ is —C(O)OR* and one of Z₄, Z₅, Z₆, or Z₇ is linking group.

22. The compound of claim 1, wherein R₈ is selected from 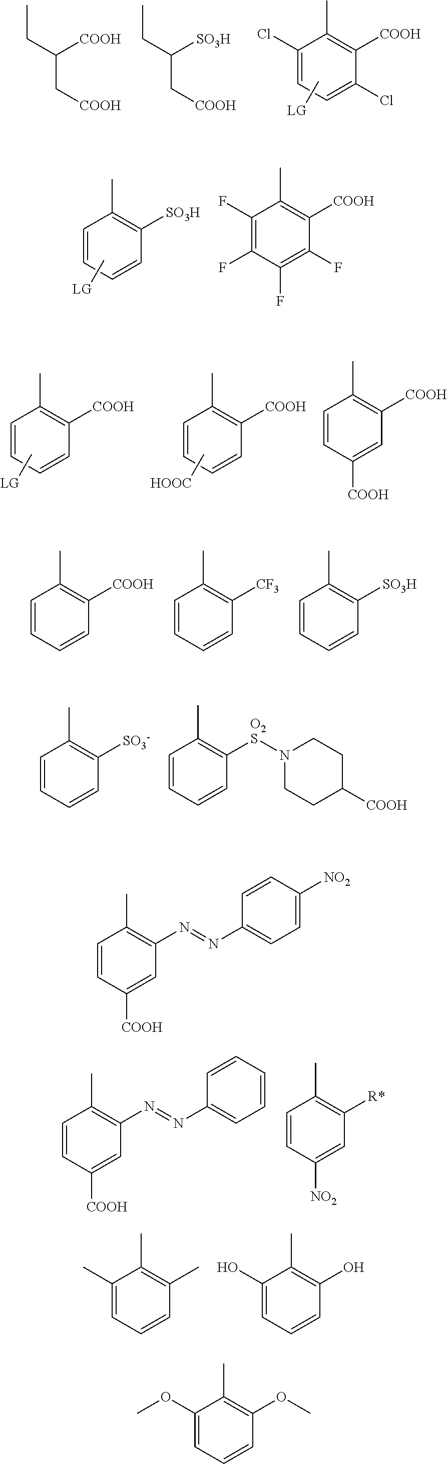
wherein LG is linking group.

23. A compound selected from Table Q and protected forms thereof.

24. A compound selected from any one of claims 1-23 attached to a solid support.

25. An oligonucleotide probe, comprising:
a) a fluorophore; and
b) a quenching compound according to any one of claims 1-23; and
c) an oligonucleotide, wherein the fluorophore and the quenching compounds are covalently attached to the oligonucleotide.

26. The oligonucleotide probe of claim 25 attached to a solid support.

27. An oligonucleotide probe composition comprising an oligonucleotide probe of claim 25 and an aqueous medium.

28. The oligonucleotide probe composition of claim 27, further comprising a polymerase.

29. The oligonucleotide probe composition of claim 28, wherein the polymerase is a DNA polymerase.

30. The oligonucleotide probe composition of claim 28, wherein the polymerase is thermostable.

31. The oligonucleotide probe composition of claim 27, wherein the composition further comprises a reverse transcriptase (RT).

32. The oligonucleotide probe composition of claim 27, further comprising at least one deoxyribonucleoside triphosphate (dNTP).

33. A composition comprising:
a) a quenching compound of any one of claims 1-24; and
b) a nucleic acid molecule.

34. The composition of claim 33, further comprising an enzyme.

35. A method of detecting or quantifying a target nucleic acid molecule in a sample by polymerase chain reaction (PCR), the method comprising:
(i) contacting the sample comprising one or more target nucleic acid molecules with a) at least one oligonucleotide probe having a sequence that is at least partially complementary to the target nucleic acid molecule, where the at least one probe undergoes a detectable change in fluorescence upon amplification of the one or more target nucleic acid molecules; and with b) at least one oligonucleotide primer pair;
(ii) incubating the mixture of step (i) with a DNA polymerase under conditions sufficient to amplify one or more target nucleic acid molecules; and
(iii) detecting the presence or absence or quantifying the amount of the amplified target nucleic acid molecules by measuring fluorescence of the oligonucleotide probe, wherein the oligonucleotide probe comprises:
a) a fluorophore;
b) a quenching compound of any one of claims 1-24; and
c) an oligonucleotide linker joining the dye and the quenching compound.

36. The method of claim 35, wherein the PCR is real-time or quantitative PCR (qPCR).

37. The method of claim 35, wherein the polymerase is a Taq polymerase.

38. A conjugate, comprising:
a) a fluorescent donor compound, wherein the fluorescent donor compound emits light at a wavelength in the visible or near-infrared region of the electromagnetic spectrum upon excitation at an appropriate wavelength and having an initial fluorescence intensity,
b) a quenching acceptor compound, wherein the quenching acceptor compound is a substituted 3-imino-3H-dibenzo[c,h]xanthen-11-amine, and
c) a linking compound, wherein the fluorescent donor compound and the quenching acceptor compound are attached to the linking compound, wherein the distance between the donor compound and acceptor compound is such that upon excitation at the appropriate wavelength the initial fluorescence intensity of the fluorescent donor compound is reduced by a detectable amount.

39. The conjugate of claim 38, wherein the quenching acceptor compound is a compound of claim 1.
